(19) United States
(12) Patent Application Publication
(10) Pub. No.: US 2024/0132534 A1
(43) Pub. Date: Apr. 25, 2024

(54) OLIGONUCLEOTIDE PRIMER WITH AN ACYCLIC NUCLEOSIDE STRUCTURE FOR INITIAL CAPPING

(71) Applicant: JIANGSU SYNTHGENE BIOTECHNOLOGY CO., LTD., Nanjing (CN)

(72) Inventors: Jiaying MIAO, Nanjing (CN); Lei HUANG, Nanjing (CN); Qi SHEN, Nanjing (CN)

(73) Assignee: JIANGSU SYNTHGENE BIOTECHNOLOGY CO., LTD., Nanjing (CN)

(21) Appl. No.: 18/520,609

(22) Filed: Nov. 28, 2023

Related U.S. Application Data
(63) Continuation-in-part of application No. PCT/CN2023/091598, filed on Apr. 28, 2023.

(30) Foreign Application Priority Data
May 5, 2022 (CN) 202210480431.7

(51) Int. Cl. C07H 21/02; C12Q 1/6853

(52) U.S. Cl. CPC C07H 21/02; C12Q 1/6853

(57) ABSTRACT

Provided is an oligonucleotide primer with an acyclic nucleoside structure for initial capping, which has a molecular structural formula of ^{m7}UNGpppA_{2′ome}pG, and has higher mRNA in vitro transcription efficiency, higher capping efficiency, lower immunogenicity and higher protein translation efficiency.

This application is a continuation-in-part (CIP) application based upon International Patent Application No. PCT/CN2023/091598 filed Apr. 28, 2023, which claims the priority of Chinese Patent Application No. 202210480431.7, filed with the China National Intellectual Property Administration on May 5, 2022, and titled with “OLIGONUCLEOTIDE PRIMER WITH AN ACYCLIC NUCLEOSIDE STRUCTURE FOR INITIAL CAPPING”, the disclosures of each of which are hereby incorporated by reference in their entirety.

### FIELD

The present disclosure relates to the technical fields of chemistry and bioengineering, and in particular to an oligonucleotide primer with an acyclic nucleoside structure for initial capping.

### BACKGROUND

In eukaryotic cells, most messenger RNAs (mRNAs) are blocked, or “capped” to the 5′ ends, where the cap contains a 5′-5′ triphosphate linkage between the two nucleoside moieties and a 7-methyl group on the distal guanine ring. Such capping promotes normal mRNA function in the cell. In vitro transcription for synthesizing mRNA has become an important tool for introducing exogenous genes and expressing proteins, and is widely used in the treatment and prevention of diseases. In vitro transcription for synthesizing mRNA allows preparation of RNA molecules that perform appropriately in various biological applications, including research applications and commercial production of polypeptides, for example, the production of polypeptides containing “unnatural” amino acids at specific sites in cell-free translation systems, or the production in cultured cells of polypeptides that require post-translational modification for their activity or stability. In the latter system, synthesis takes a significantly longer time and therefore more protein is produced. The in vitro transcription yield of mRNA, the capping rate of the mRNA in the product, and the 5′-position capping analog are key processes in the preparation of mRNA.

The cap analogs have evolved from the first generation of mCap to the second generation of anti-reverse cap analogs (ARCA) and the third generation of Cap 1 analogs (CleanCap).

The first-generation cap analog mCap is a standard cap structural analog. m7Gppp G is likely to incorporate into m7G (5′) ppmG-RNA or G (5′) pppm7G-RNA in two directions due to the presence of two free 3′-OH moieties, producing transcripts that are a mixture of 5′-capped and 5′-triphosphate transcripts. The reverse cap structure formed by the incorporation of the cap analog in the wrong direction binds poorly to eIF4E, leading to inefficient translation of mRNA and ultimately causing a low yield of the target protein.

The second-generation cap analog is anti-reverse cap analog (ARCA). ARCA has been modified with a methoxy group at the third position and has only one 3′-OH group, resulting in directional integration during co-transcription. Therefore, it can only be inserted in the correct direction during the transcription process, and the resulting mRNA is translated twice as efficiently as the mRNA formed by mCap. In the transcription reaction, a mixture of ARCA and GTP in a ratio of 4:1 will yield approximately 70% capped mRNA. ARCA is co-transcribed to generate mRNA with Cap 0 structure. 1 μg of the starting template produces about 30 pg of mRNA product through transcription. However, the transcription yield is low, and further action of dioxymethyltransferase is required to generate mRNA with Cap1 structure.

The third-generation Cap1 analog (CleanCap) can be co-transcribed to directly generate mRNA with Cap1 structure and the capping efficiency is increased to approximately 90%.

Patent CN201680067458.6 reports a composition and method for synthesizing 5′-capped RNA, wherein the oligonucleotide primer for initial capping has the general form of m⁷Gppp[N_{2, Ome}]ₙ[N]ₘ, where m⁷G is N₇-methylated guanosine or any guanosine analog, N is any natural, modified or non-natural nucleoside, “n” can be any integer from 0 to 4 and “m” can be an integer from 1 to 9. Cleancap belongs to Cap1. Unlike ARCA, which uses a dimer (m⁷GpppG) to initiate T7 transcription, CleanCap uses a trimer (m⁷GpppAmG) to initiate T7 transcription. The method has a relatively high yield that 4 mg of capped RNA is prepared per milliliter of transcription reaction system, and a capping efficiency of 90%. The immunogenicity of its transcription products is also lower than that of ARCA.

Patent U.S. Pat. No. 10,968,248B2 discloses Trinucleotide mRNA cap analogs, involving trinucleotide cap analogs for improving in vitro synthesis of mRNA and transcription of m⁷G(5′)p₃-RNA. In this structure, the third nucleotide in the cap structure is replaced by acyclic UNA, which is not conducive to the recognition of T7 RNA polymerase since it is the starting nucleotide of transcription. Therefore, the capping efficiency and the in vitro transcription efficiency are both reduced.

Therefore, there is an urgent need in this field to develop a capping analog combination, so that the mRNA synthesized through in vitro transcription can achieve higher in vitro transcription yield, higher capping efficiency, and lower immunogenicity.

### SUMMARY

In order to solve the problems of insufficient in vitro transcription yield and capping efficiency in the prior art, the present application provides an oligonucleotide with an acyclic nucleoside structure as a primer for initial capping and transcription. Through the co-transcriptional capping method, the oligonucleotide primer with an acyclic nucleoside structure for initial capping can be directly used for in vitro transcription to generate mRNA with capped structure. The oligonucleotide primer with an acyclic nucleoside structure for initial capping contains a UNA (unlocked nucleic acid) structure to replace the original five-membered sugar ring structure, and has a good anti-reverse transcription effect during in vitro transcription of mRNA since UNA fails to serve as the starting site for transcription, which ultimately leads to higher capping efficiency of mRNA. The acyclic structure of UNA helps mRNA escape recognition by the immune system in the body and better reduces immunogenicity. In addition, due to the introduction of the non-natural nucleotide UNA, the mRNA is not easily hydrolyzed by ribozymes, which increases the stability of the mRNA in vivo after capping.

The present disclosure provides an oligonucleotide primer with an acyclic nucleoside structure for initial capping, having a structure of

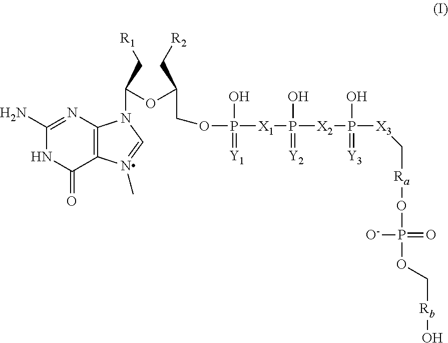

wherein, R₁ and R₂ are independently selected from the group consisting of H, OH, alkyl, O-alkyl, and halogen;

X₁, X₂ and X₃ are independently selected from the group consisting of O, CH₂ and NH;

Y₁, Y₂ and Y₃ are independently selected from the group consisting of O, S, Se and BH₃;

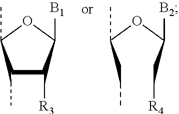

Rₐ and R_{b} are independently

R₃ and R₄ are independently selected from the group consisting of hydrogen, hydroxyl, halogen, substituted or unsubstituted O-alkyl, substituted or unsubstituted S-alkyl, substituted or unsubstituted NH-alkyl, substituted or unsubstituted N-dialkyl, substituted or unsubstituted O-aryl, substituted or unsubstituted S-aryl, substituted or unsubstituted NH-aryl, substituted or unsubstituted O-aralkyl, substituted or unsubstituted S-aralkyl, and substituted or unsubstituted NH-aralkyl; and

B₁ and B₂ are independently natural, modified, or non-natural nucleobases.

In some embodiments, in the oligonucleotide primer with an acyclic nucleoside structure for initial capping, X₁, X₂ and X₃ are independently O; and

Y₁, Y₂ and Y₃ are independently O.

In some embodiments, in the oligonucleotide primer with an acyclic nucleoside structure for initial capping, R_{b} is

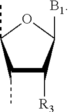

In some embodiments, in the oligonucleotide primer with an acyclic nucleoside structure for initial capping, Rₐ and R_{b} are independently

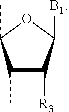

In some embodiments, in the oligonucleotide primer with an acyclic nucleoside structure for initial capping, R₁ and R₂ are independently selected from the group consisting of H, OH, halogen, alkyl, and substituted or unsubstituted O-alkyl.

In some embodiments, in the oligonucleotide primer with an acyclic nucleoside structure for initial capping, R₃ and R₄ are independently selected from the group consisting of hydrogen, hydroxyl, halogen, and substituted or unsubstituted O-alkyl.

In some embodiments, in the oligonucleotide primer with an acyclic nucleoside structure for initial capping, R₁ and R₂ are independently selected from the group consisting of H, OH, F, methyl, ethyl, methoxy, ethoxy, —O-methylene-O-methyl, —O-ethylene-O-methyl, —O-methylene-O-ethyl, and —O-ethylene-O -ethyl.

In some embodiments, in the oligonucleotide primer with an acyclic nucleoside structure for initial capping, R₃ and R₄ are independently selected from the group consisting of H, OH, F, methoxy, ethoxy, —O-methylene-O-methyl, —O-ethylene-O-methyl, —O-methylene-O-ethyl, and —O-ethylene-O-ethyl.

In some embodiments, the oligonucleotide primer with an acyclic nucleoside structure for initial capping has a structure selected from the group consisting of:

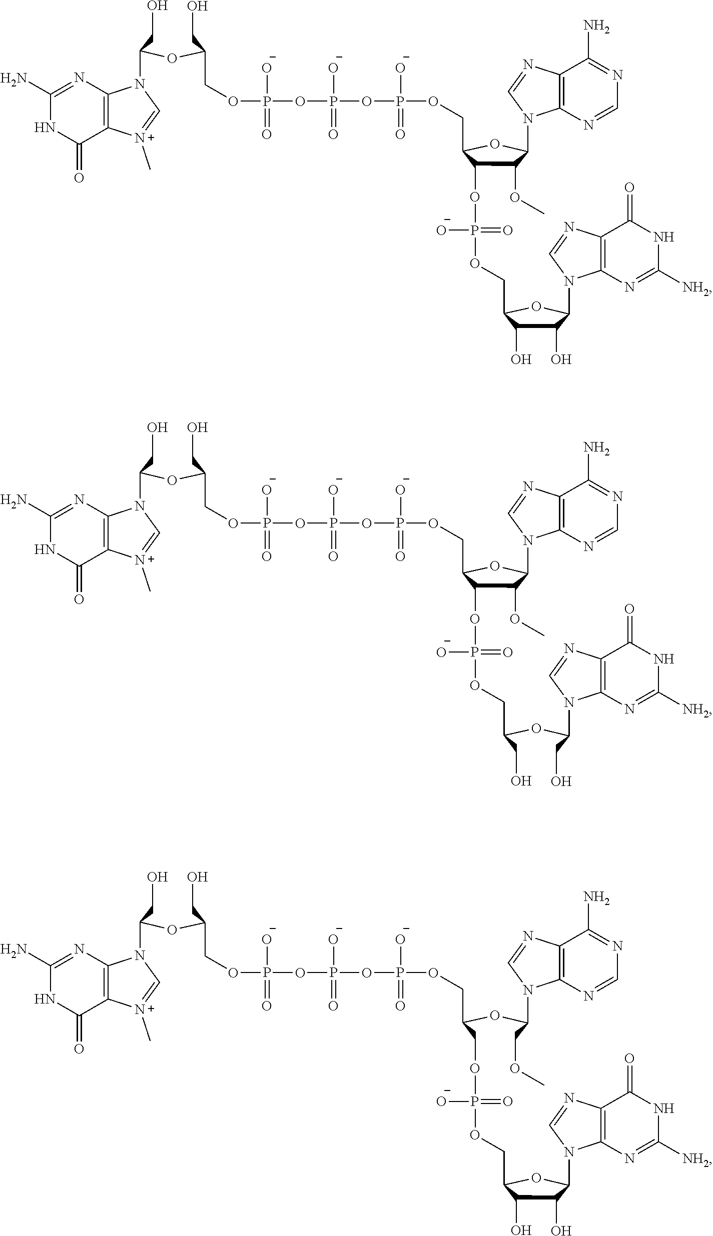

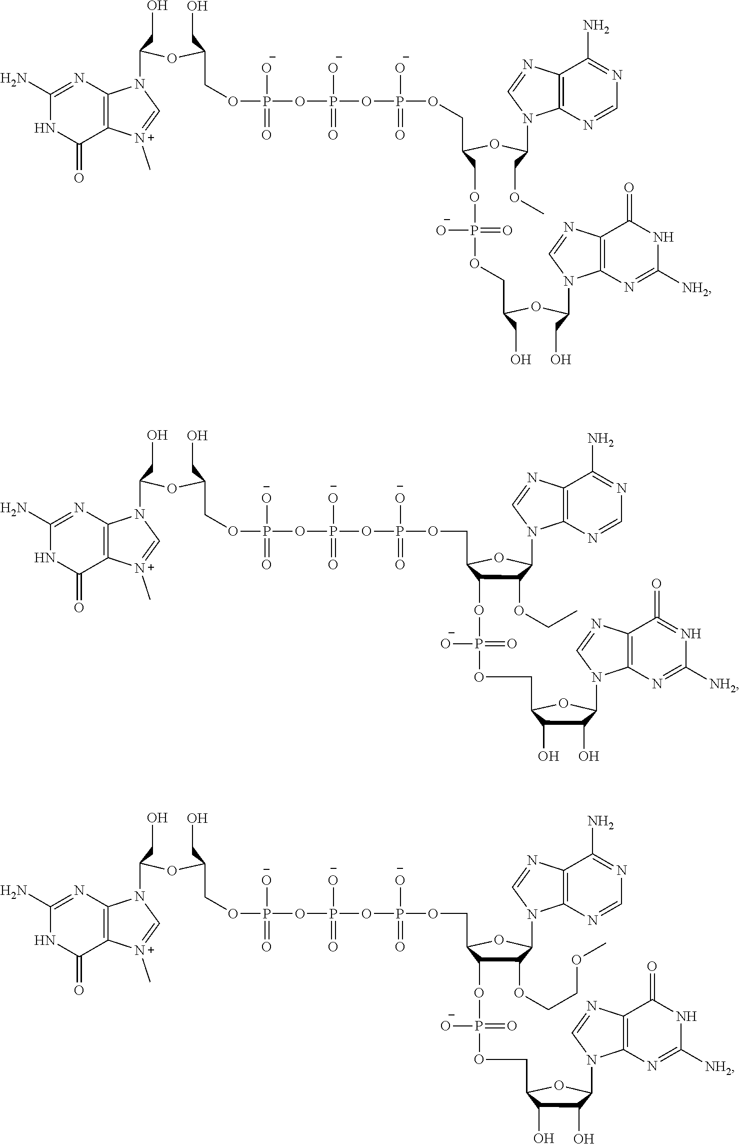

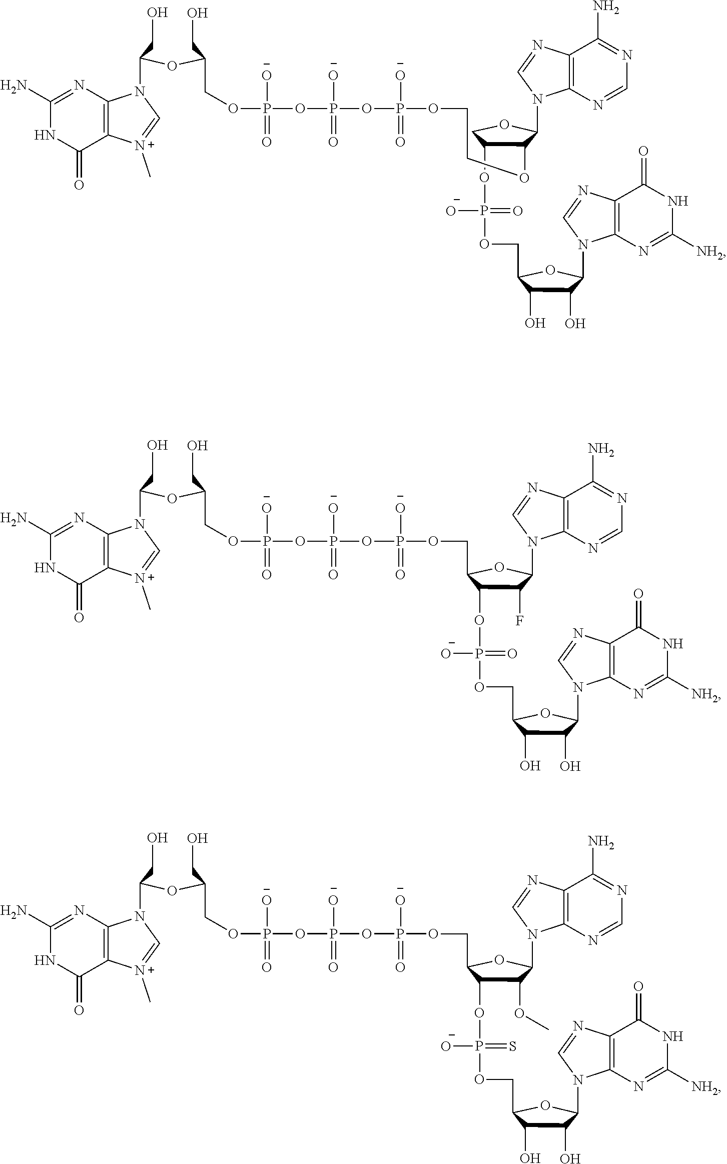

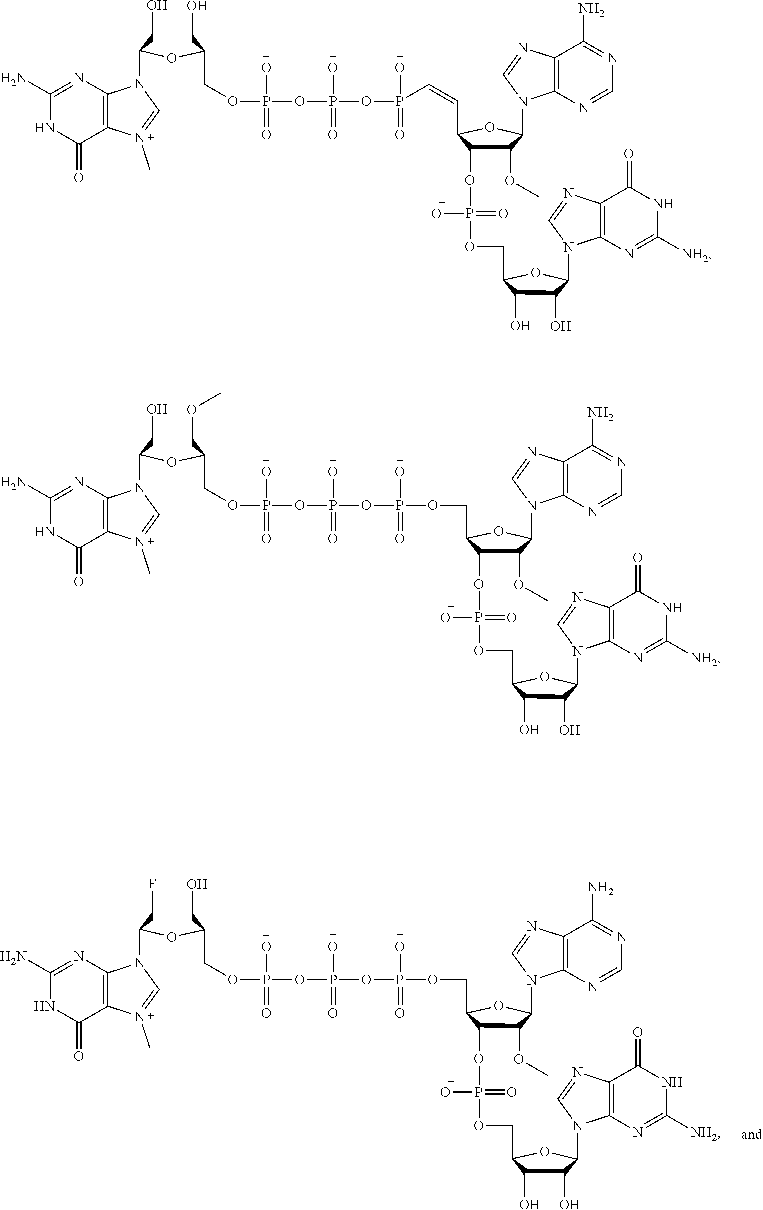

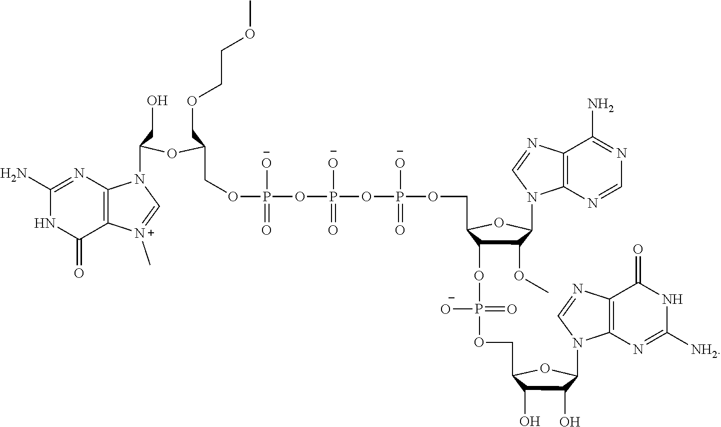

In some embodiments, the oligonucleotide primer with an acyclic nucleoside structure for initial capping has a structure selected from the group consisting of:

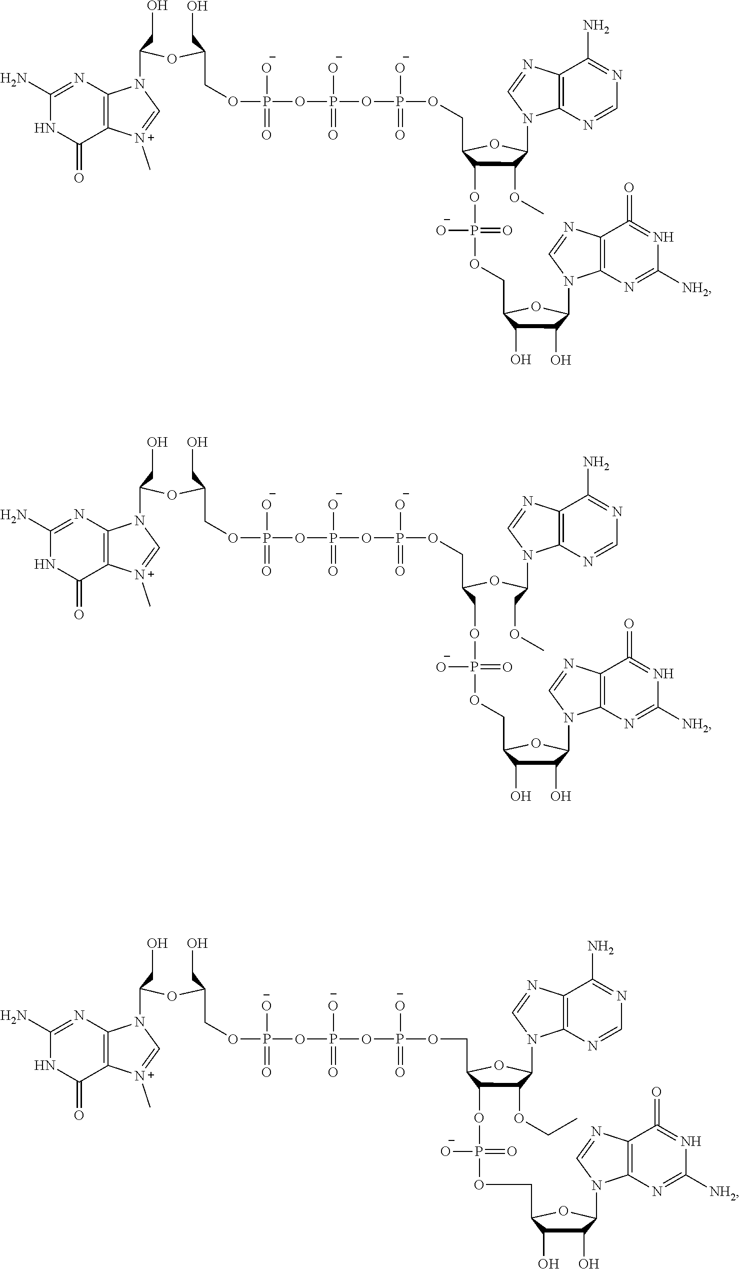

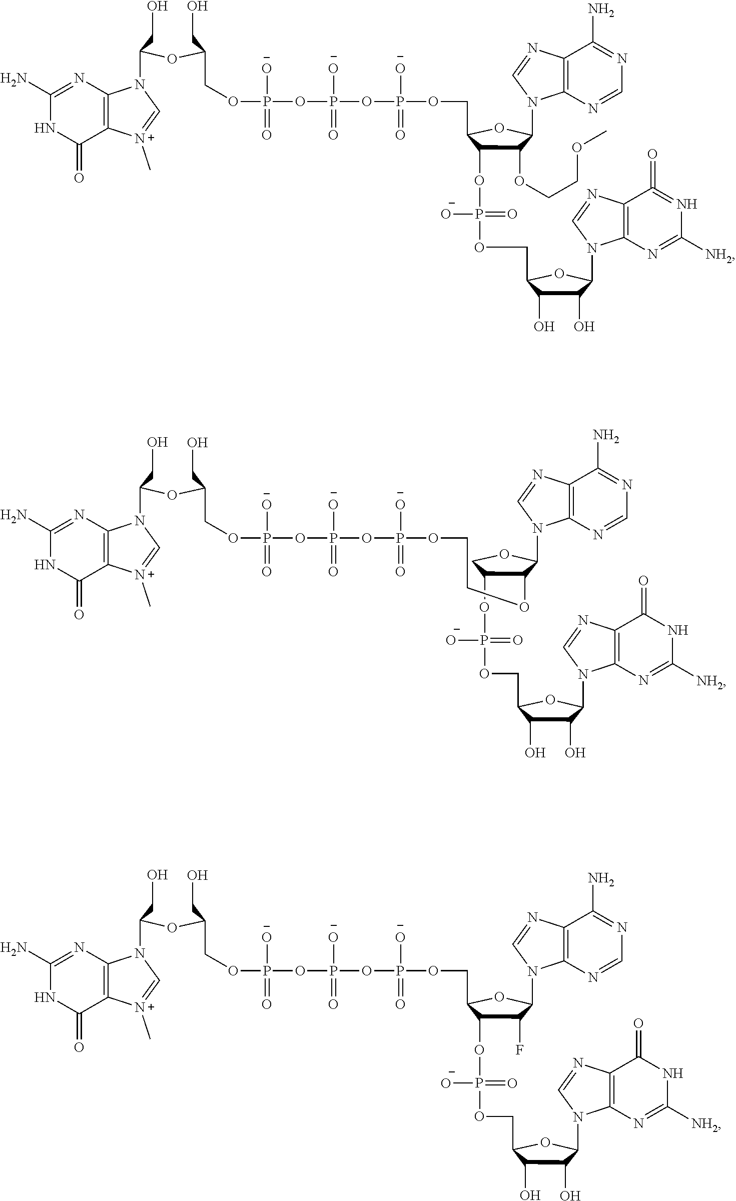

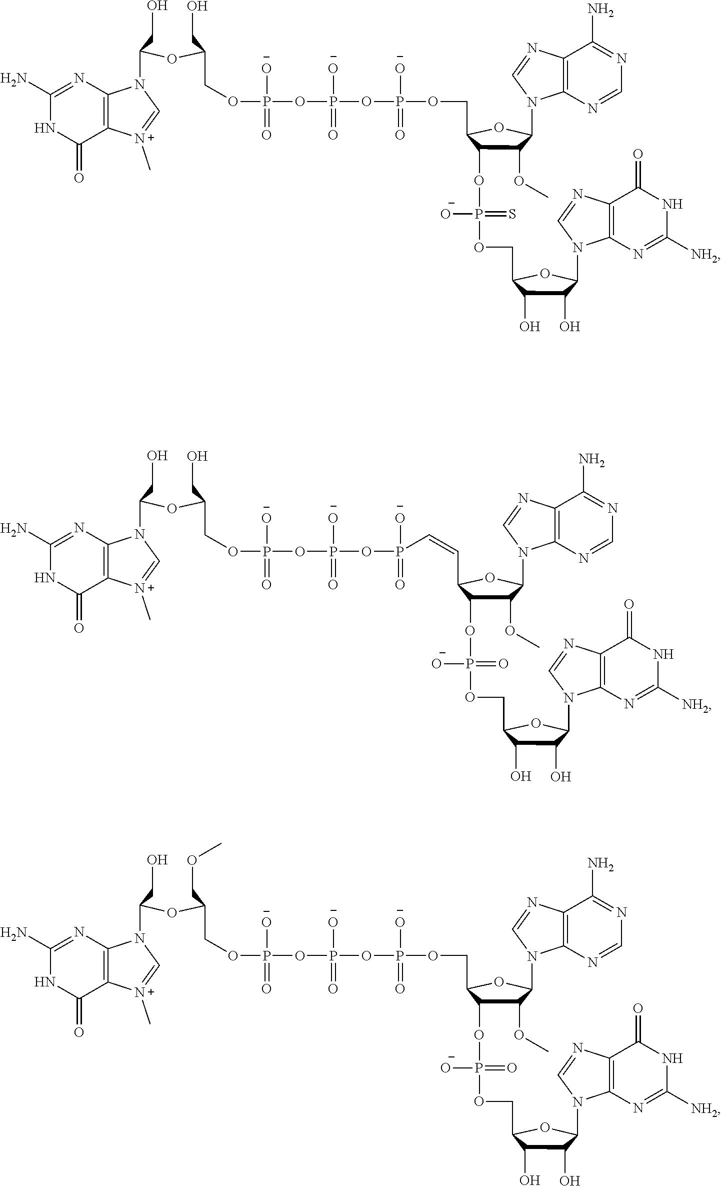

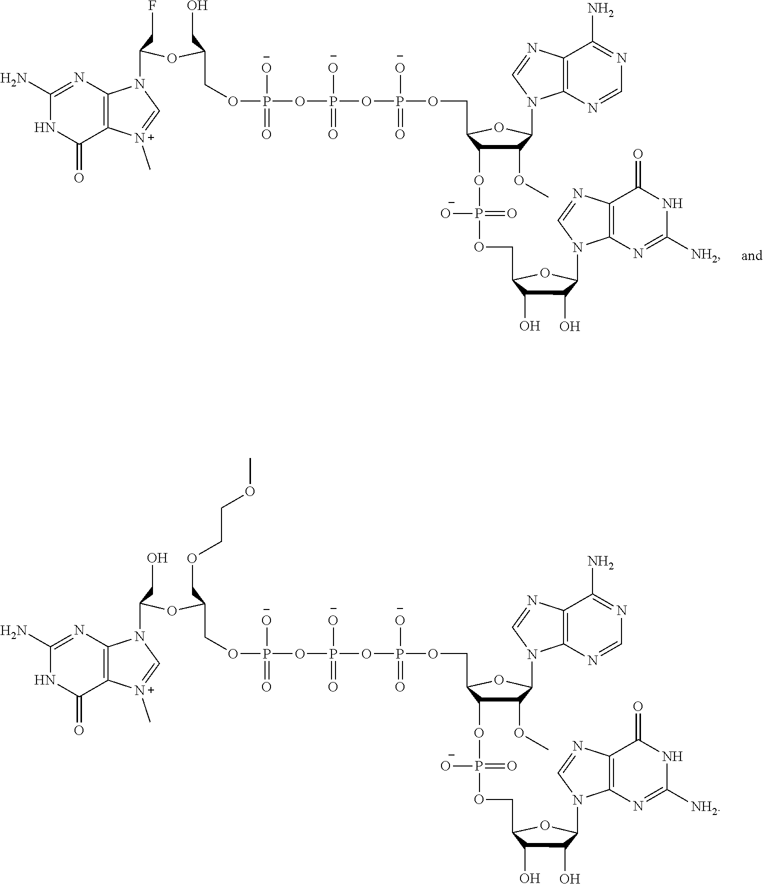

In some embodiments, the oligonucleotide primer with an acyclic nucleoside structure for initial capping has a structure selected from the group consisting of:

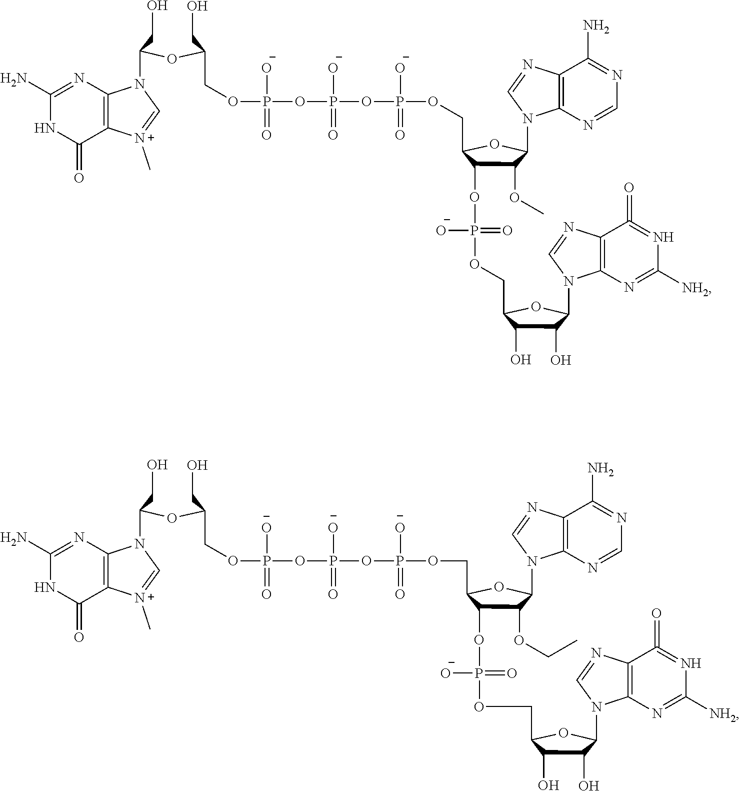

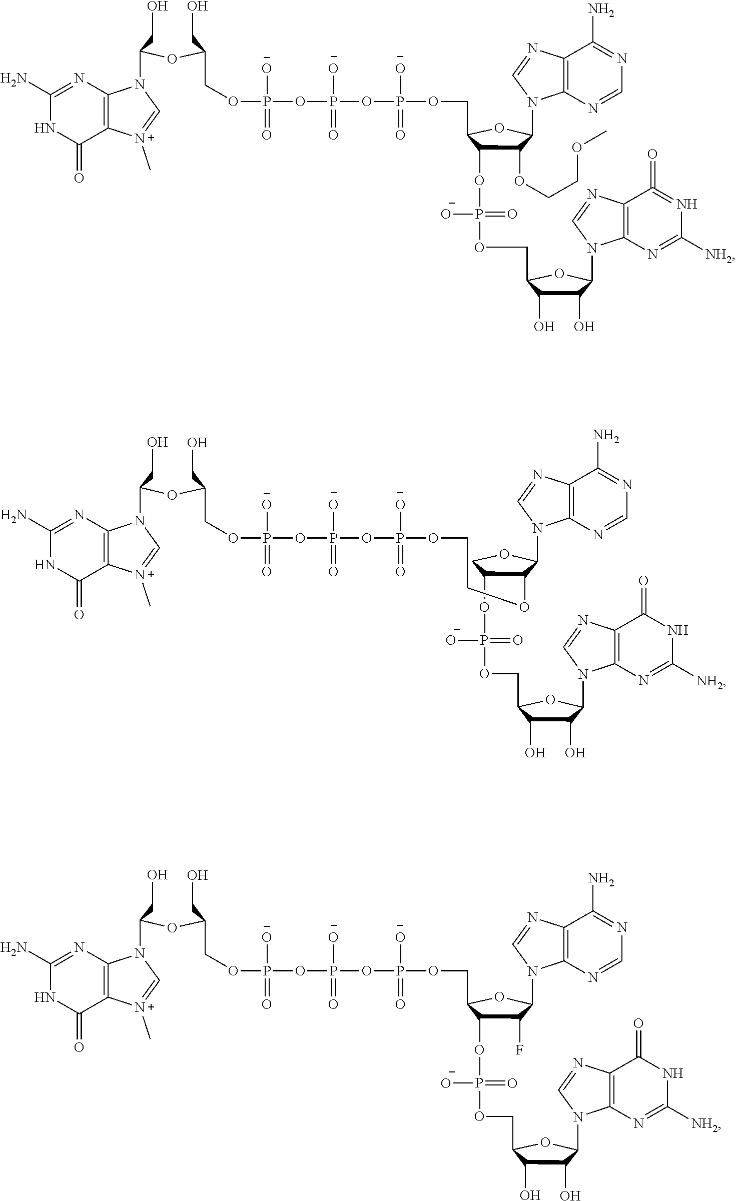

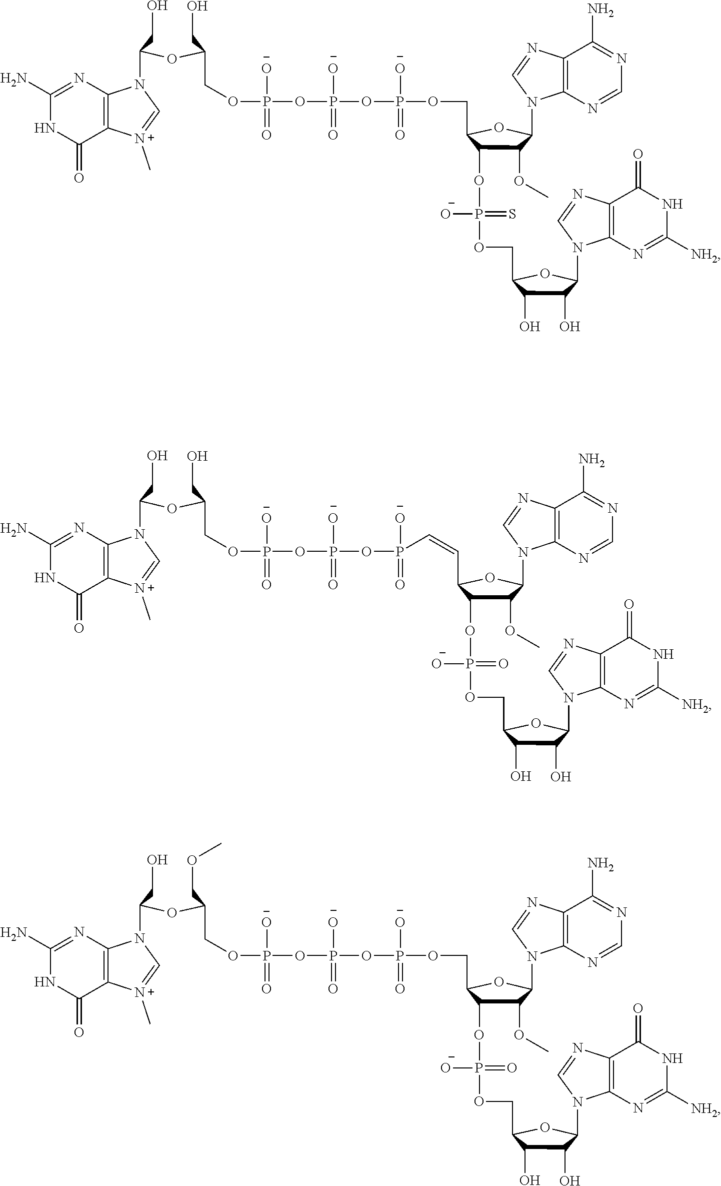

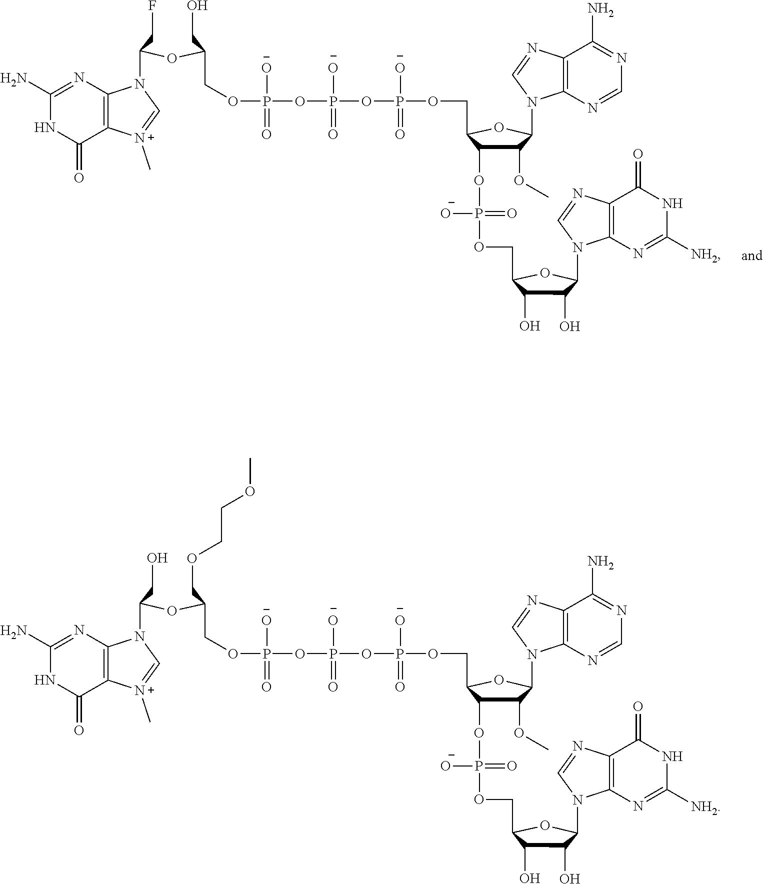

In some embodiments, the oligonucleotide primer with an acyclic nucleoside structure for initial capping has a structure selected from the group consisting of:

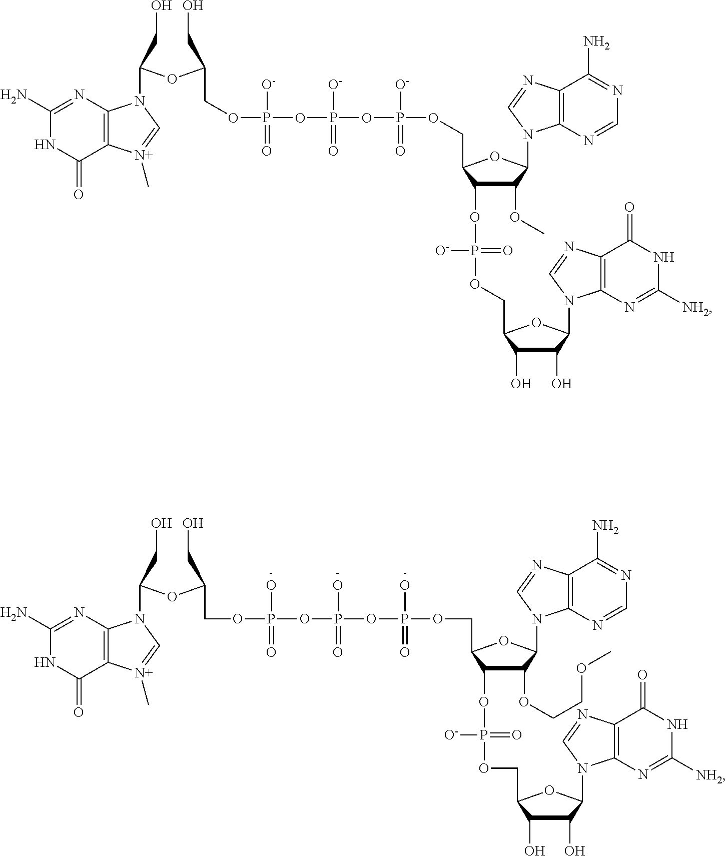

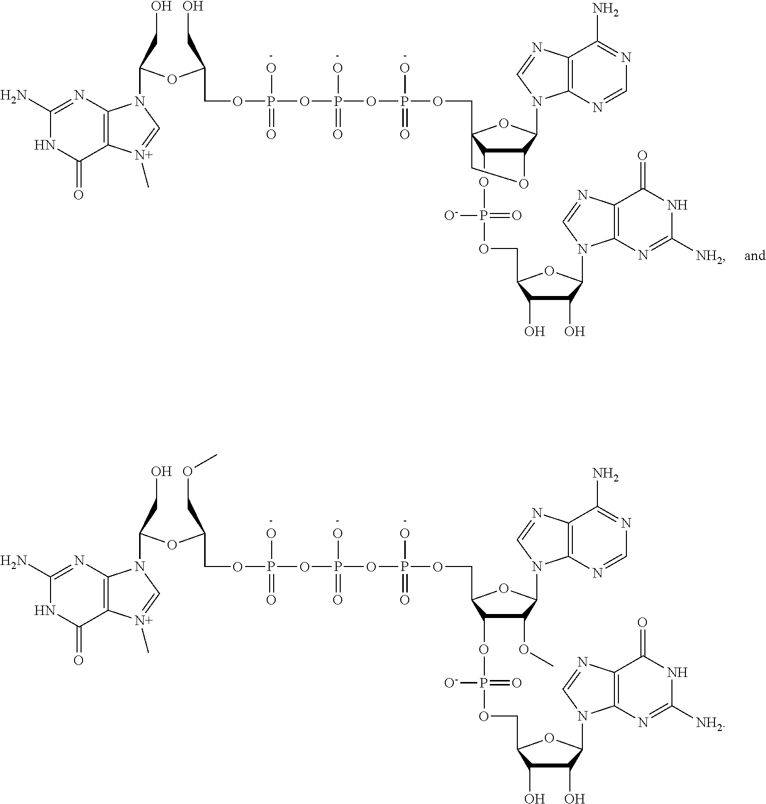

In some embodiments, the oligonucleotide primer with an acyclic nucleoside structure for initial capping has a structure of:

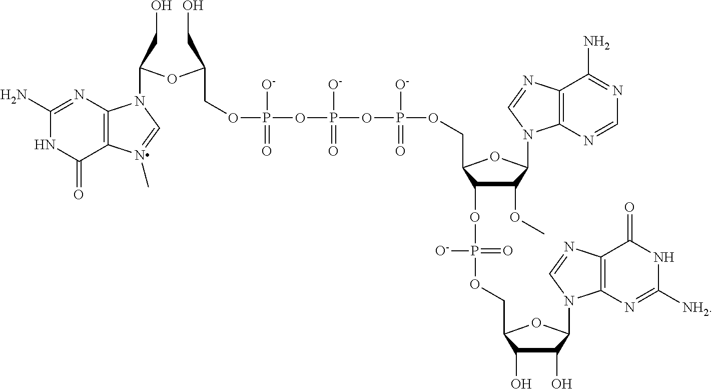

The present disclosure provides a method for preparing the oligonucleotide primer with an acyclic nucleoside structure for initial capping, comprising steps of: (1) synthesizing m7UrGDP-Im: synthesizing sugar ring-opening acyclic nucleoside from guanosine, and subjecting the acyclic nucleoside to diphosphorylation, N7 methylation, and polyphosphate imidazolation sequentially to synthesize m7UrGDP-Im; (2) preparing a phosphate bond-linked dinucleotide: coupling acyclic or non-acyclic phosphoramidite monomer with acyclic or non-acyclic disubstituted nucleoside monomer under the action of tetrazole to form a first phosphate bond, removing a protecting group by acid, then introducing a second phosphate, and finally performing hydrolyzation to obtain a phosphate bond-linked dinucleotide; and (3) synthesizing an oligonucleotide primer with an acyclic nucleoside structure for initial capping: reacting m7UrGDP-Im with the phosphate bond-linked dinucleotide to obtain the oligonucleotide primer with an acyclic nucleoside structure for initial capping;

wherein, the phosphoramidite monomer has a structure of

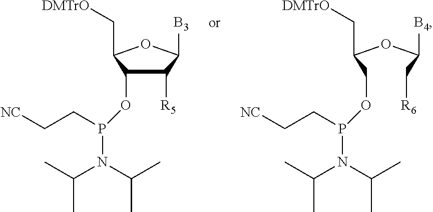

wherein, R₅ and R₆ are independently selected from the group consisting of H, OH, alkyl, O-alkyl, and halogen; and B₃ and B₄ are independently natural, modified, or non-natural nucleobases.

The disubstituted nucleoside monomer is selected from the group consisting of

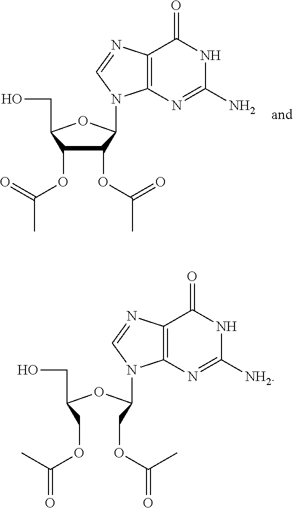

The method for preparing the oligonucleotide primer with an acyclic nucleoside structure for initial capping specifically comprises the following steps:

(1) synthesis of m7UrGDP-Im:

(1-1) weighing guanosine, dispersing it in DMF, controlling the internal temperature of the reaction solution at 5-10° C. by an ice bath, and adding TB SC1 in two batches; precipitating the product with water after the reaction being completed, performing filtration and washing the filter cake to obtain target compound B;

(1-2) weighing compound B, dispersing it in acetonitrile, adding sodium periodate, and heating the reaction to 50±5° C.; adding water after the reaction being completed, and performing filtration to obtain target compound C;

(1-3) weighing compound C, dissolving it in anhydrous methanol, cooling the reaction solution to 0±5° C., adding sodium borohydride, stirring thoroughly, and monitoring the reaction by HPLC; slowly adding ice water to quench the reaction after the reaction being completed, and then concentrating to dryness to obtain compound D; then dissolving compound D in water, adjusting the pH to 3 with 2 M hydrochloric acid, and performing purification by reverse chromatography to obtain target compound E;

(1-4) dissolving compound E in trimethyl phosphate, cooling the reaction solution to 0±5° C., and slowly adding phosphorus oxychloride dropwise; after reacting at low temperature for 4-5 h, adding 2M ammonium acetate solution to quench the reaction, and performing purification by reverse-phase chromatography to obtain target compound F; fully reacting the obtained compound F with triphenylphosphine, dithiodipyridine, and imidazole, adding a 4M sodium perchlorate acetone solution to the reaction solution for precipitation, and washing the filter cake fully with acetone to obtain target compound G;

(1-5) weighing target compound G, dissolving it in DMF, adding tributylamine phosphate and stirring thoroughly to obtain target compound H; adding aqueous solution to the reaction solution, cooling the reaction solution to 0±5° C. and slowly adding dimethyl sulfate dropwise, during which adjusting the pH to no more than 5 with 2M sodium hydroxide; monitoring the reaction by HPLC, and after the reaction being completed, performing purification by ion chromatography to obtain target compound I;

(1-6) dissolving compound I in DMF, fully reacting it with triphenylphosphine,

dithiodipyridine, and imidazole, adding a 4M sodium perchlorate acetone solution to the reaction solution for precipitation, and washing the filter cake fully with acetone to obtain target compound m7UrGDP-Im;

(2) preparation of phosphate bond-linked dinucleotides:

weighing the acyclic or non-acyclic phosphoramidite monomer into a one-neck bottle, dissolving it in dichloromethane, then adding the acyclic or non-acyclic disubstituted nucleoside monomer, cooling to 25±2° C., adding tetrazole under nitrogen blowing, heating to 25±2° C. for reaction; after the reaction being monitored to be completed, adding the iodopyridine solution to the reaction solution; after the reaction being monitored to be completed, spining to dryness, dissolving the concentrated ointment in dichloromethane, and adding trifluoroacetic acid; after the reaction being monitored to be completed, spining to dryness, slurrying with petroleum ether/dichloromethane in a certain ratio, and performing filtration to obtain intermediate A2; dissolving A2 in acetonitrile, adding phosphine reagent and tetrazole, and stirring thoroughly for reaction; after the reaction being monitored to be completed, adding the iodopyridine solution to the reaction solution; after the reaction being monitored to be completed, spining to dryness, adding methanol and concentrated ammonia in the spin bottle, reacting at room temperature for 4 h, and monitoring the reaction; after the reaction being completed, spining to dryness, adding ultrapure water, introducing to reverse ion permeation equipment, washing, concentrating, and freeze-drying to obtain the target compound phosphate bond-linked dinucleotides;

(3) synthesis of an oligonucleotide primer with an acyclic nucleoside structure for initial capping:

dissolving the m7UrGDP-Im obtained in step (1) in the D1VIF solution containing MnCl₂, adding the mixture to the DMF solution of the phosphate bond-linked dinucleotide obtained in step (2), stirring the reaction at room temperature, and terminating the reaction with 0.25M EDTA solution after 24 h; loading the mixture onto a DEAE Sephadex column (30×500 cm), eluting the product using a linear gradient of 0-1.0 M TEAB eluent, and collecting the eluted product with HPLC purity >97%; concentrating the above separated liquid, loading it into a strong anionic resin, eluting the product using a linear gradient of 0-1.0 M sodium acetate eluent, and collecting the eluted product with HPLC purity >98.5%; combining the high-purity eluates, removing the residual sodium acetate solution through nanofiltration equipment, and concentrating to obtain the target product: the oligonucleotide primer with an acyclic nucleoside structure for initial capping.

The present disclosure provides a method for in vitro co-transcriptional capping for RNA, comprising steps of: (1) providing the oligonucleotide primer with an acyclic nucleoside structure for initial capping;

(2) providing a DNA template; and

(3) performing in vitro transcription of mRNA.

The present disclosure provides a kit comprising the oligonucleotide primer with an acyclic nucleoside structure for initial capping.

The present disclosure provides a complex comprising the oligonucleotide primer with an acyclic nucleoside structure for initial capping and a DNA template, wherein the DNA template comprises a promoter region containing a transcription start site, the transcription start site has a first nucleotide at nucleotide position +1 and a second nucleotide at nucleotide position +2, the base in Rₐ of the oligonucleotide primer with an acyclic nucleoside structure for initial capping is complementary to the nucleobase at position +1 of the DNA template, and the base in R_{b} of the oligonucleotide primer with an acyclic nucleoside structure for initial capping is complementary to the nucleobase at position +2 of the DNA template.

The present disclosure provides an mRNA molecule comprising the oligonucleotide primer with an acyclic nucleoside structure for initial capping.

The present disclosure provides an oligonucleotide primer with an acyclic nucleoside structure for initial capping. The oligonucleotide primer with an acyclic nucleoside structure for initial capping provided by the present disclosure is suitable for mRNA produced by in vitro co-transcription methods with DNA sequences as templates. The DNA sequences can be derived from or modified from viruses, animals, plants and other species. Furthermore, the mRNA produced by it has higher in vitro transcription efficiency, higher capping efficiency, lower immunogenicity and higher protein translation efficiency.

Compared with the prior art, the present disclosure has the following advantages:

Compared with the existing cap structure analog Cleancap, the oligonucleotide primer with an acyclic nucleoside structure for initial capping of the present disclosure has higher synthesis efficiency, higher capping efficiency, lower immunogenicity, and higher protein translation efficiency.

### Definition of Terms

Unless otherwise defined, all technical and scientific terms used herein have the same meaning as commonly understood by one of ordinary skill in the art.

Some terms are defined as follows:

“Alkyl” when used as a group or part of a group means a C₁-C₂₀ linear or branched aliphatic hydrocarbon group, preferably a C₁-C₁₀ alkyl group, more preferably a C₁-C₆ alkyl group, or a C₁-C₄ alkyl group. Examples of alkyl groups include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl -2-m ethylpropyl, 1,1,2-tri methylpropyl, 1,1-dimethylbutyl, 1,2-dimethylbutyl, 2,2-dimethylbutyl, 1,3 -dimethylbutyl, 2-ethylbutyl, 2-methylpentyl, 3-methylpentyl, 4-methylpentyl, 2,3-dimethylbutyl, etc. Alkyl groups may be substituted or unsubstituted.

“O-alkyl” refers to the group (alkyl-O-), and the alkyl is as defined herein. C₁-C₆ and C₁-C₄ are preferred. Examples of O-alkyl include, but are not limited to: methoxy, ethoxy, n-propoxy, isopropoxy, n-butoxy, isobutoxy, tert-butoxy, etc. O-alkyl groups may be substituted or unsubstituted.

“Aryl” refers to a carbocyclic aromatic system containing one or two rings, wherein the rings may be linked together in a fused manner. The term “aryl” includes monocyclic or bicyclic aryl groups, such as phenyl, naphthyl, and tetrahydronaphthyl. Preferred aryl groups are C₆-C₁₀ aryl groups, and more preferred aryl groups are phenyl and naphthyl. Aryl groups may be substituted or unsubstituted.

“Halogen” refers to fluorine, chlorine, bromine and iodine.

“Substituted” means that one or more hydrogen, preferably up to 5 atoms, more preferably 1 to 3 hydrogen atoms in a group, are independently substituted by a corresponding number of substituents. It goes without saying that the substituents are only in their possible chemical positions, and the person skilled in the art is able to determine (either experimentally or theoretically) possible or impossible substitutions without undue effort.

Unless otherwise indicated, the structures described herein further include all isomers (e.g., diastereomeric, enantiomeric, atropisomeric and geometrically (conformationally) isomeric forms) of the structure; and certain salts of the compound which can maintain its original biological activity. The salt form of the compound represented by formula (I) can be a metal salt, an amine salt formed with a suitable acid, etc.

### BRIEF DESCRIPTION OF DRAWINGS

FIG. 1 shows cell phenotype of Examples 1-4 and Comparative Examples 1-2.

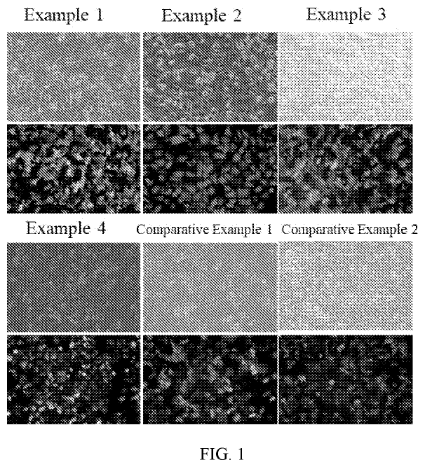

FIG. 2 shows fluorescence statistical results of Examples 1-4 and Comparative Examples 1-2.

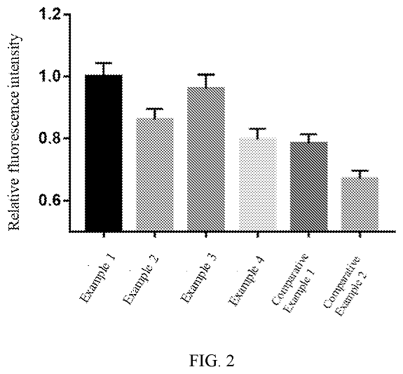

### DETAILED DESCRIPTION

The technical solution of the present disclosure will be clearly and completely described below with reference to the examples of the present disclosure. Apparently, the described examples are only some, not all, of the embodiments of the present disclosure. Based on the embodiments of the present disclosure, all other embodiments obtained by those of ordinary skill in the art without making inventive efforts fall within the protection scope of the present disclosure.

The names and sources of raw materials used in each example are shown in Table 1 below:

TABLE 1

| Reagent name | Manufacturer |
|---|---|
| Guanosine | Wuhu Huaren Co., Ltd. |
| Adenosine | Wuhu Huaren Co., Ltd. |
| Tert-butyldimethylsilyl | Anhui Zesheng Technology Co., Ltd. |
| chloride | |
| Imidazole | Anhui Zesheng Technology Co., Ltd. |
| Tetrazole | Sinopharm Chemical Reagent Co., Ltd. |
| Triphenylphosphine | Anhui Zesheng Technology Co., Ltd. |
| Disulfidepyridine | Anhui Zesheng Technology Co., Ltd. |
| Sodium periodate | Anhui Zesheng Technology Co., Ltd. |
| Sodium perchlorate | Sinopharm Chemical Reagent Co., Ltd. |
| 2′OMe-rA | Wuhu Huaren Co., Ltd. |
| phosphoramidite monomer | |
| GDP | Meiya Pharmaceutical Haian Co., Ltd. |
| DMTrCl | Anhui Zesheng Technology Co., Ltd. |
| 2′,3′acetylguanosine | Wuhu Huaren Co., Ltd. |
| DMF | Anhui Zesheng Technology Co., Ltd. |
| DCM | Taizhou Meilan Chemical Co., Ltd. |
| Petroleum ether | Sinopharm Chemical Reagent Co., Ltd. |
| Methanol | Sinopharm Chemical Reagent Co., Ltd. |
| Concentrated ammonia | Shanghai Aladdin Biochemical |
| | Technology Co., Ltd. |
| Acetonitrile | Anhui Zesheng Technology Co., Ltd. |
| Triethylamine | Shanghai Aladdin Biochemical |
| | Technology Co., Ltd. |
| Anhydrous methanol | Anhui Zesheng Technology Co., Ltd. |
| Hydrochloric acid | Sinopharm Chemical Reagent Co., Ltd. |
| Acetone | Sinopharm Chemical Reagent Co., Ltd. |
| Ammonium acetate | Anhui Zesheng Technology Co., Ltd. |
| Trimethylphosphate | Anhui Zesheng Technology Co., Ltd. |
| Tributylamine phosphate | Wuhu Huaren Co., Ltd. |
| Dimethyl sulfate | Anhui Zesheng Technology Co., Ltd. |
| Phosphine reagent | Bengbu Nako Chemical Co., Ltd. |
| 102691-36-1 | |
| Sodium borohydride | Anhui Zesheng Technology Co., Ltd. |
| Sodium hydroxide | Shanghai Aladdin Biochemical |
| | Technology Co., Ltd. |
| Phosphorus oxychloride | Anhui Zesheng Technology Co., Ltd. |
| Acetic anhydride | Sinopharm Chemical Reagent Co., Ltd. |
| Iodine pyridine solution | Shanghai Aladdin Biochemical |
| | Technology Co., Ltd. |
| Trifluoroacetate | Shanghai Aladdin Biochemical |
| | Technology Co., Ltd. |
| TMSCl | Anhui Zesheng Technology Co., Ltd. |
| NaH | Sinopharm Chemical Reagent Co., Ltd. |
| Methyl iodide | Anhui Zesheng Technology Co., Ltd. |
| TBAF | Shanghai Aladdin Biochemical |
| | Technology Co., Ltd. |
| MnCl₂ | Shanghai Aladdin Biochemical |
| | Technology Co., Ltd. |

The m7UrGDP-Im(J) used in the following examples was prepared through the following steps:

(1) 5 g of guanosine was weighed and dispersed in 50 mL of DMF. The reaction system was subjected to an ice bath to control the internal temperature of the reaction solution below 10° C. 1.2 eq of TB SC1 was added in two batches, and the reaction was monitored by HPLC until the raw material was ≤5%. After the reaction was completed, the product was precipitated with 100 mL of water and then filtered, and the filter cake was washed to obtain target compound B.

(2) 2 g of compound B was weighed and dispersed in 20 mL of acetonitrile, and 1.2 eq of sodium periodate was added. The reaction was heated to 50° C., and monitored after 12 h. After the reaction was completed, the reaction solution was added with 40 mL of water and filtered to obtain target compound C.

(3) 2 g of compound C was weighed and dissolved in anhydrous methanol. The reaction solution was cooled to 4° C., added with 5 eq of sodium borohydride, and stirred thoroughly. The reaction was monitored by HPLC, and ice water was slowly added to quench the reaction after the reaction was completed. The reaction mixture was concentrated to dryness to obtain compound D, which was then dissolved in water. The pH was adjusted to 3 with 2M hydrochloric acid, and the reaction solution was purified by reverse chromatography to obtain target compound E.

(4) 2 g of compound E was dissolved in 10 ml of trimethylphosphate. The reaction

solution was cooled to 0° C., and slowly dropwise added with 1.2 eq of phosphorus oxychloride. After 4 hours of reaction at low temperature, 2 M ammonium acetate solution was add to quench the reaction. The reaction mixture was purified by reverse phase chromatography to obtain target compound F, which was then fully reacted with 1 eq of triphenylphosphine, 2 eq of dithiodipyridine, and 4 eq of imidazole. The reaction solution was added with 4 M sodium perchlorate acetone solution for precipitation, and the filter cake was washed thoroughly with acetone to obtain target compound G.

(5) 2 g of target compound G was weighed, dissolved in DMF, added with 3 eq of tributylamine phosphate, and stirred thoroughly to obtain target compound H. The reaction solution was added with 20 eq of aqueous solution, cooled to 4° C., and slowly added with dimethyl sulfate dropwise ester, during which the pH was adjusted to no more than 5 with 2M sodium hydroxide. The reaction was monitored by HPLC, and after the reaction was completed, purification was performed by ion chromatography to obtain target compound I.

(6) 4 g of compound I was dissolved in 50 mL of DMF, and fully reacted with 1 eq of triphenylphosphine, 2 eq of dithiodipyridine, and 4 eq of imidazole. The reaction solution was added with 4 M sodium perchlorate acetone solution for precipitation, and the filter cake was washed thoroughly with acetone to obtain target compound J.

The specific reaction route process of m7UrGDP-Im(J) is shown in the following equation (1):

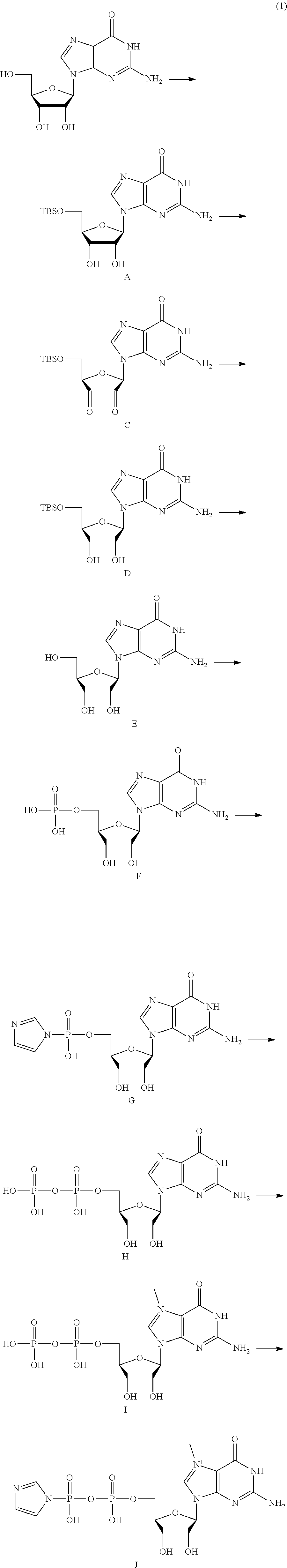

The synthetic route of A-G-P used in Example 1 is as follows. 5 kg of 2′OMe-rA phosphoramidite monomer was weighed, placed in a one-neck bottle, dissolved with 50 L of dichloromethane, then added with 2.73 kg of 2′,3′acetylguanosine, cooled to 25±2° C., added with 880 g of tetrazole under nitrogen blowing, and heated to 25±2° C. for reaction. After the reaction was monitored to be completed, 1.2 eq of iodopyridine solution was added to the reaction solution. After the reaction was monitored to be completed, the reaction solution was spun to dryness, and the concentrated ointment was dissolved in 4 L of dichloromethane and added with 1.1 eq of trifluoroacetic acid. After the reaction was monitored to be completed, the reaction solution was spun to dryness, slurried with petroleum ether/dichloromethane in a certain ratio, and filtered to obtain intermediate A2. A2 was dissolved in 4 L of acetonitrile, added with 1.2 eq of phosphine reagent and 1.2 eq of tetrazole, and stirred thoroughly. After the reaction was monitored to be completed, 1.2 eq of iodopyridine solution was added to the reaction solution. After the reaction was monitored to be completed, the reaction solution was spun to dryness, and 3 L of methanol and 3 L of concentrated ammonia were added in the spin bottle for 4 h of reaction at room temperature. After the reaction was monitored to be completed, the reaction solution was spun to dryness, added with 20 L of ultrapure water, introduced to the reverse ion permeation equipment, washed, concentrated, and freeze-dried to obtain the target compound A-G-P. The reaction route is shown in the following equation (2):

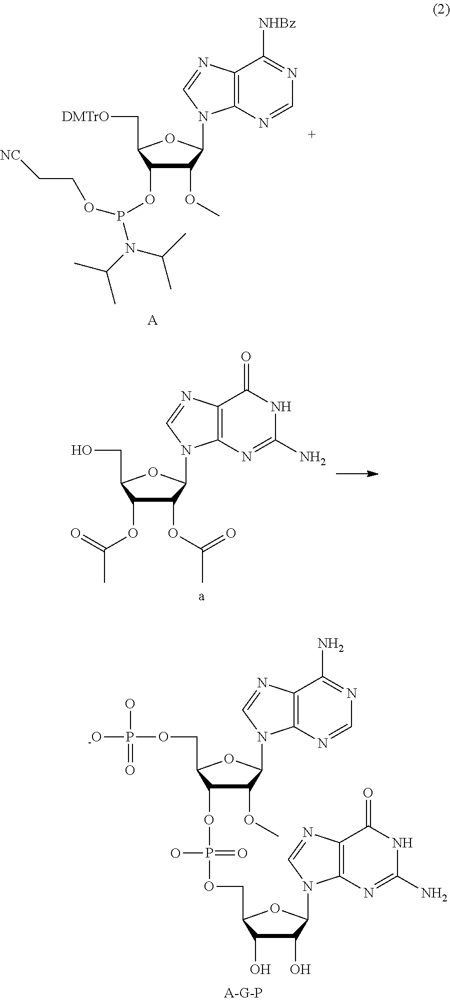

The synthesis method of A-UrG-P used in Example 2 refers to the synthesis method of A-G-P in Example 1, and the reaction route of A-UrG-P is shown in the following equation (3):

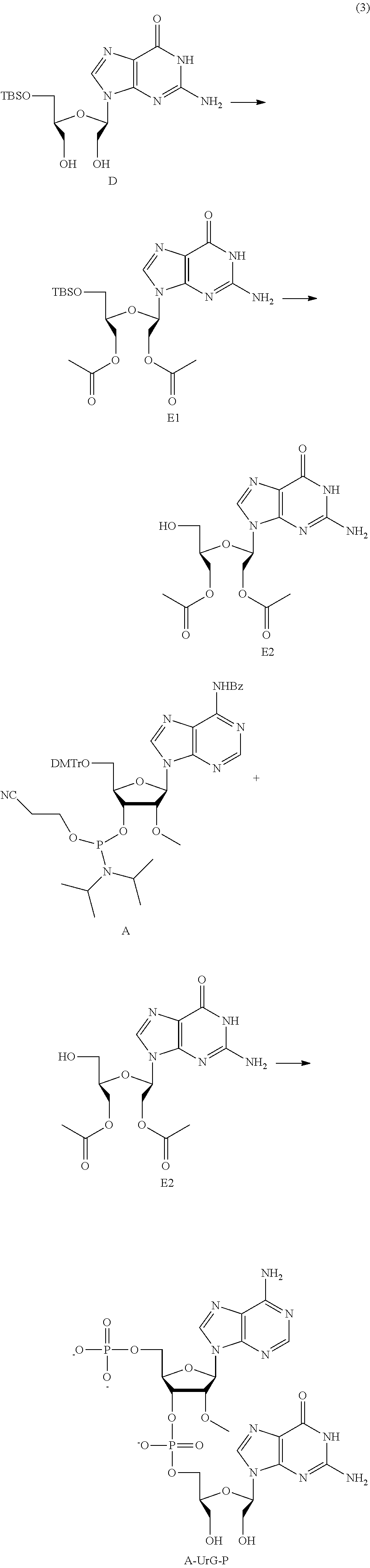

The preparation of E2 includes the following steps: 20 g of compound D was weighed, dissolved in acetonitrile, and added with 3 eq of triethylamine. The reaction solution was cooled to 4° C. and slowly added with acetic anhydride dropwise. After the reaction was completed, 2 eq of TBAF was added to remove TBS protection group, and the reaction solution was spun to dryness and subjected to column chromatography to obtain compound E2. Compound E2 was substituted with disubstituted guanosine to obtain A-UrG-P.

The synthesis method of UrA-G-P used in Example 3 refers to the synthesis method of A-G-P in Example 1, and the reaction route of UrA-G-P is shown in the following equation (4):

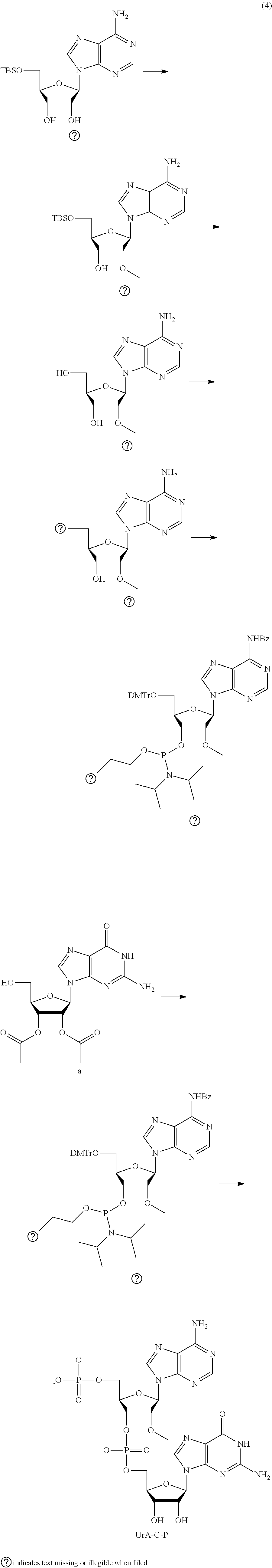

The synthesis of D refers to the synthesis steps of intermediate J. The preparation of F4 includes the following steps: (1) 10 g of compound D was weighed, dissolved in D1VIF, subjected to an ice bath, slowly added with 1.2 eq of NaH, stirred at low temperature for 2 h, and slowly added 2 eq of methyl iodide dropwise. The reaction was performed at room temperature for three hours, and then quenched with water. The reaction mixture was filtered to obtain the crude product of compound F1, which was then purified by reverse chromatography. (2) 2 g of compound F was weighed, dispersed in 30 mL of methanol, and added with 2 eq of TBAF. After the reaction was completed in 2 hours, the reaction solution was spun to dryness directly for the next step. The spin-dried solid was dissolved in 30 ml of DCM, added with 1.2 eq of triethylamine, stirred in an ice bath for 20 min, and slowly added with DCM solution of DMTr-Cl. The reaction was performed for half an hour after the dropwise addition was completed. Column chromatography was performed to obtain target compound F3. (3) 3 g of compound F3 was weighed, and subjected to transfer protection with 2 eq of TMSC1 by protecting the amino group with Bz at the 6′ position. After purification, it was reacted with tetrazole and phosphine reagent to obtain target compound F4. Compound F4 was substituted with 2′OMe-rA phosphoramidite monomer to obtain UrA-G-P.

The synthesis method of UrA-UrG-P used in Example 4 refers to the synthesis method of A-G-P in Example 1. UrA-UrG-P was obtained through the reaction of E2 and F4. The reaction route is shown in the following equation (5):

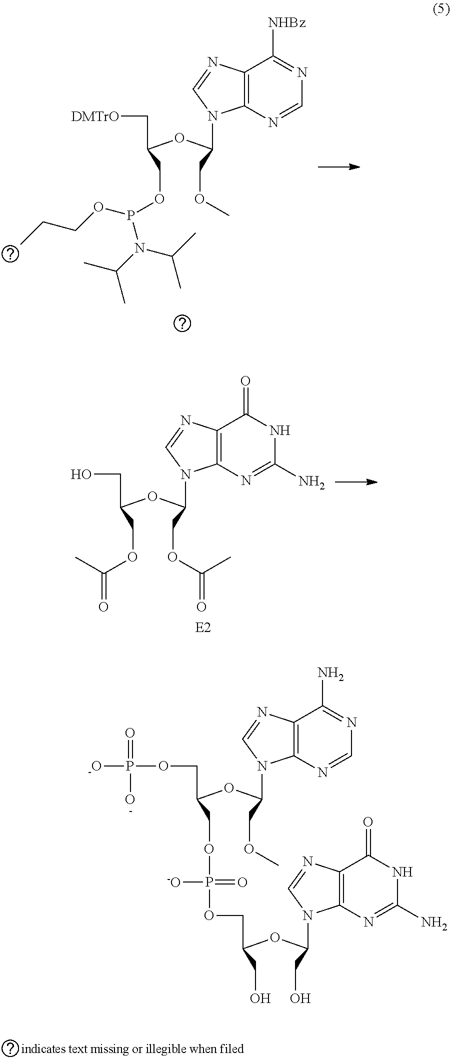

Example 1: Synthesis method of an oligonucleotide primer with an acyclic nucleoside structure for initial capping in which both Ra and Rb are five-membered sugar rings

It was synthesized through the following steps using m7UrGDP-Im(J) and A-G-P as raw materials: m7UrGDP-Im(J) (2 mol) was dissolved in a DMF solution containing MnCl₂ (0.2 mol), added to the D1VIF solution of A-G-P (1.8 mol), and stirred at room temperature. After 24 h, the reaction was terminated with 10 L of 0.25 M EDTA solution. The mixture was loaded onto a DEAE Sephadex column (30×500 cm). The product was eluted using a linear gradient of 0-1.0 M TEAB eluent. The eluted product with HPLC purity >97% was collected. The above separated liquid was concentrated, then loaded onto a strong anionic resin, and eluted using a linear gradient of 0-1.0 M sodium acetate eluent. The eluted product with HPLC purity >98.5% was collected, and the high-purity eluates were combined. The residual sodium acetate solution was removed through nanofiltration equipment. After concentration, the target product was obtained. The reaction route is shown in the following equation (6):

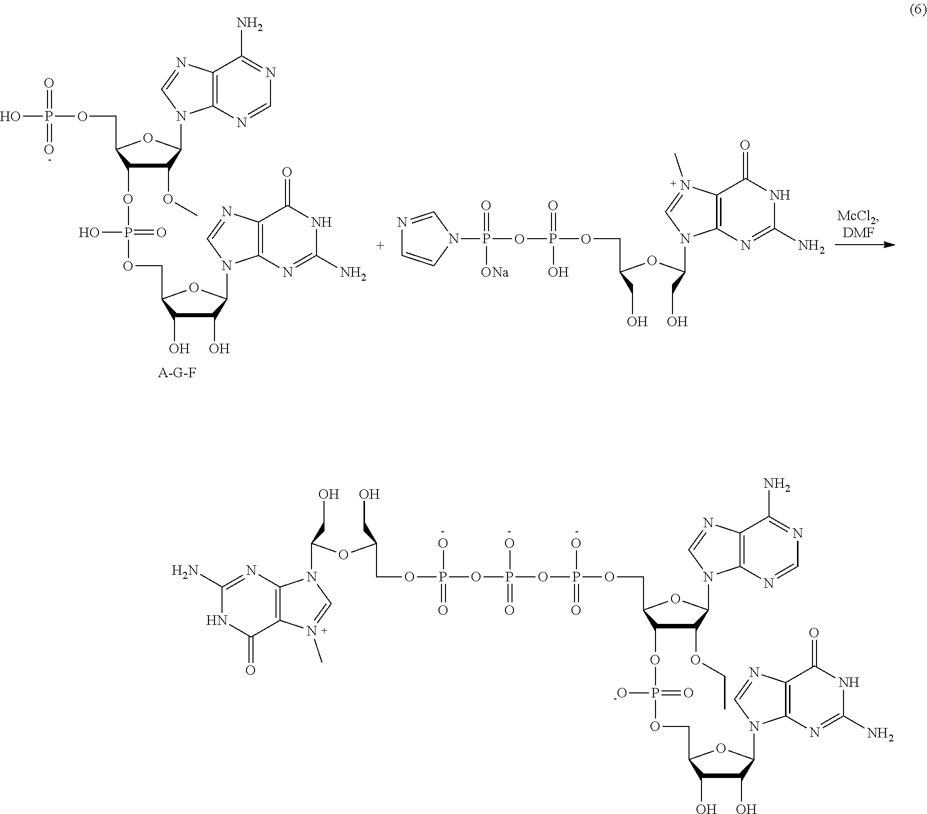

Example 2: Oligonucleotide primer with an acyclic nucleoside structure for initial capping in which Ra is a five-membered sugar ring and Rb is a acyclic structure

The oligonucleotide primer with an acyclic nucleoside structure for initial capping in this example was prepared using m7UrGDP-Im(J) and A-UrG-P as raw materials by referring to the synthesis method of the target product in Example 1.

Example 3: Oligonucleotide primer with an acyclic nucleoside structure for initial capping in which Ra has a acyclic structure and Rb is a five-membered sugar ring

The oligonucleotide primer with an acyclic nucleoside structure for initial capping in this example was prepared using m7UrGDP-Im(J) and UrA-G-P as raw materials by referring to the synthesis method of the target product in Example 1.

Example 4: Oligonucleotide primer with an acyclic nucleoside structure for initial capping in which both Ra and Rb have acyclic structures

The oligonucleotide primer with an acyclic nucleoside structure for initial capping in this example was prepared using m7UrGDP-Im(J) and UrA-UrG-P as raw materials by referring to the synthesis method of the target product in Example 1.

Example 5: Synthesis method of an oligonucleotide primer with an acyclic nucleoside structure for initial capping using intermediates J and K as raw materials

The oligonucleotide primer for initial capping in Example 5 was prepared using intermediates J and K as raw materials by referring to the synthesis method of the target product in Example 1. The reaction route is shown in the following equation (6):

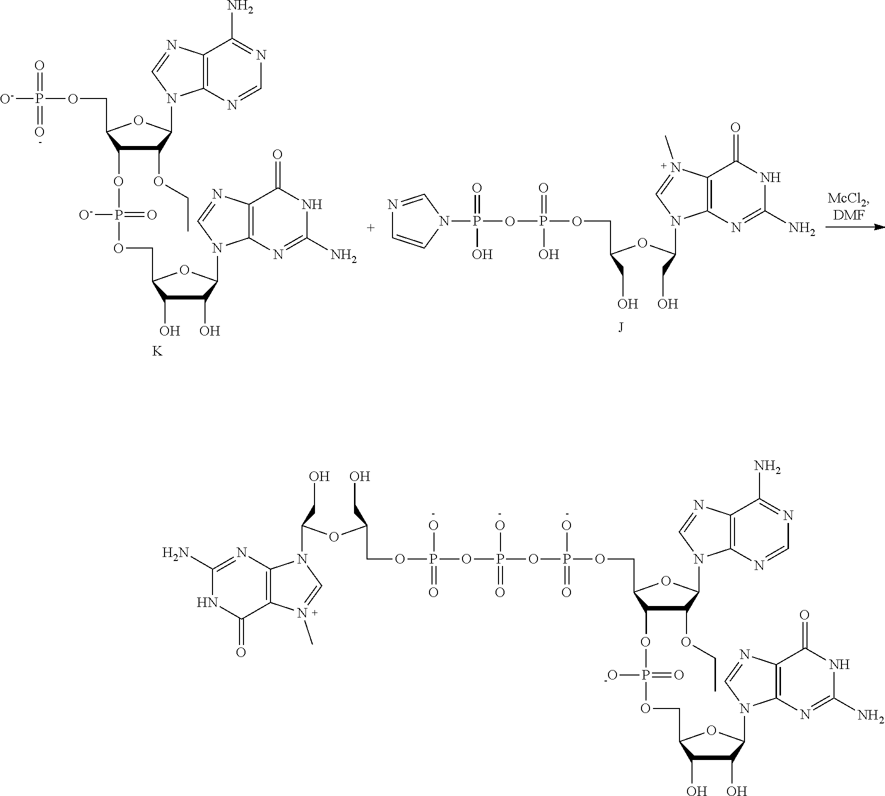

Wherein, compound K was prepared through the following steps:

(1) 200.0 g of 2′OEt-rA phosphoramidite monomer and N²-isobutyryl-2′,3′-acetylguanosine (1.0 eq) were weighed, placed in a one-neck bottle, dissolved with 2.0 L of dichloromethane, and added with tetrazole (2.1 eq) under nitrogen blowing for 3 h of reaction at 25° C. After the reaction was monitored to be completed, 70% tert-butyl hydroperoxide aqueous solution (3.0 eq) was added dropwise to the reaction solution for 1 h of reaction at 25° C. After the reaction was monitored to be completed, trichloroacetic acid (4.0 eq) in the dichloromethane solution was added dropwise to the reaction solution for 1 h of reaction at room temperature. After the reaction was monitored to be completed, the reaction solution was washed with 10% sodium sulfite aqueous solution, 10% sodium bicarbonate aqueous solution, and saturated brine. The organic phase was concentrated and purified by column chromatography to obtain intermediate K1.

(2) Intermediate K1 was dissolved in acetonitrile (10V), added with 1.8 eq of bis(2-cyanoethyl)-N,N-diisopropylphosphoramidite and 1.8 eq of tetrazole, and stirred at room temperature for 2 h under nitrogen atmosphere. After the reaction was completed, 70% tert-butyl hydroperoxide aqueous solution (3.0 eq) was added dropwise to the reaction solution for 1 h of reaction at room temperature. After the reaction was monitored to be completed, the reaction solution was spun to dryness, and methanol and concentrated ammonia (10y, 1:1) were added in the spin bottle for 14 h of reaction at room temperature. After the reaction was monitored to be completed, the reaction solution was spun to dryness. The crude product was purified by ion chromatography and concentrated to obtain intermediate K. The reaction route is shown in the following equation (7):

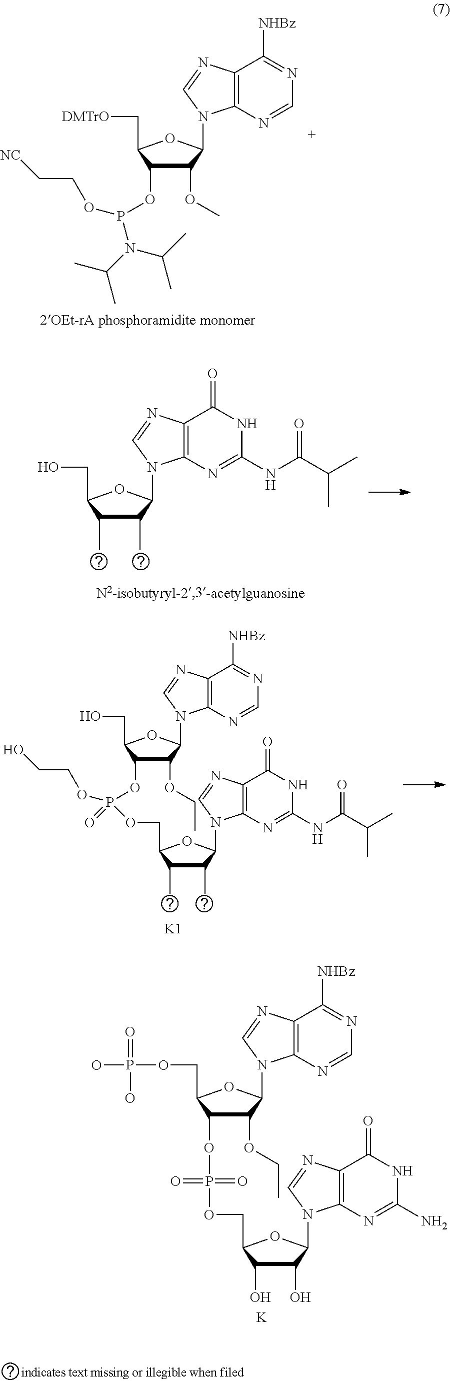

Example 6: Synthesis method of an oligonucleotide primer with an acyclic nucleoside structure for initial capping using intermediates J and L as raw materials

The oligonucleotide primer for initial capping in Example 6 was prepared using intermediates J and L as raw materials by referring to the synthesis method of the target product in Example 1. The reaction route is shown in the following equation (8):

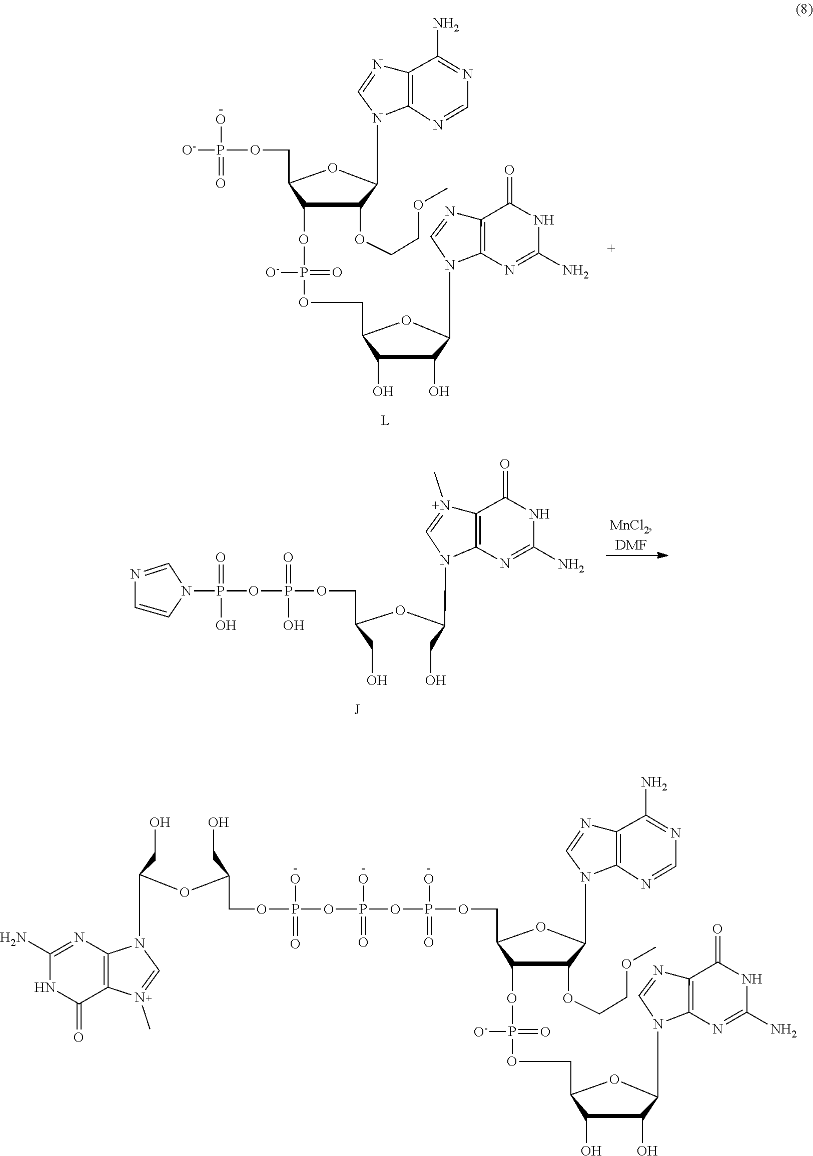

Wherein, compound L was prepared through the following steps:

(1) 200.0 g of 2′O-MOE-rA phosphoramidite monomer and N² -isobutyryl-2′,3′-acetylguanosine (1.0 eq) were weighed, placed in a one-neck bottle, dissolved with 2.0 L of dichloromethane, and added with tetrazole (2.1 eq) under nitrogen blowing for 3 h of reaction at 25° C. After the reaction was monitored to be completed, an aqueous solution of 70% tert-butyl hydroperoxide (3.0 eq) was added dropwise to the reaction solution for 1 h of reaction at 25° C. After the reaction was monitored to be completed, a solution of trichloroacetic acid (4.0 eq) in dichloromethane was added dropwise to the reaction solution for 1 h of reaction at room temperature. After the reaction was monitored to be completed, the reaction solution was washed with 10% sodium sulfite aqueous solution, 10% sodium bicarbonate aqueous solution, and saturated brine. The organic phase was concentrated and purified by column chromatography to obtain intermediate L1.

(2) Intermediate L1 was dissolved in acetonitrile (10V), added with 1.8 eq of bis(2-cyanoethyl)-N,N-diisopropylphosphoramidite and 1.8 eq of tetrazole, and stirred at room temperature for 2 h under nitrogen atmosphere. After the reaction was completed, 70% tert-butyl hydroperoxide aqueous solution (3.0 eq) was added dropwise to the reaction solution for 1 h of reaction at room temperature. After the reaction was monitored to be completed, the reaction solution was spun to dryness, and methanol and concentrated ammonia (10V, 1:1) were added in the spin bottle for 14 h of reaction at room temperature. After the reaction was monitored to be completed, the reaction solution was spun to dryness. The crude product was purified by ion chromatography and concentrated to obtain intermediate L. The reaction route is shown in the following equation (9):

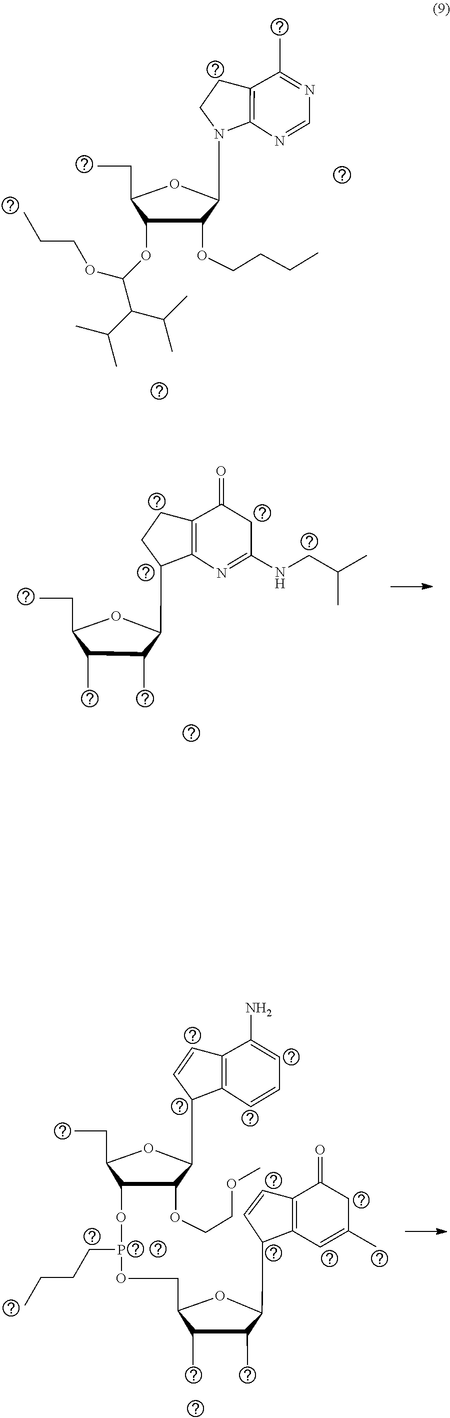

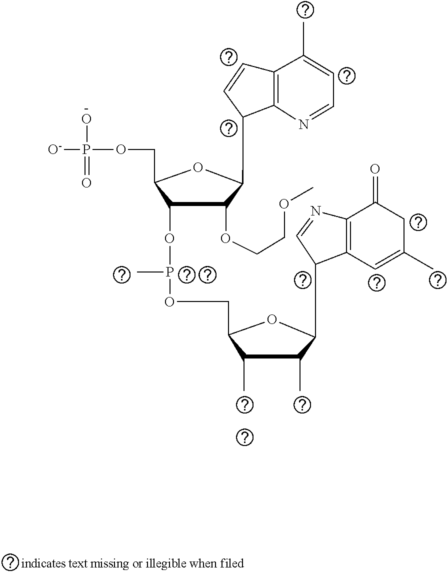

Example 7: Synthesis method of an oligonucleotide primer with an acyclic nucleoside structure for initial capping using intermediates J and M as raw materials

The oligonucleotide primer for initial capping in Example 7 was prepared using intermediates J and M as raw materials by referring to the synthesis method of the target product in Example 1. The reaction route is shown in the following equation (10):

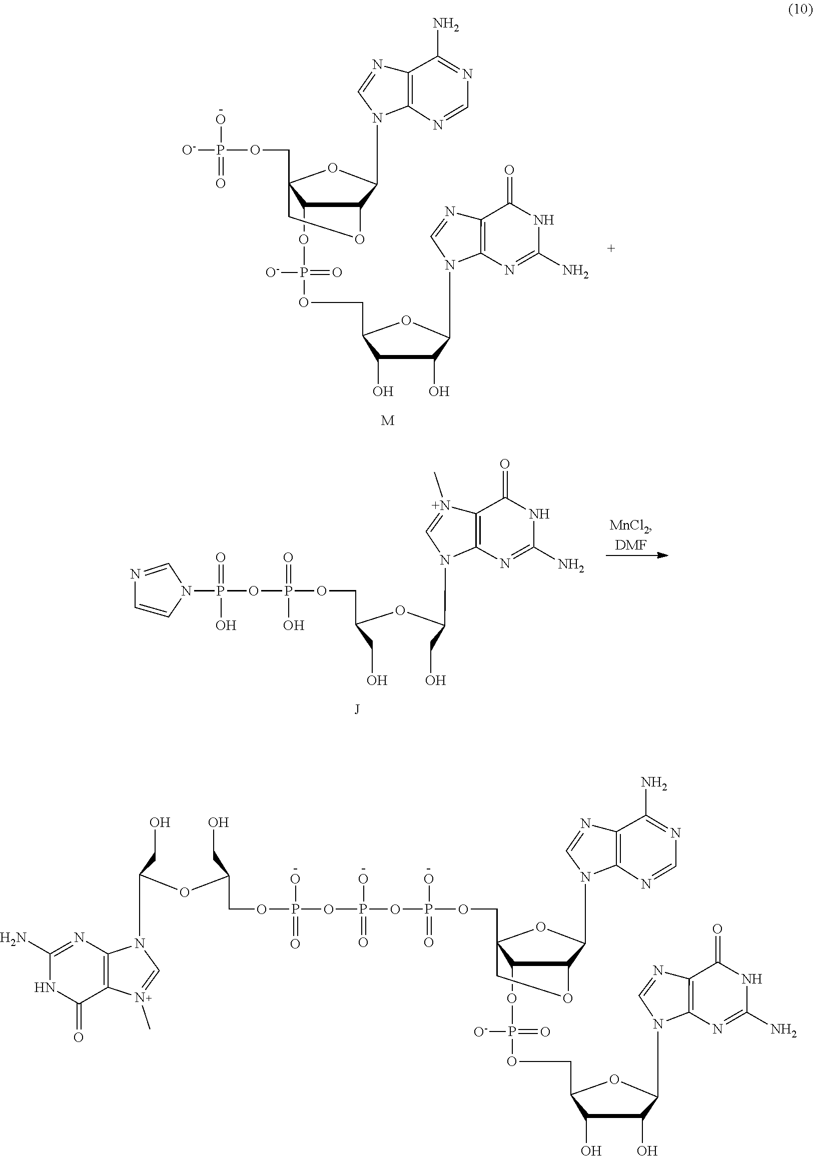

Wherein, compound M was prepared through the following steps:

(1) 200.0 g of 2′,4′-ring-locked-rA phosphoramidite monomer and N²-isobutyryl-2′,3′-acetylguanosine (1.0 eq) were weighed, placed in a one-neck bottle, dissolved with 2.0 L of dichloromethane, and added with tetrazole (2.1 eq) under nitrogen blowing for 3 h of reaction at 25° C. After the reaction was monitored to be completed, 70% tert-butyl hydroperoxide aqueous solution (3.0 eq) was added dropwise to the reaction solution for 1 h of reaction at 25° C. After the reaction was monitored to be completed, trichloroacetic acid (4.0 eq) in the dichloromethane solution was added dropwise to the reaction solution for 1 h of reaction at room temperature. After the reaction was monitored to be completed, the reaction solution was washed with 10% sodium sulfite aqueous solution, 10% sodium bicarbonate aqueous solution, and saturated brine. The organic phase was concentrated and purified by column chromatography to obtain intermediate M1

(2) Intermediate M1 was dissolved in acetonitrile (10V), added with 1.8 eq of bis(2-cyanoethyl)-N,N-diisopropylphosphoramidite and 1.8 eq of tetrazole, and stirred at room temperature for 2 h under nitrogen atmosphere. After the reaction was completed, 70% tert-butyl hydroperoxide aqueous solution (3.0 eq) was added dropwise to the reaction solution for 1 h of reaction at room temperature. After the reaction was monitored to be completed, the reaction solution was spun to dryness, and methanol and concentrated ammonia (10V, 1:1) were added in the spin bottle for 14 h of reaction at room temperature. After the reaction was monitored to be completed, the reaction solution was spun to dryness. The crude product was purified by ion chromatography and concentrated to obtain intermediate M. The reaction route is shown in the following equation (11):

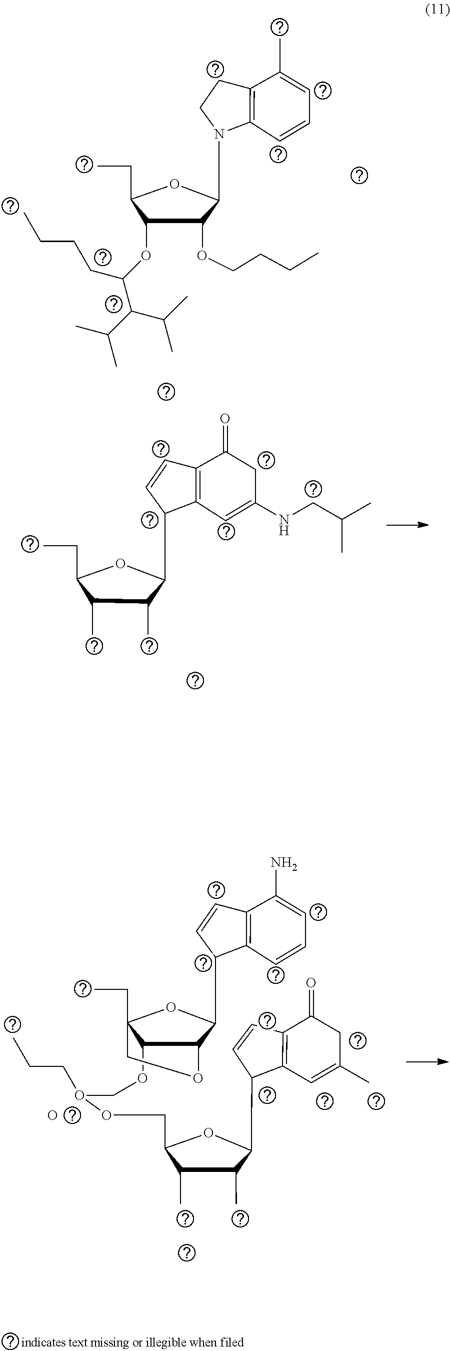

Example 8: Synthesis method of an oligonucleotide primer with an acyclic nucleoside structure for initial capping using intermediates J and N as raw materials

The oligonucleotide primer for initial capping in Example 8 was prepared using intermediates J and N as raw materials by referring to the synthesis method of the target product in Example 1. The reaction route is shown in the following equation (12):

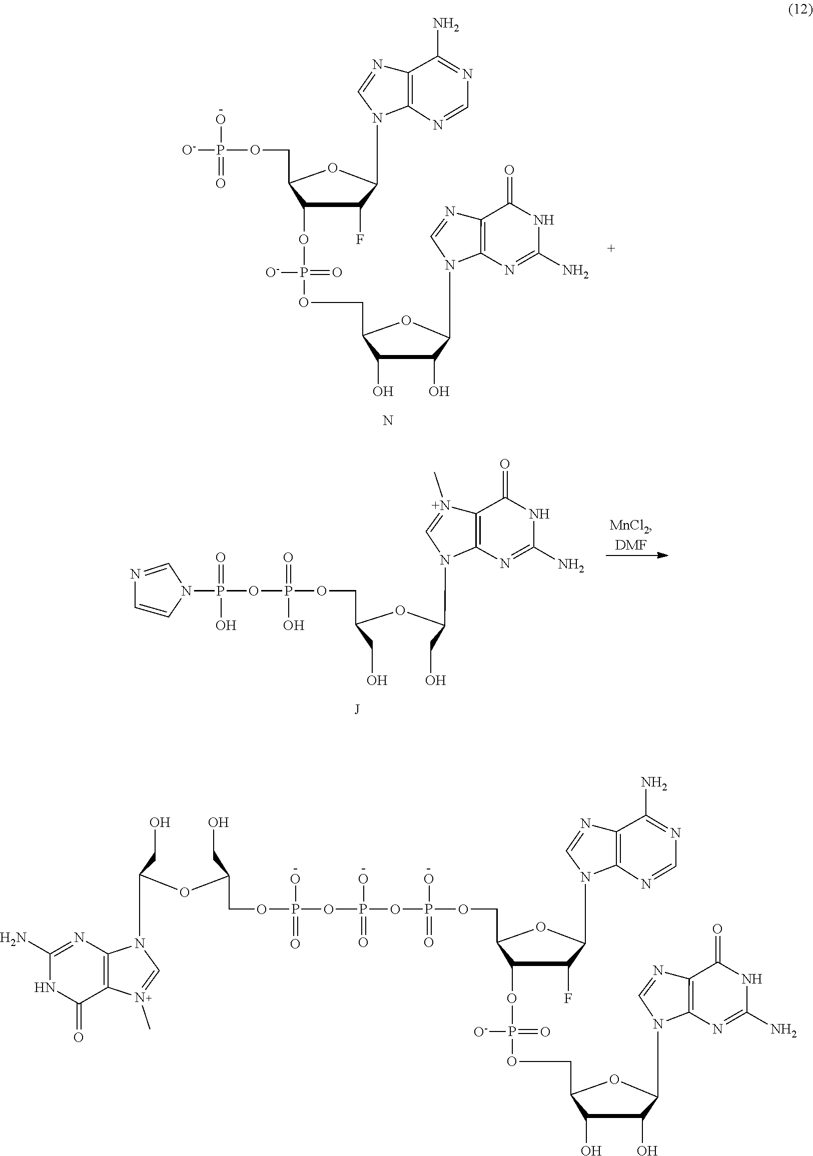

Wherein, compound N was prepared through the following steps:

(1) 200.0 g of 2′-F-rA phosphoramidite monomer and N²-isobutyryl-2′,3′-acetylguanosine (1.0 eq) were weighed, placed in a one-neck bottle, dissolved with 2.0 L of dichloromethane, and added with tetrazole (2.1 eq) under nitrogen blowing for 3 h of reaction at 25° C. After the reaction was monitored to be completed, 70% tert-butyl hydroperoxide aqueous solution (3.0 eq) was added dropwise to the reaction solution for 1 h of reaction at 25° C. After the reaction was monitored to be completed, trichloroacetic acid (4.0 eq) in dichloromethane solution was added dropwise to the reaction solution for 1 h of reaction at room temperature. After the reaction was monitored to be completed, the reaction solution was washed with 10% sodium sulfite aqueous solution, 10% sodium bicarbonate aqueous solution, and saturated brine. The organic phase was concentrated and purified by column chromatography to obtain intermediate N1.

(2) Intermediate N1 was dissolved in acetonitrile (10V), added with 1.8 eq of bis(2-cyanoethyl)-N,N-diisopropylphosphoramidite and 1.8 eq of tetrazole, and stirred at room temperature for 2 h under nitrogen atmosphere. After the reaction was completed, 70% tert-butyl hydroperoxide aqueous solution (4.0 eq) was added dropwise to the reaction solution for 1 h of reaction at room temperature. After the reaction was monitored to be completed, the reaction solution was spun to dryness, and methanol and concentrated ammonia (10V, 1:1) were added in the spin bottle for 14 h of reaction at room temperature. After the reaction was monitored to be completed, the reaction solution was spun to dryness. The crude product was purified by ion chromatography and concentrated to obtain intermediate N. The reaction route is shown in the following equation (13):

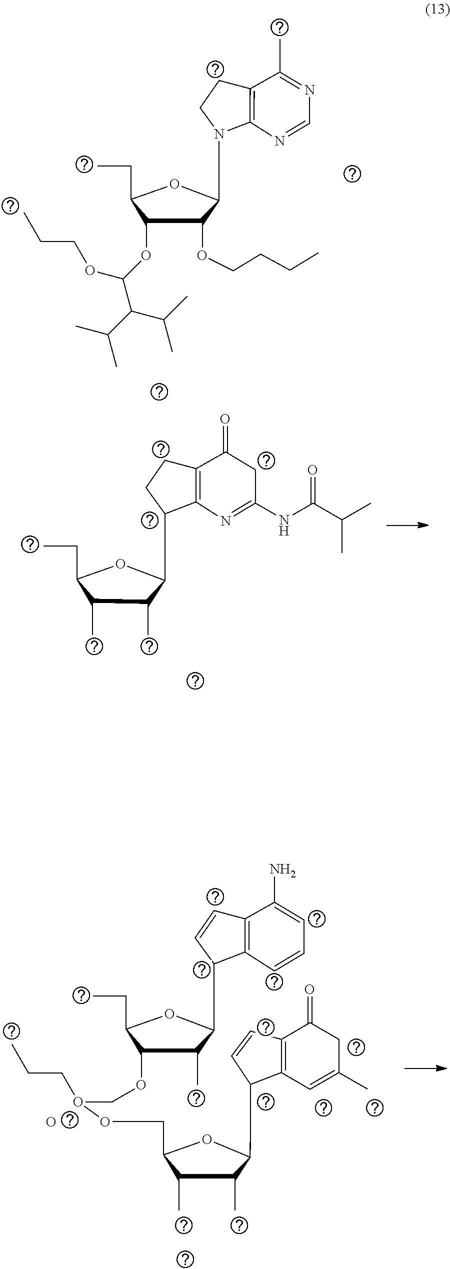

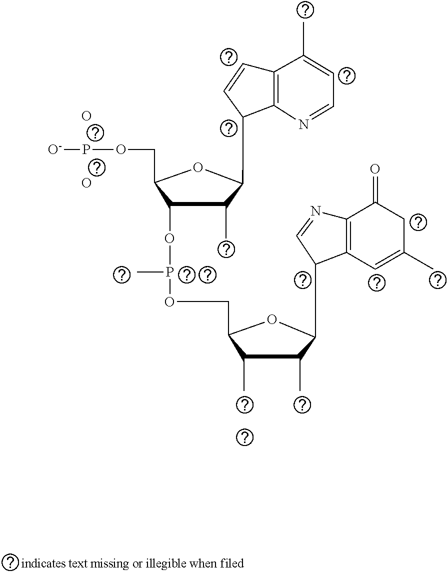

Example 9: Synthesis method of an oligonucleotide primer with an acyclic nucleoside structure for initial capping using intermediates J and O as raw materials

The oligonucleotide primer for initial capping in Example 9 was prepared using intermediates J and O as raw materials by referring to the synthesis method of the target product in Example 1. The reaction route is shown in the following equation (14):

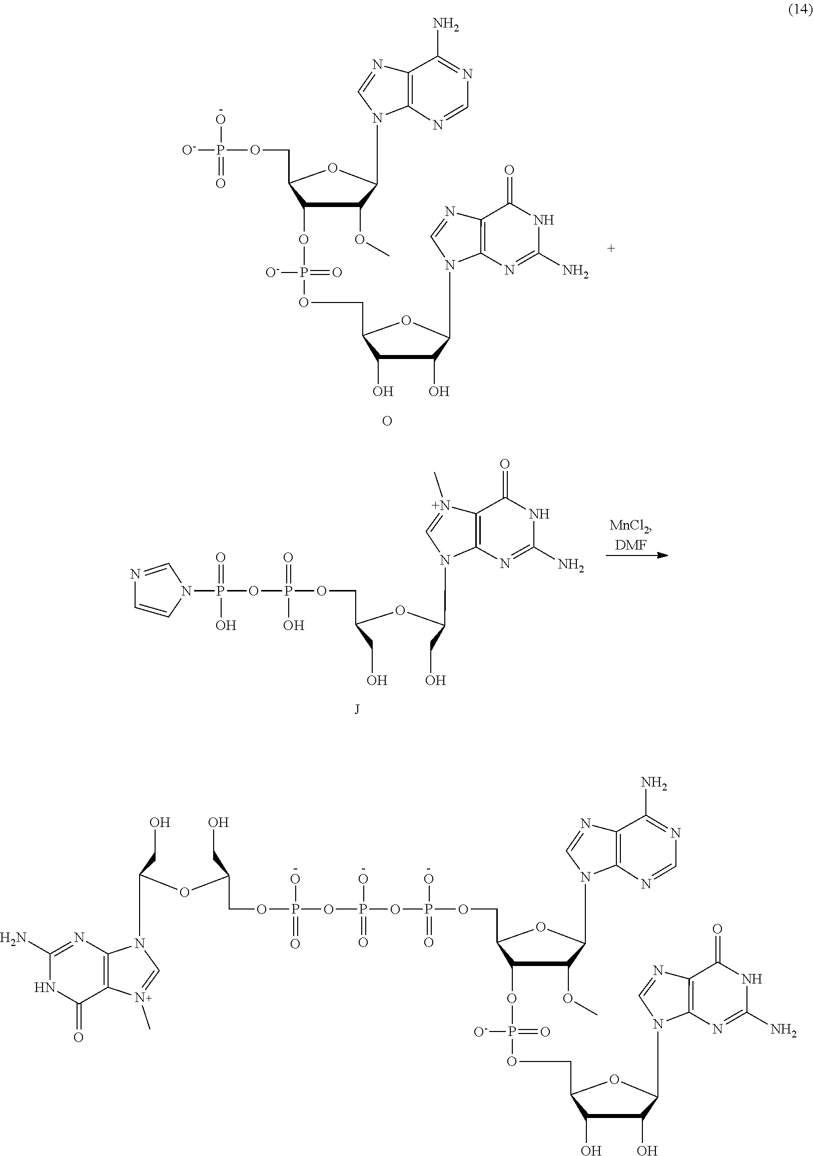

Wherein, compound O was prepared through the following steps:

(1) 200.0 g of 2′-OMe-rA phosphoramidite monomer and N²-isobutyryl-2′,3′-acetylguanosine (1.0 eq) were weighed, placed in a one-neck bottle, dissolved with 2.0 L of dichloromethane, and added with tetrazole (2.1 eq) under nitrogen blowing for 3 h of reaction at 25° C. After the reaction was monitored to be completed, hydrogenated xanthogen (2.5 eq) was added to the reaction solution for 2 h of reaction at 25° C. After the reaction was monitored to be completed, trichloroacetic acid (4.0 eq) in dichloromethane solution was added dropwise to the reaction solution for 1 h of reaction at room temperature. After the reaction was monitored to be completed, the reaction solution was washed with 10% sodium sulfite aqueous solution, 10% sodium bicarbonate aqueous solution, and saturated brine. The organic phase was concentrated and purified by column chromatography to obtain intermediate O1.

(2) Intermediate O1 was dissolved in acetonitrile (10V), added with 1.8 eq of bis(2-cyanoethyl)-N,N-diisopropylphosphoramidite and 1.8 eq of tetrazole, and stirred at room temperature for 2 h under nitrogen atmosphere. After the reaction was completed, 70% tert-butyl hydroperoxide aqueous solution (4.0 eq) was added dropwise to the reaction solution for 1 h of reaction at room temperature. After the reaction was monitored to be completed, the reaction solution was spun to dryness, and methanol and concentrated ammonia (10V, 1:1) were added in the spin bottle for 14 h of reaction at room temperature. After the reaction was monitored to be completed, the reaction solution was spun to dryness. The crude product was purified by ion chromatography and concentrated to obtain intermediate O. The reaction route is shown in the following equation (15):

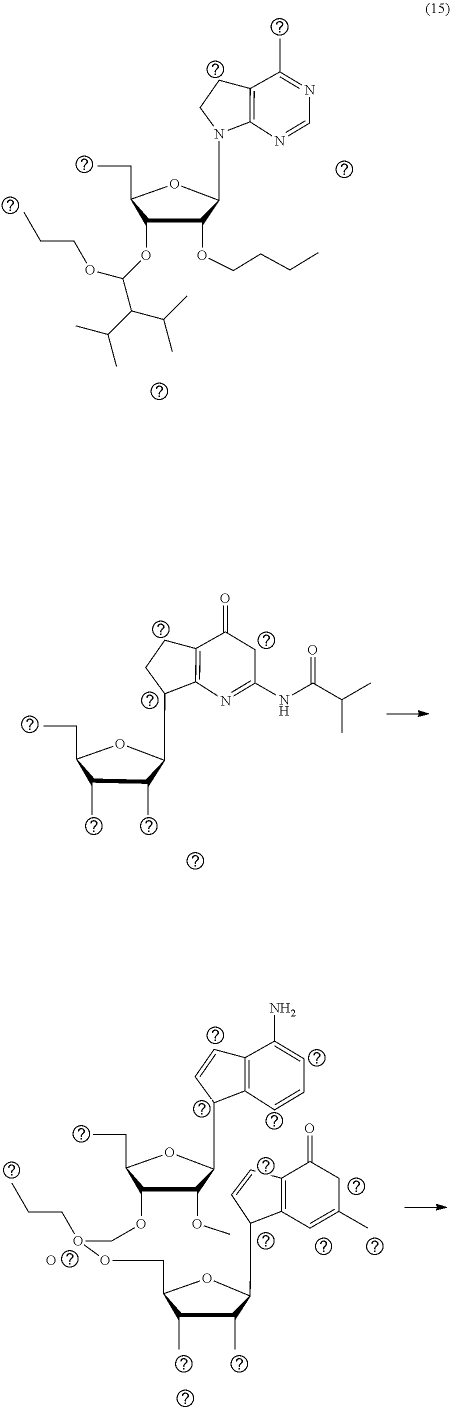

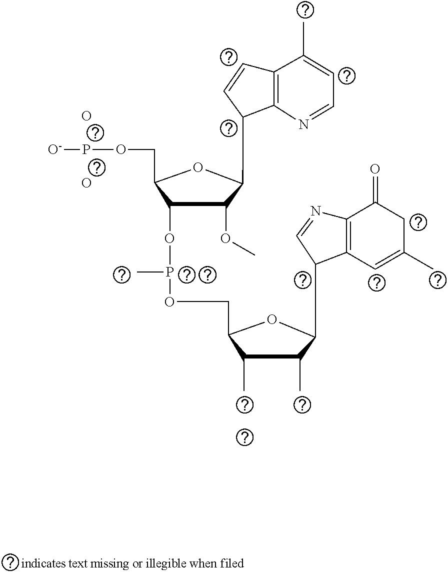

Example 10: Synthesis method of an oligonucleotide primer with an acyclic nucleoside structure for initial capping using intermediates J and P as raw materials

The oligonucleotide primer for initial capping in Example 10 was prepared using intermediates J and P as raw materials by referring to the synthesis method of the target product in Example 1. The reaction route is shown in the following equation (16):

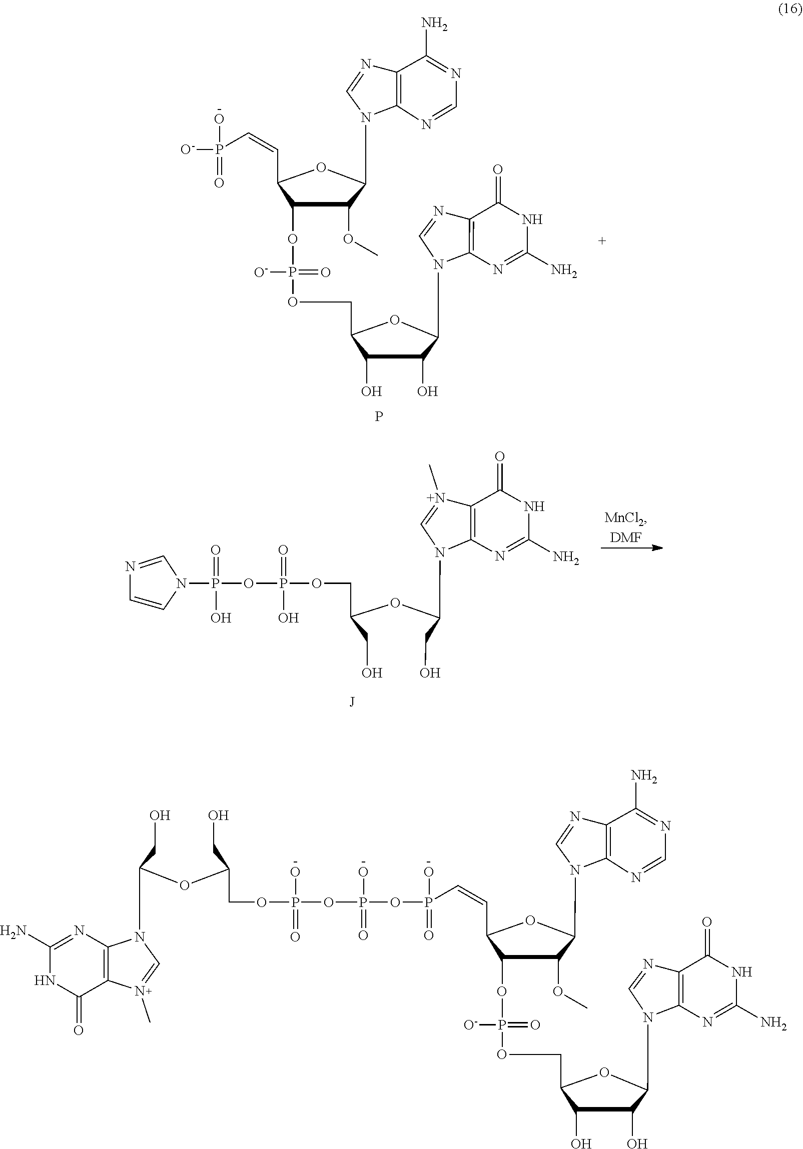

Wherein, compound P was prepared through the following steps:

(1) 50.0 g of 2′-OMe-3′-OTBS-A(Bz) was dissolved in anhydrous DMF (10V), DMSO (6.0 eq) and EDCI (3.0 eq), and then pyridine (7.5 mL) and trifluoroacetic acid (3.8 mL) in DMF (3V) solution were added dropwise to the reaction solution at room temperature. The reaction solution was stirred at room temperature for 2 h, then diluted with ethyl acetate, and washed with saturated sodium bicarbonate aqueous solution and water. The organic phase was concentrated and purified by column chromatography to obtain intermediate P1.

(2) Tetramethyl methylene diphosphate (1.5 eq) was added dropwise to a suspension of sodium hydride (60%, 1.1 eq) in tetrahydrofuran under a nitrogen atmosphere in an ice bath, and stirred at 0° C. for 0.5 h. Then, P1 in tetrahydrofuran solution was added dropwise to the above suspension and stirred at room temperature overnight. The reaction solution was quenched with saturated ammonium chloride aqueous solution and extracted with ethyl acetate. The organic phase was concentrated and purified by column chromatography to obtain intermediate P2.

(3) P2 was dissolved in anhydrous tetrahydrofuran (10V), added with tetrabutylammonium fluoride (1.0 M tetrahydrofuran solution, 2.0 eq) at room temperature and then stirred for 4 h. The reaction solution was concentrated and purified by column chromatography to obtain intermediate P3.

5 (4) Intermediate P3 was dissolved in dichloromethane, added with tetrazole (1.5 eq) and bis(diisopropylamino)(2-cyanoethoxy)phosphine (1.2 eq), and then stirred at room temperature for 3 h. After the reaction solution was washed with water, the organic phase was concentrated to obtain a crude product, which was purified by column chromatography to obtain intermediate P4.

(5) Intermediate P4 and N²-isobutyryl-2′,3′-acetylguanosine (1.0 eq) were placed in a one-neck bottle, dissolved with dichloromethane (10V), and added with tetrazole (2.1 eq) under nitrogen blowing for 3 h of reaction at 25° C. After the reaction was monitored to be completed, 70% tert-butyl hydroperoxide aqueous solution was added dropwise to the reaction solution for 2 h of reaction at 25° C. After the reaction was monitored to be completed, trichloroacetic acid (4.0 eq) in dichloromethane solution was added dropwise to the reaction solution for 1 h of reaction at room temperature. After the reaction was monitored to be completed, the reaction solution was washed with 10% sodium sulfite aqueous solution, 10% sodium bicarbonate aqueous solution, and saturated brine. The organic phase was concentrated and purified by column chromatography to obtain intermediate P5.

(6) Intermediate P5 was dissolved in acetonitrile (10V), added with 10.0 eq of trimethylsilyl bromide and stirred at room temperature under nitrogen atmosphere overnight.

After the reaction was monitored to be completed, the reaction solution was spun to dryness, and methanol and concentrated ammonia (10V, 1:1) were added in the spin bottle for 14 h of reaction at room temperature. After the reaction was monitored to be completed, the reaction solution was spun to dryness. The crude product was purified by ion chromatography and concentrated to obtain intermediate P. The reaction route is shown in the following equation (17):

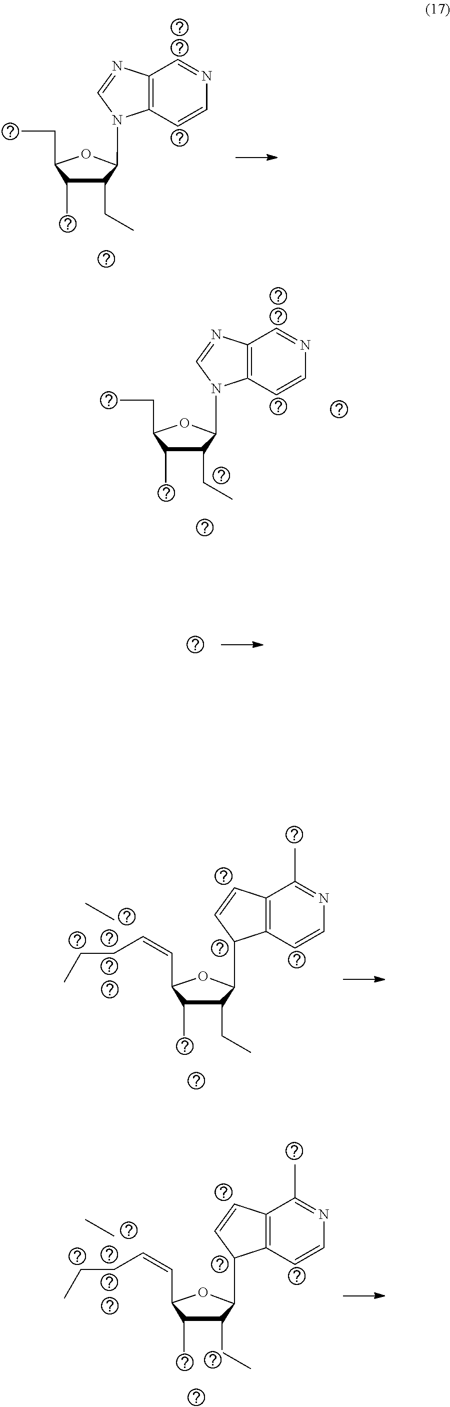

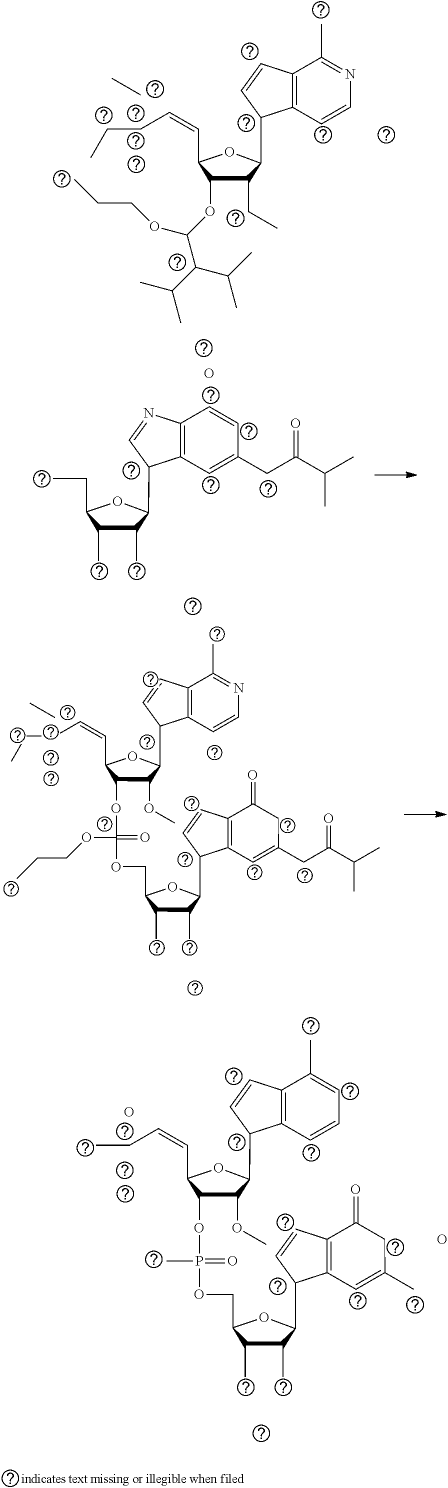

Example 11: Synthesis method of an oligonucleotide primer with an acyclic nucleoside structure for initial capping using intermediates A-G-P and Q as raw materials

The oligonucleotide primer for initial capping in Example 11 was prepared using intermediates A-G-P and Q as raw materials by referring to the synthesis method of the target product in Example 1. The reaction route is shown in the following equation (18):

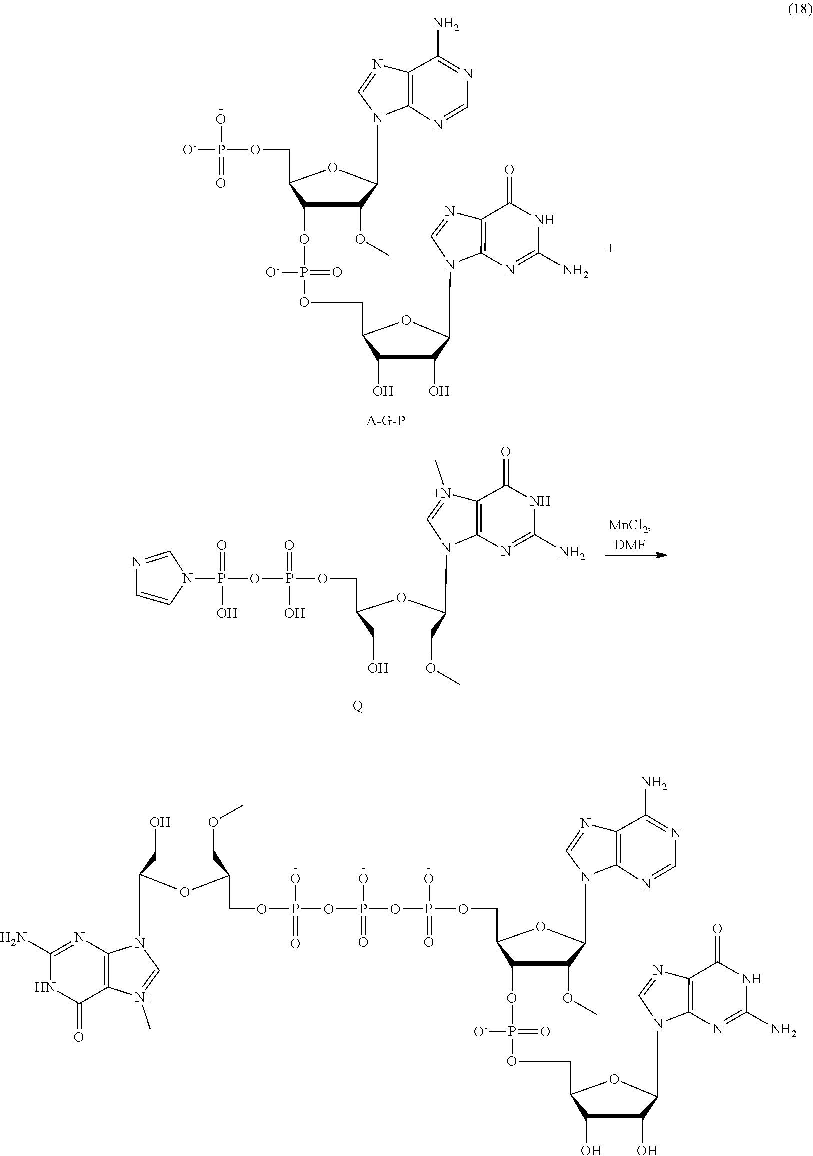

Wherein, compound Q was prepared through the following steps:

(1) 100 g of N²-Trt-5′-Trt-G was dissolved in methanol (30V) and water (10V), added with sodium periodate (1.1 eq), then stirred at room temperature for 16 h, concentrated under reduced pressure to remove most of the methanol, and added with ethyl acetate (10V). The organic phase was washed with water and concentrated under reduced pressure to obtain intermediate Q1, which required no further purification and can be directly added to the next step.

(2) Intermediate Q1 was dissolved in ethanol (20V), slowly added with sodium borohydride (3.0 eq) at room temperature and then stirred for 3 h. Acetone (30.0 eq) was carefully added dropwise to the reaction solution to quench excess sodium borohydride. After the addition was completed, the reaction solution was stirred for 1 h, then distilled under reduced pressure and then dissolved in dichloromethane (15V). The organic phase was washed with 3N ammonium chloride aqueous solution and water respectively, dried over anhydrous sodium sulfate and concentrated under reduced pressure to obtain the crude product. The crude product was recrystallized with ethyl acetate to obtain pure intermediate Q2.

(3) Intermediate Q2 was dissolved in anhydrous pyridine (10V), slowly added with p-toluenesulfonyl chloride (2.2 eq), and then stirred at room temperature for 3 h. The reaction mixture was concentrated under reduced pressure to remove pyridine and then purified by column chromatography to obtain intermediate Q3.

(4) Intermediate Q3 was dissolved in 1N sodium methoxide solution (30V) and heated to 60° C. for 48 h of reaction. After cooling to room temperature, an equal volume of dichloromethane was added, and the pH was adjusted to neutral with Dowex 50 (H⁺) resin. After suction filtration was performed, the filtrate was concentrated under reduced pressure and purified by column chromatography to obtain intermediate Q4.

(5) Intermediate Q4 was dissolved in 80% acetic acid solution (15V), heated to 60° C., and stirred for 5 h. The mixture was concentrated under reduced pressure to remove the solvent, and purified by reverse-phase preparative chromatography to obtain intermediate Q5.

Intermediates Q6, Q7, Q8, Q9, and Q were prepared respectively by referring to the synthetic methods of intermediates F, G, H, I, and J. The reaction route is shown in the following equation (19):

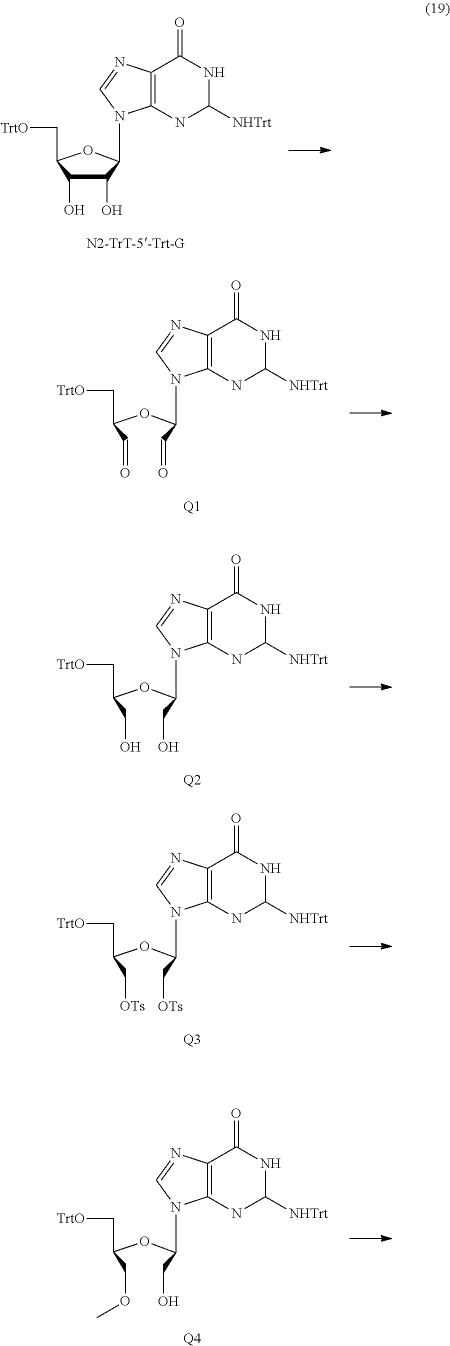

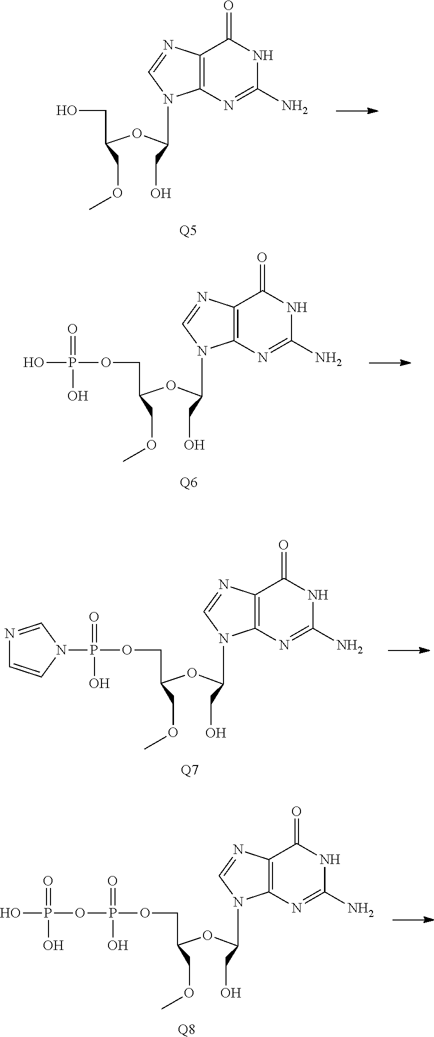

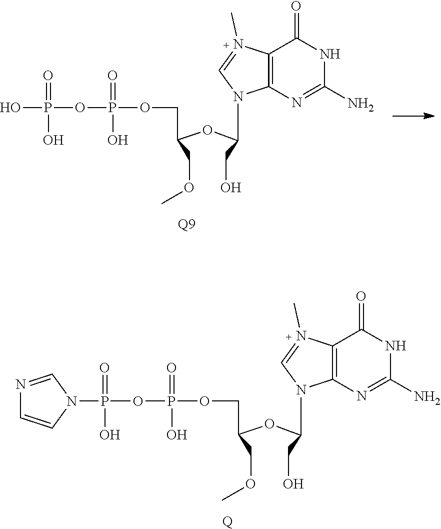

Example 12: Synthesis method of an oligonucleotide primer with an acyclic nucleoside structure for initial capping using intermediates A-G-P and R as raw materials

The oligonucleotide primer for initial capping in Example 12 was prepared using intermediates A-G-P and R as raw materials by referring to the synthesis method of the target product in Example 1. The reaction route is as follows:

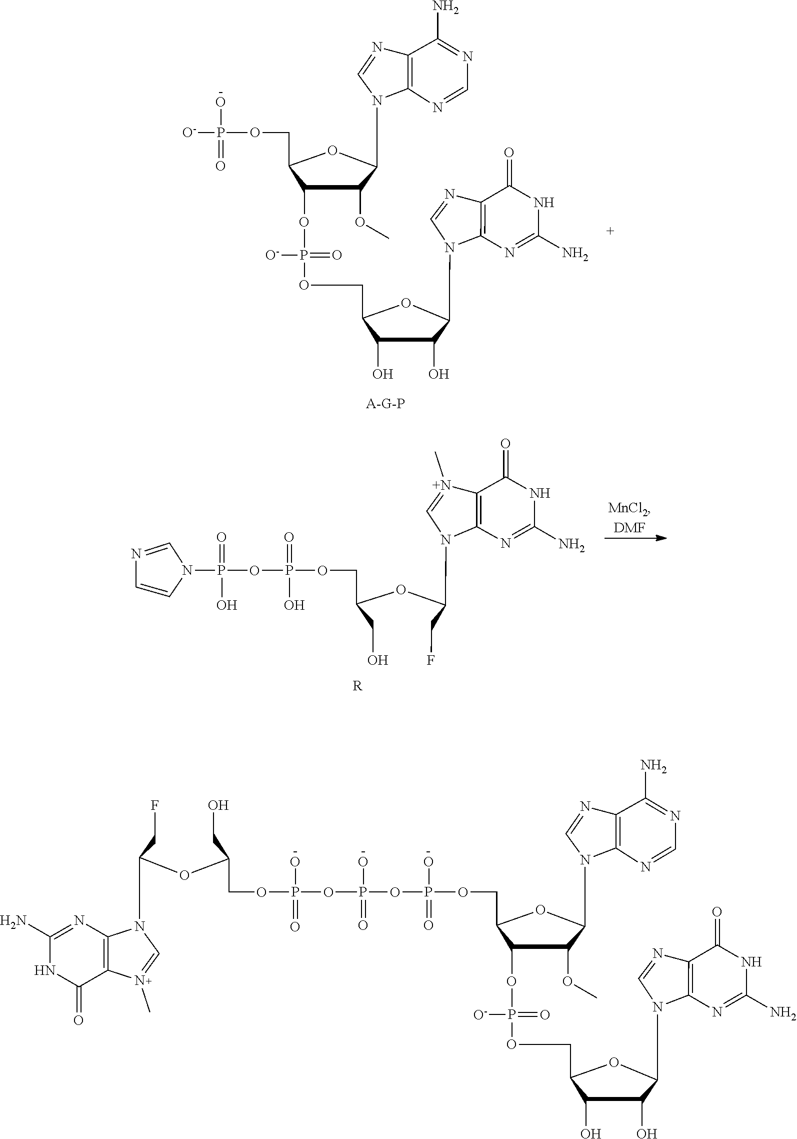

Wherein, compound R was prepared through the following steps:

(1) Intermediate Q2 (20 g) was dissolved in dichloromethane, and imidazole (1.5 eq) and tert-butyldimethylchlorosilane (1.3 eq) were added to the reaction solution. The reaction solution was stirred at room temperature for 2 h, washed with saturated sodium bicarbonate aqueous solution and saturated sodium chloride, dried over anhydrous sodium sulfate, concentrated under reduced pressure, and purified by column chromatography to obtain intermediate R1.

(2) Intermediate R1 was dissolved in anhydrous dichloromethane (10V), added with pyridine (1.5V), cooled to 0° C., slowly added with DAST, and stirred overnight after returning to room temperature. The pH was adjusted to neutral by slowly adding saturated sodium bicarbonate aqueous solution dropwise. After the reaction solution was washed with water, the organic phase was dried over anhydrous sodium sulfate, concentrated under reduced pressure, and purified by column chromatography to obtain intermediate R2.

Intermediates R3, R4, R5, R6, R7, and R were prepared respectively by referring to the synthetic methods of intermediates Q5, Q6, Q7, Q8, Q9, and Q. The reaction route is shown in the following equation (20):

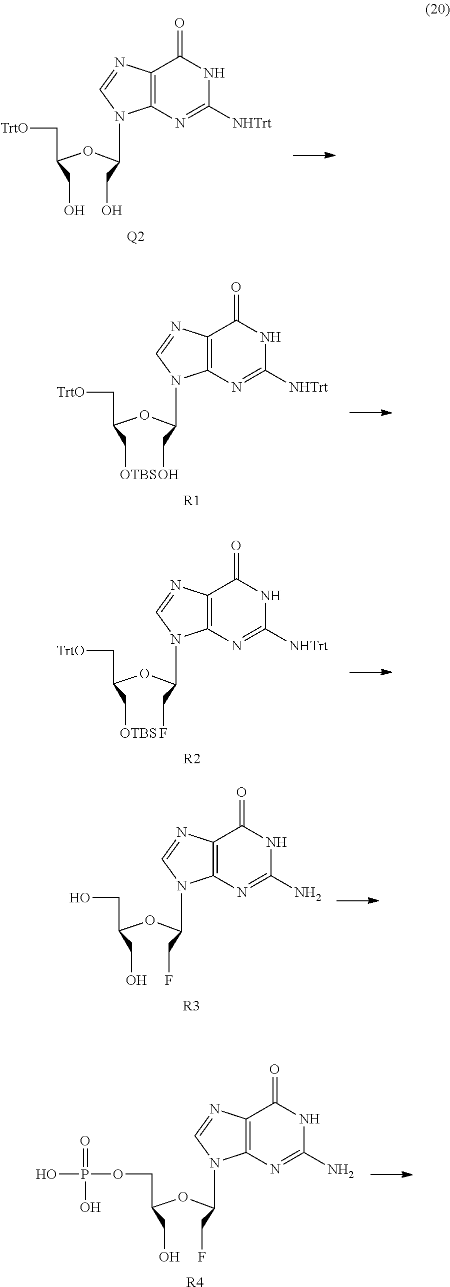

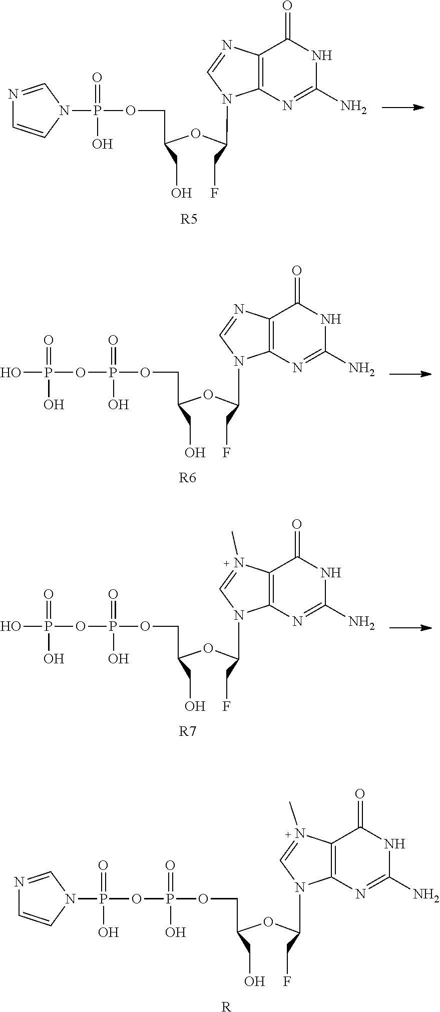

Example 13: Synthesis method of an oligonucleotide primer with an acyclic nucleoside structure for initial capping using intermediates A-G-P and S as raw materials

The oligonucleotide primer for initial capping in Example 13 was prepared using intermediates A-G-P and S as raw materials by referring to the synthesis method of the target product in Example 1. The reaction route is shown in the following equation (21):

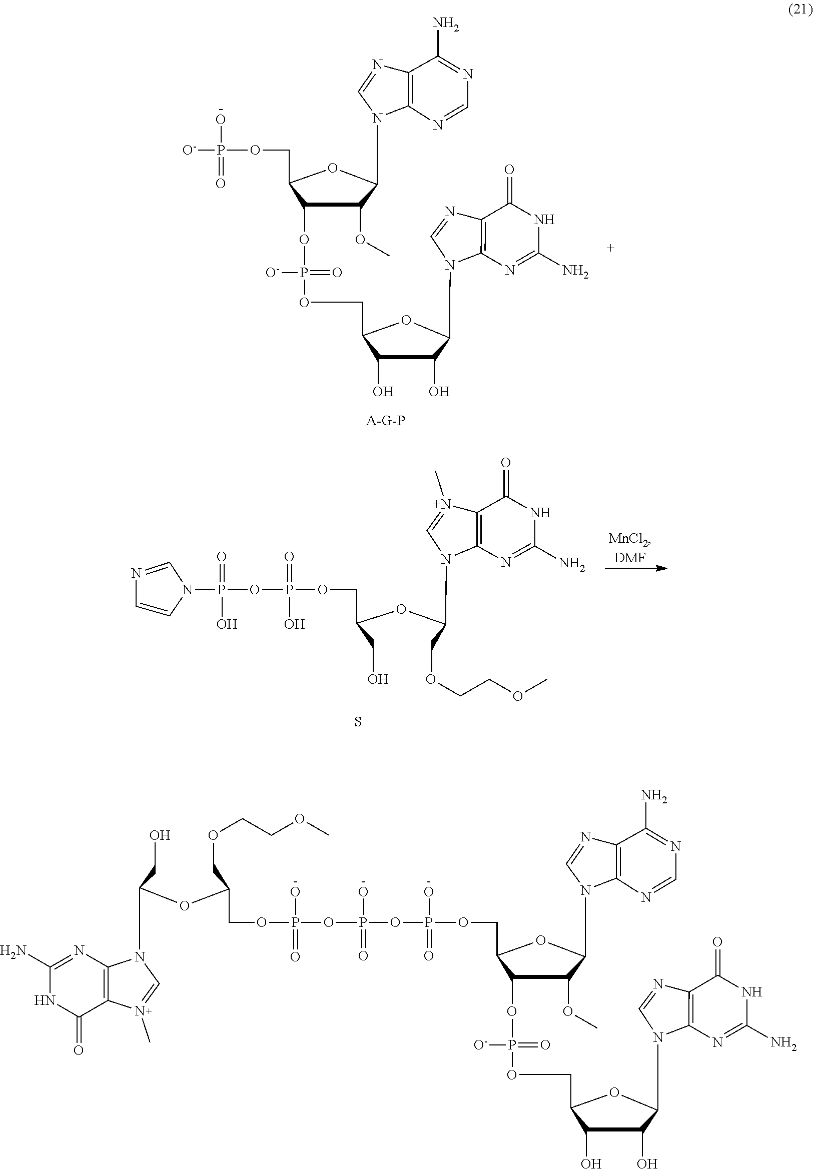

Wherein, compound S was prepared through the following steps:

A solution of intermediate R1 (50 g) in DMF (6V) was added dropwise to a suspension of sodium hydride (60%, 1.5 eq) in DMF (6V) at 0° C. and stirred at 0° C. for 0.5 h. The reaction solution was added with a solution of 2-bromoethyl methyl ether (1.5 eq) in DMF (2V) dropwise at 0° C., and stirred for 4 h after warming to room temperature. The reaction solution was quenched with saturated ammonium chloride aqueous solution, extracted with ethyl acetate, concentrated under reduced pressure, and purified by column chromatography to obtain intermediate S1.

Intermediates S2, S3, S4, S5, S6, and S were prepared respectively by referring to the synthetic methods of intermediates R3, R4, R5, R6, R7, and R. The reaction route is shown in the following equation (22):

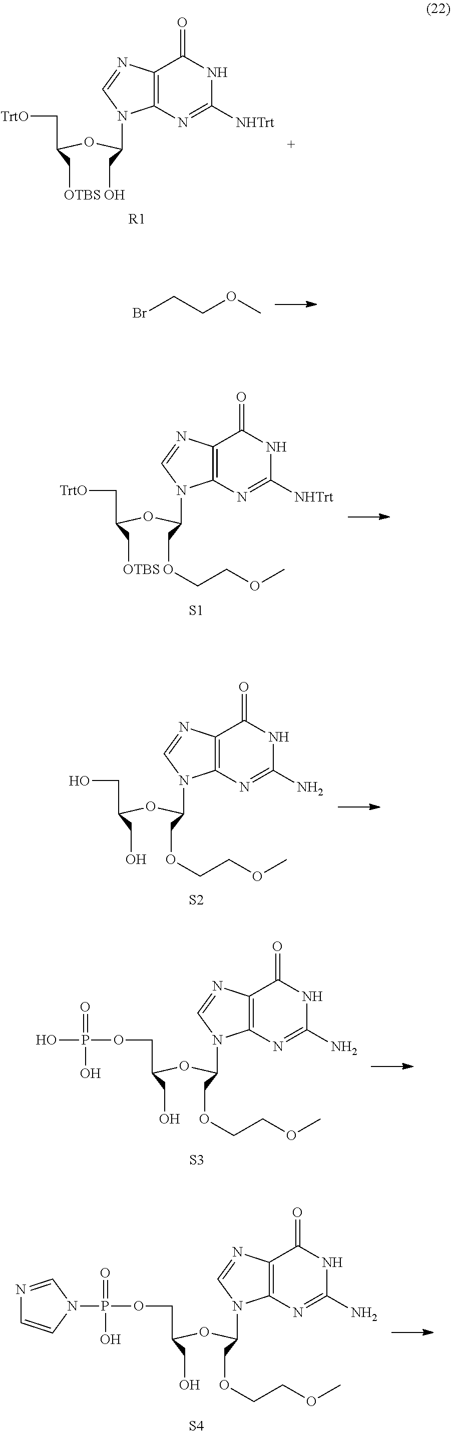

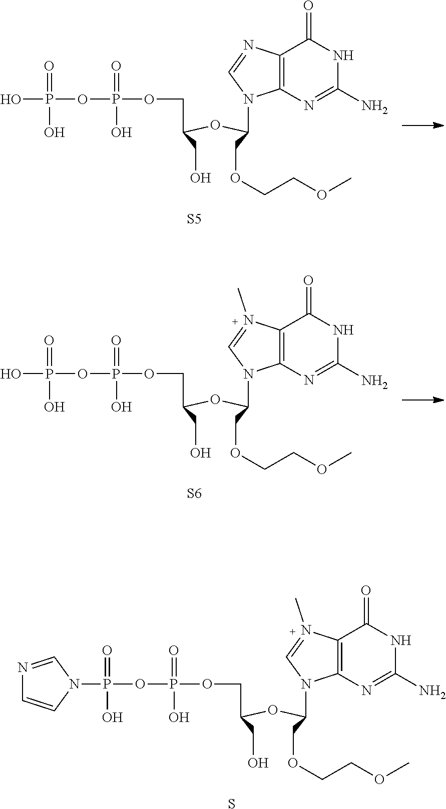

Example 14: Synthesis method of an oligonucleotide primer with an acyclic nucleoside structure for initial capping using intermediates UrA-G-P and T as raw materials

The oligonucleotide primer for initial capping in Example 14 was prepared using intermediates UrA-G-P and T as raw materials by referring to the synthesis method of the target product in Example 1. The reaction route is shown in the following equation (23):

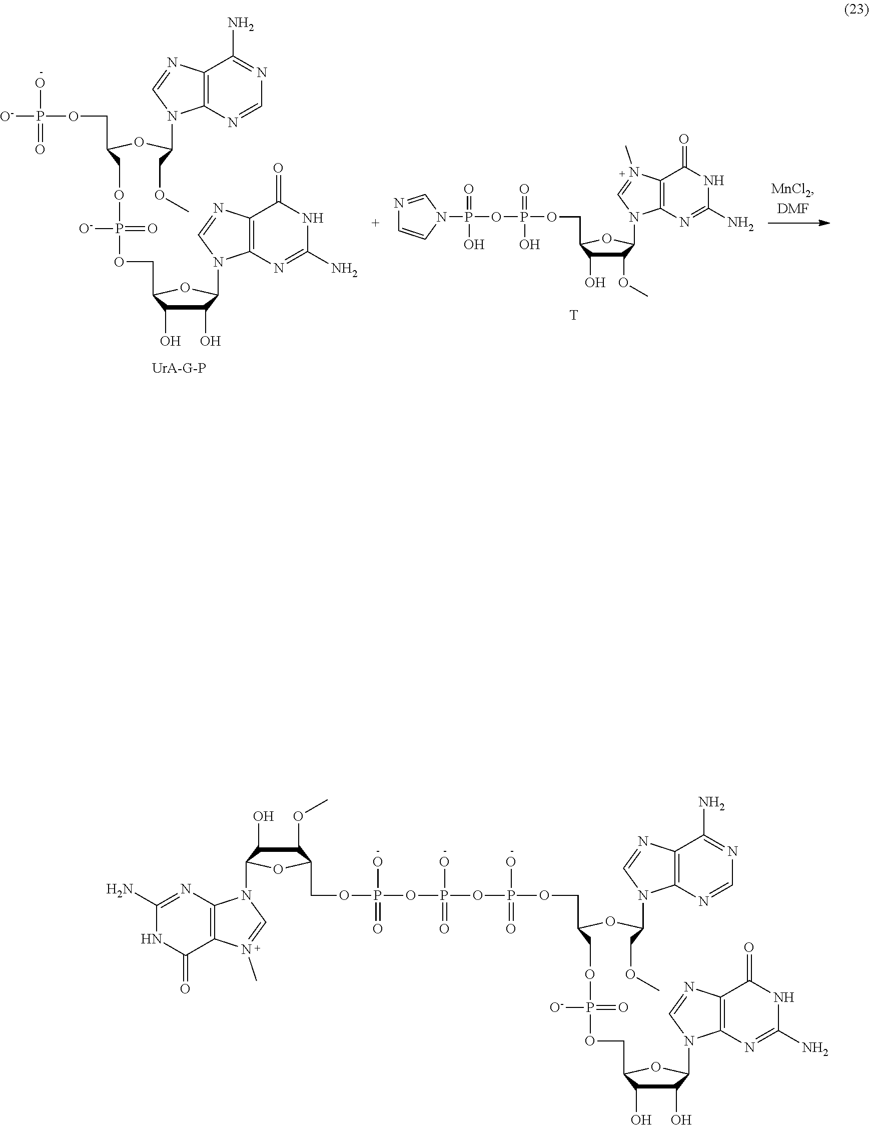

Wherein, compound T was prepared through the following steps:

Intermediates T1 to T were prepared respectively by referring to the synthesis methods of intermediates Q6 to Q in Example 11. The reaction route is shown in the following equation (24):

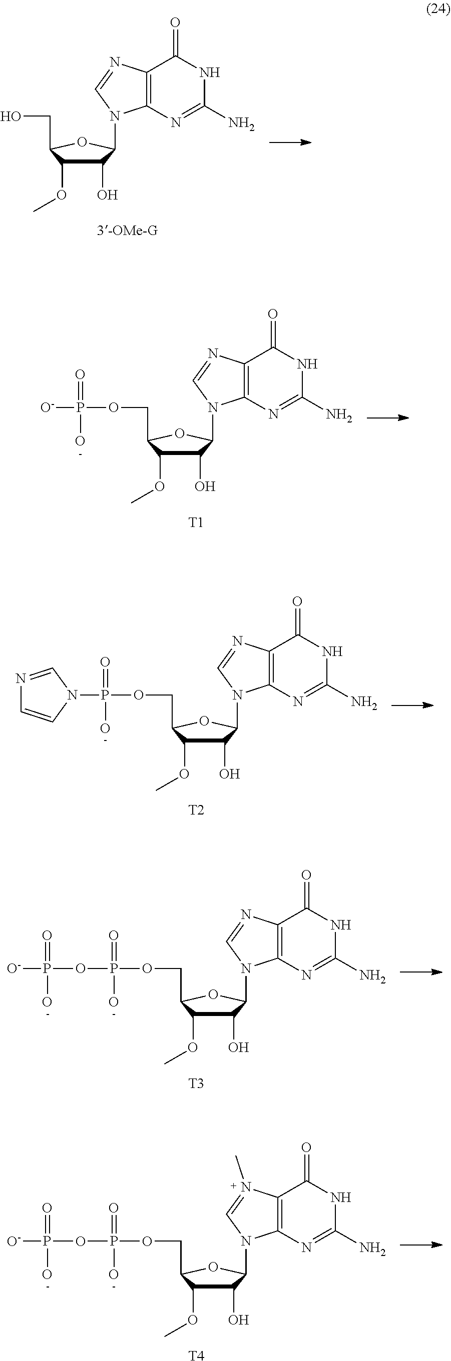

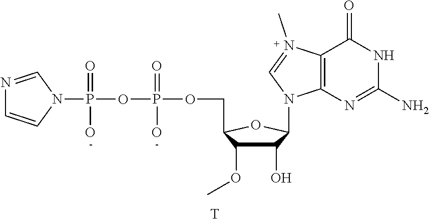

Example 15: Synthesis method of an oligonucleotide primer with an acyclic nucleoside structure for initial capping using intermediates UrA-G-P and U as raw materials

The oligonucleotide primer for initial capping in Example 15 was prepared using intermediates UrA-G-P and U as raw materials by referring to the synthesis method of the target product in Example 1. The reaction route is shown in the following equation (25):

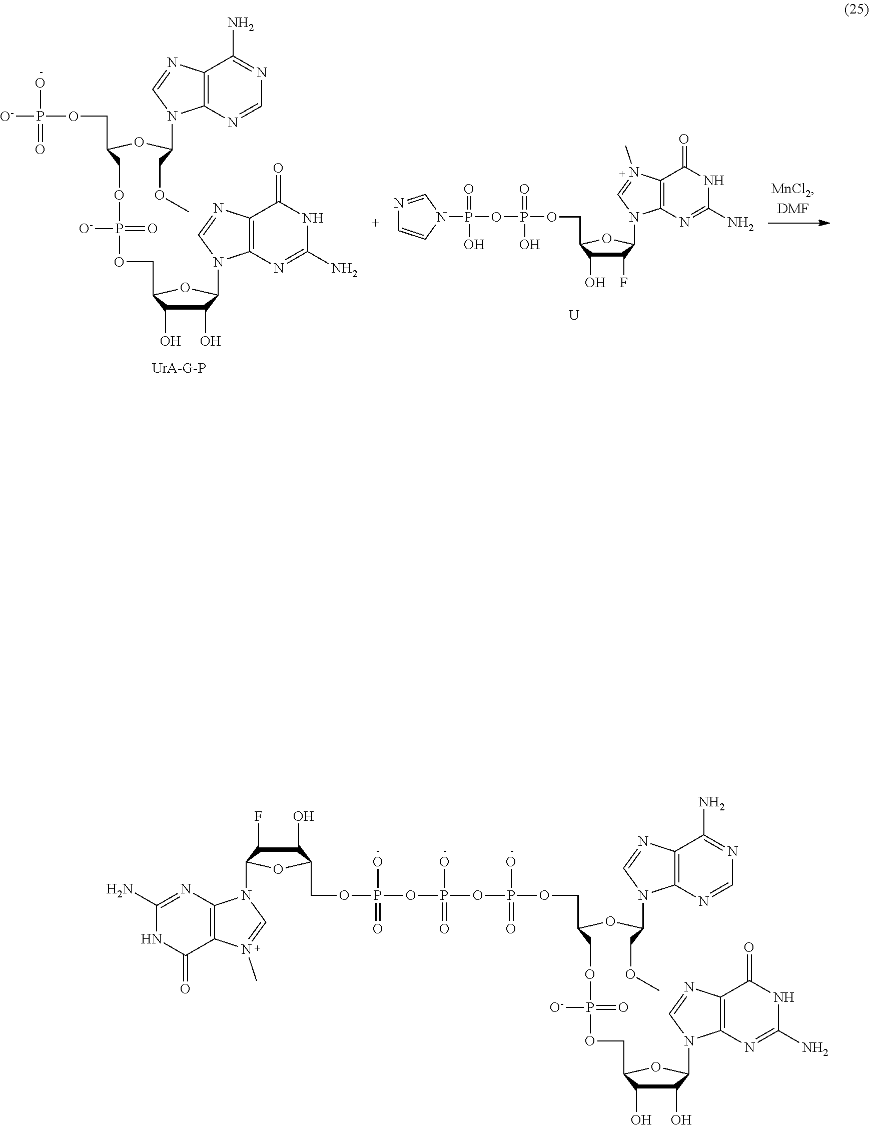

Wherein, compound U was prepared through the following steps: Intermediates U1 to U were prepared respectively by referring to the synthesis methods of intermediates T1 to T in Example 14. The reaction route is shown in the following equation (26):

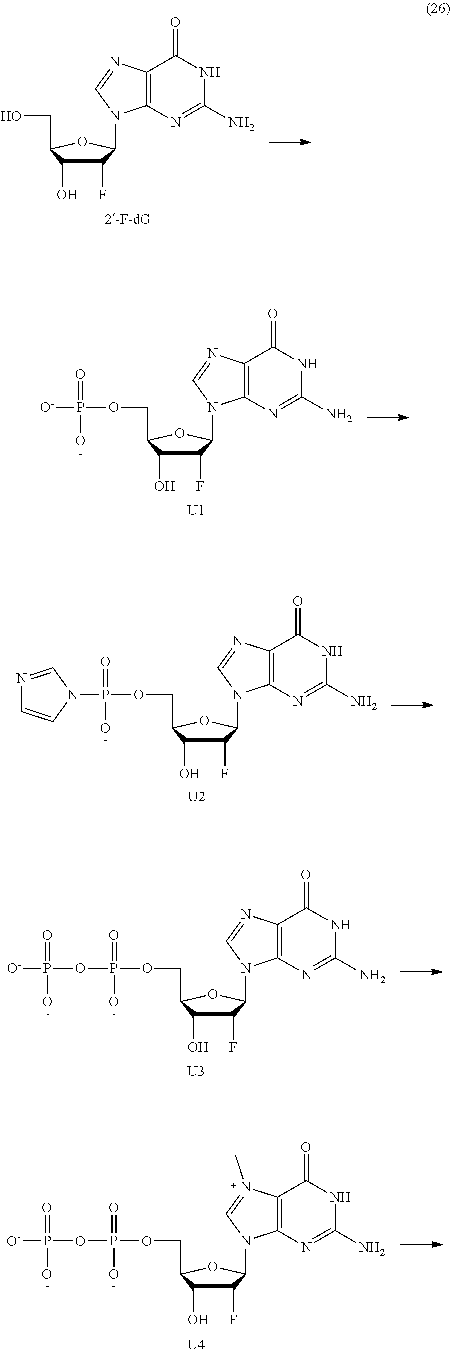

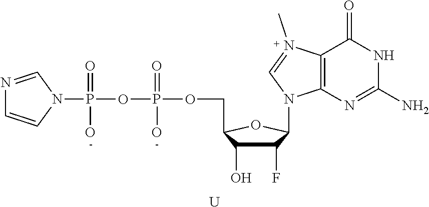

Example 16: Synthesis method of an oligonucleotide primer with an acyclic nucleoside structure for initial capping using intermediates UrA-G-P and V as raw materials

The oligonucleotide primer for initial capping in Example 16 was prepared using intermediates UrA-G-P and V as raw materials by referring to the synthesis method of the target product in Example 1. The reaction route is shown in the following equation (27):

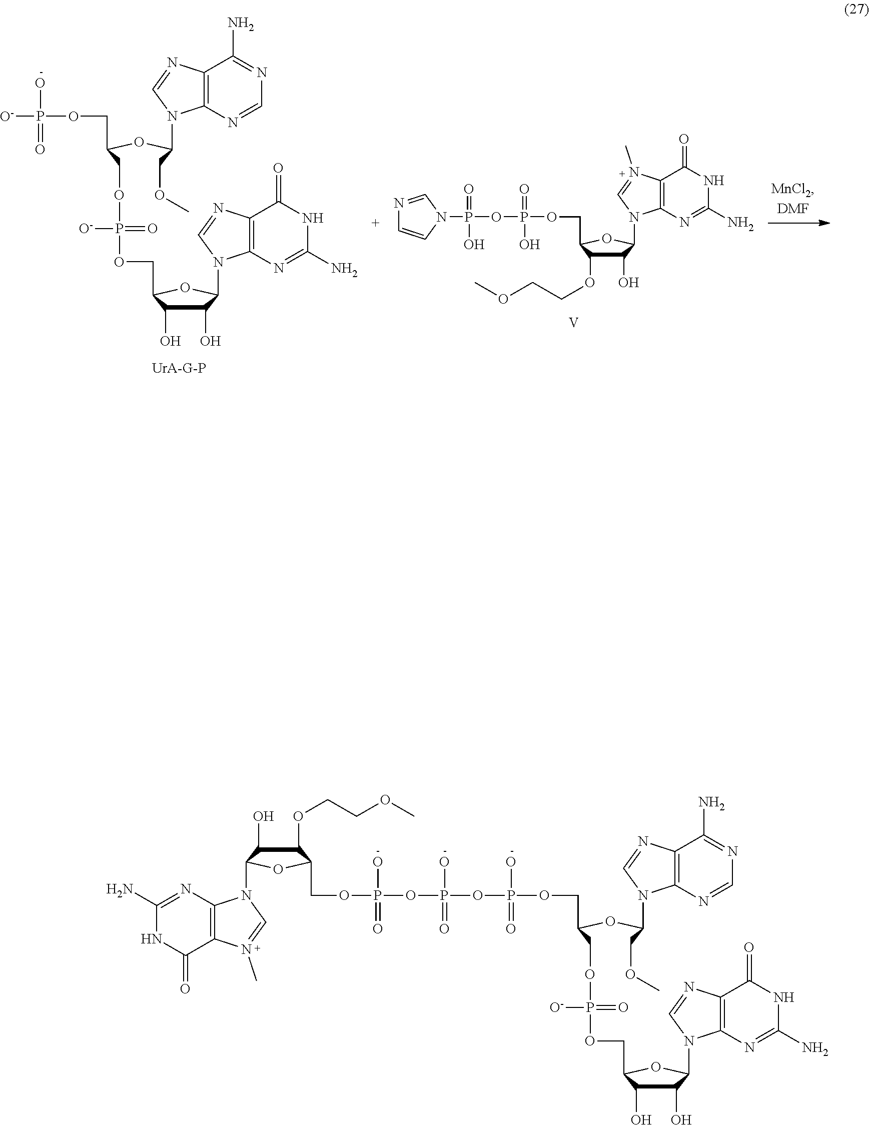

Wherein, compound V was prepared through the following steps: Intermediates V1 to V were prepared respectively by referring to the synthesis methods of intermediates T1 to T in Example 14. The reaction route is shown in the following equation (28):

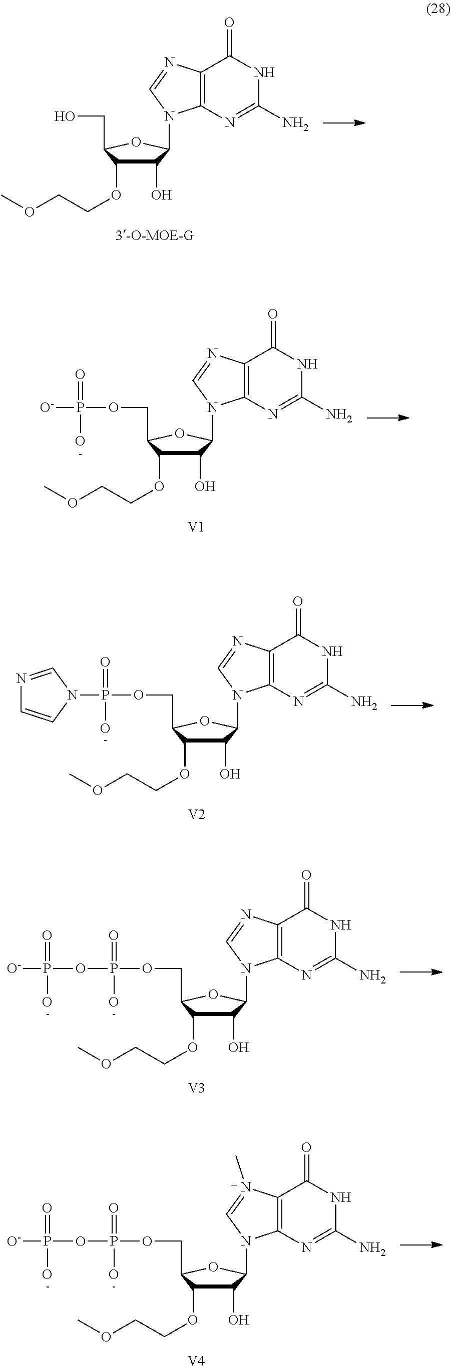

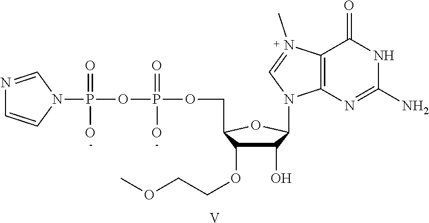

Example 17: Synthesis method of an oligonucleotide primer with an acyclic nucleoside structure for initial capping using intermediates UrA-G-P and W as raw materials

The oligonucleotide primer for initial capping in Example 17 was prepared using intermediates UrA-G-P and W as raw materials by referring to the synthesis method of the target product in Example 1. The reaction route is shown in the following equation (29):

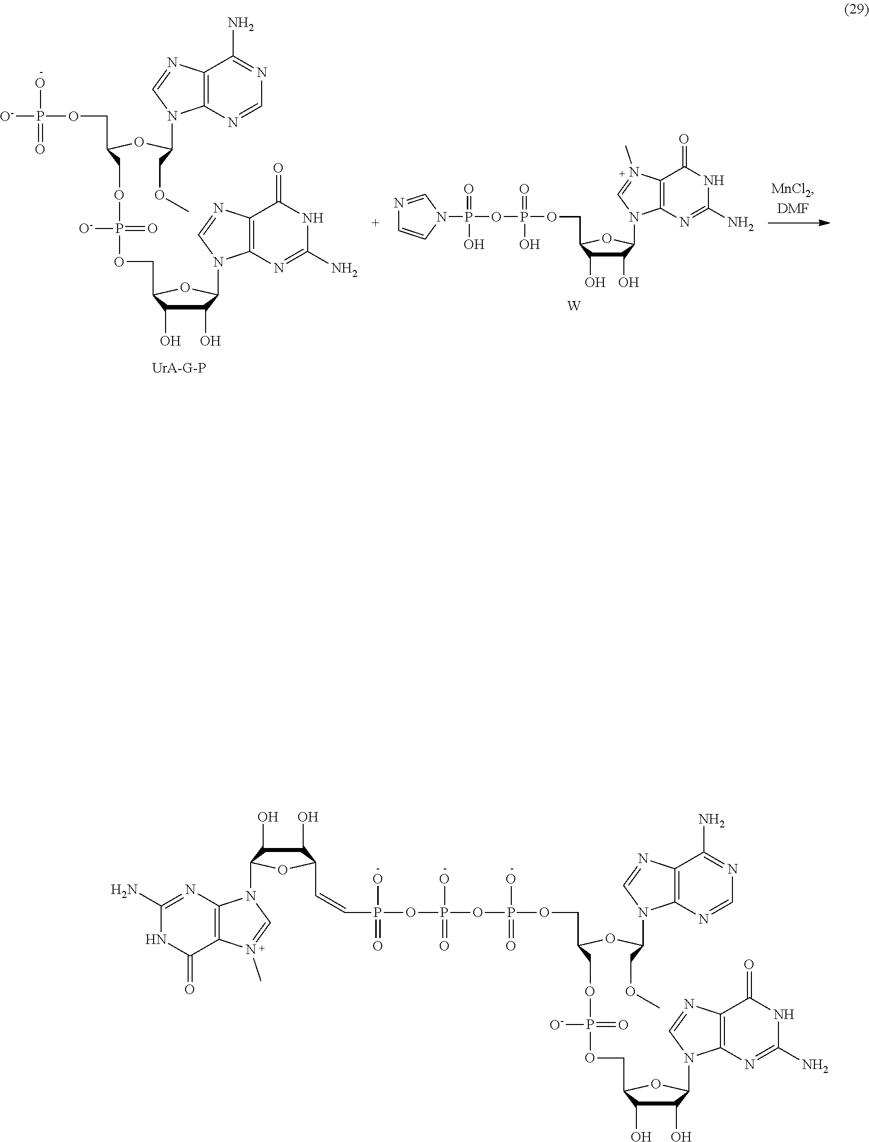

Wherein, compound W was prepared through the following steps: Intermediates W2 to W were prepared respectively by referring to the synthesis methods of intermediates V2 to V in Example 16. The reaction route is shown in the following equation (30):

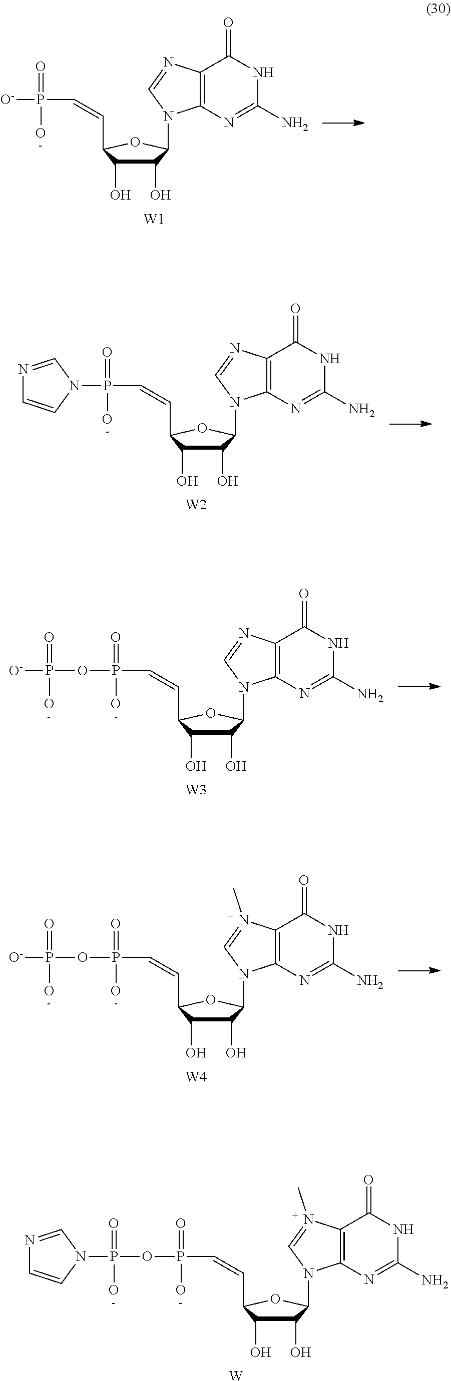

Example 18: Synthesis method of an oligonucleotide primer with an acyclic nucleoside structure for initial capping using intermediates UrA-G-P and X as raw materials

The oligonucleotide primer for initial capping in Example 18 was prepared using intermediates UrA-G-P and X as raw materials by referring to the synthesis method of the target product in Example 1. The reaction route is shown in the following equation (31):

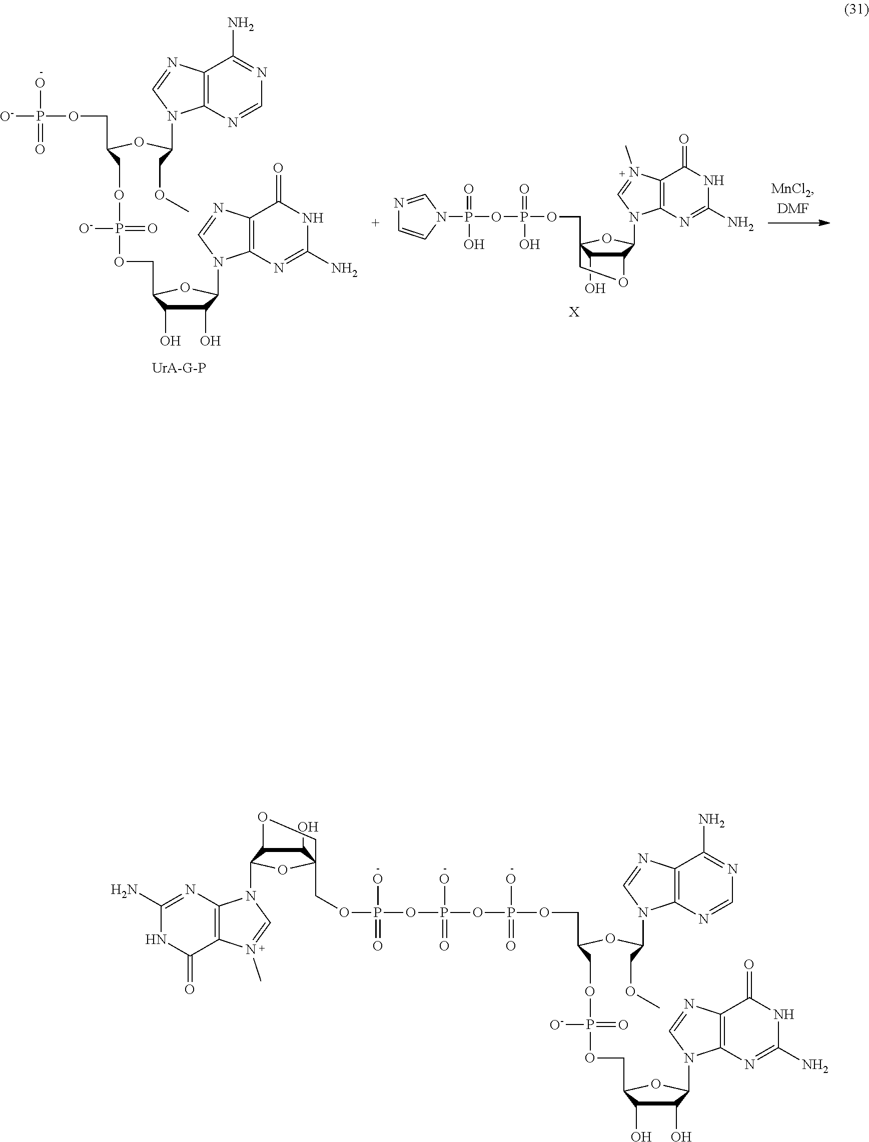

Wherein, compound X was prepared through the following steps: Intermediates X1 to X were prepared respectively by referring to the synthesis methods of intermediates V1 to V in Example 16. The reaction route is shown in the following equation (32):

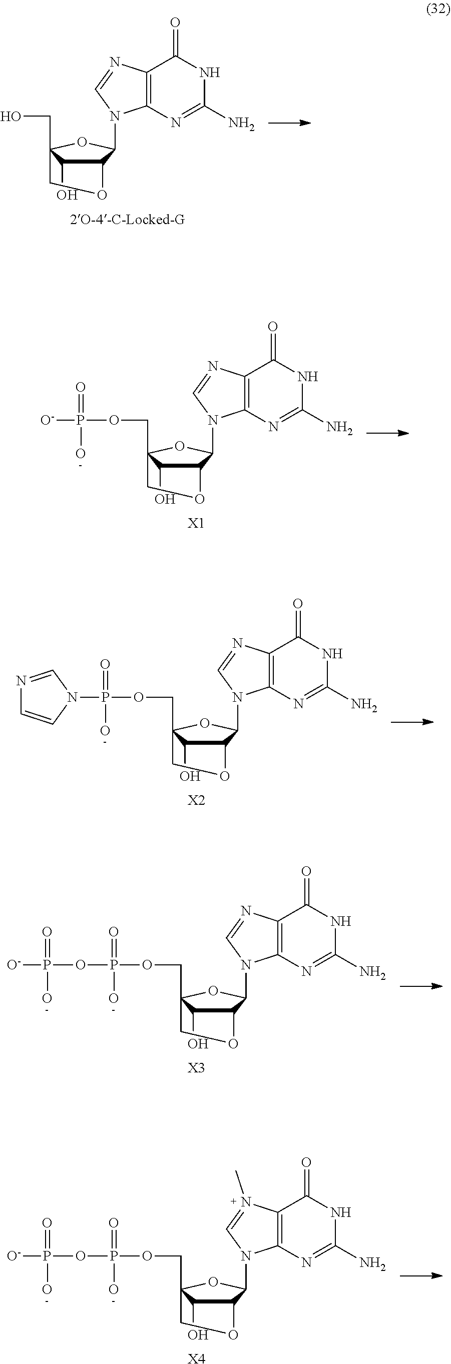

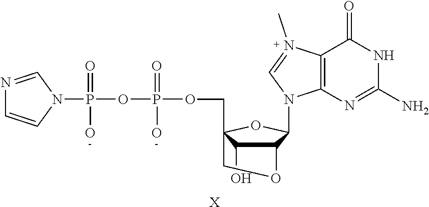

Example 19: Synthesis method of an oligonucleotide primer with an acyclic nucleoside structure for initial capping using intermediates UrA-G-P and Y as raw materials

The oligonucleotide primer for initial capping in Example 19 was prepared using intermediates UrA-G-P and Y as raw materials by referring to the synthesis method of the target product in Example 1. The reaction route is shown in the following equation (33):

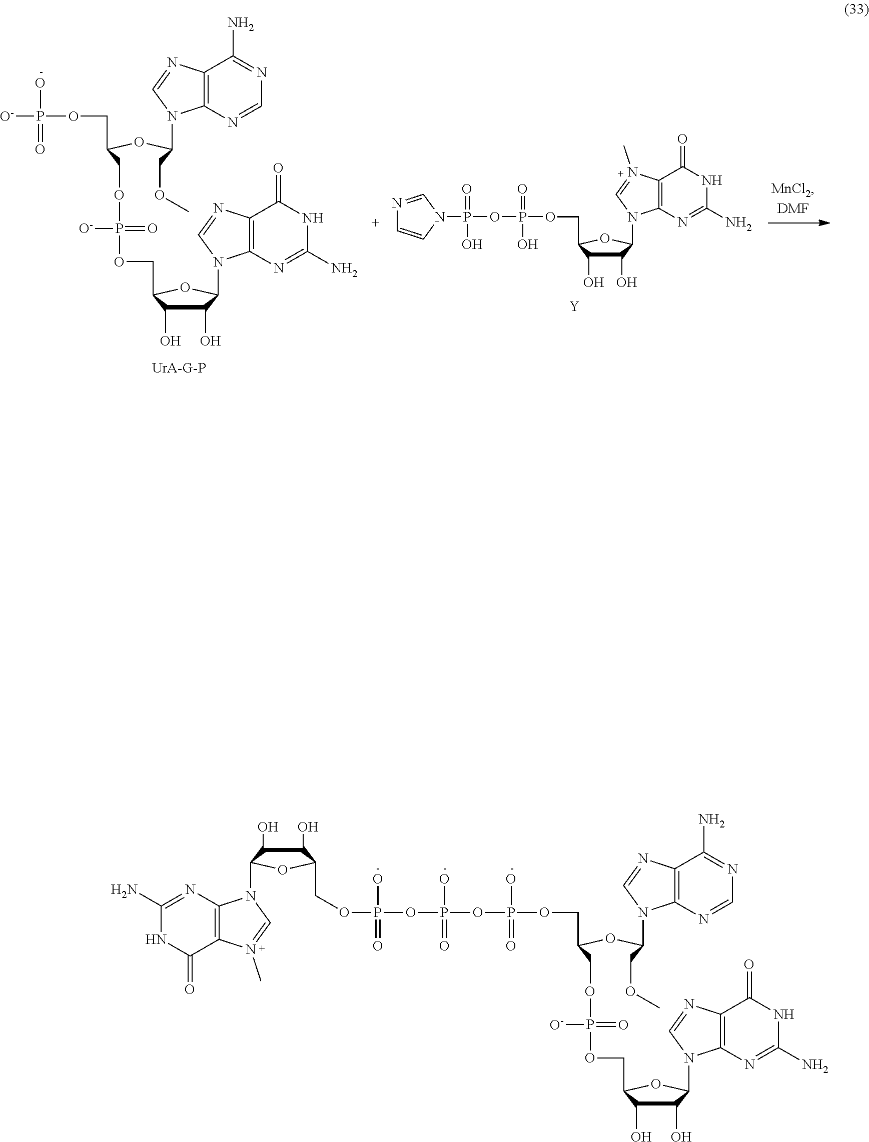

Wherein, compound Y was prepared through the following steps: Intermediates Y1 to Y were prepared respectively by referring to the synthesis methods of intermediates X3 to X in Example 16. The reaction route is shown in the following equation (34):

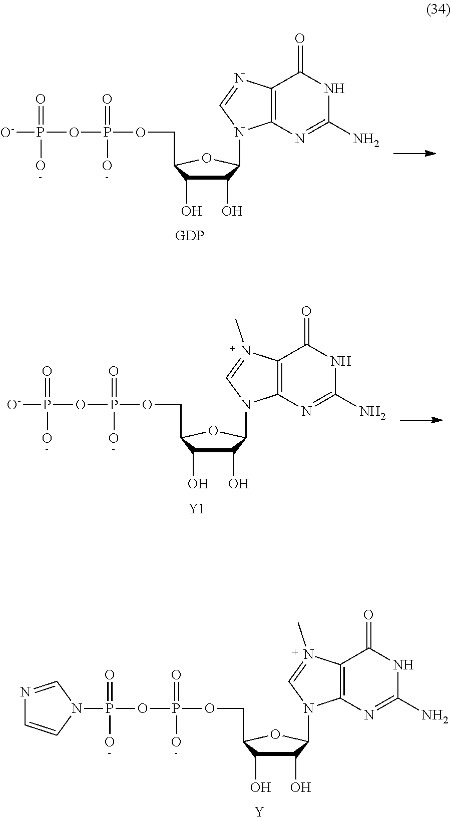

Comparative Example 1: ^{m7}GpppA_{2′OMe}pG

The synthesis method of ^{m7}GpppA_{2′OMe}pG (CleanCap® Reagent AG (Cat. No.: N-7113)) refers to the synthesis method of the above examples. The reaction route is shown in the following equation (35):

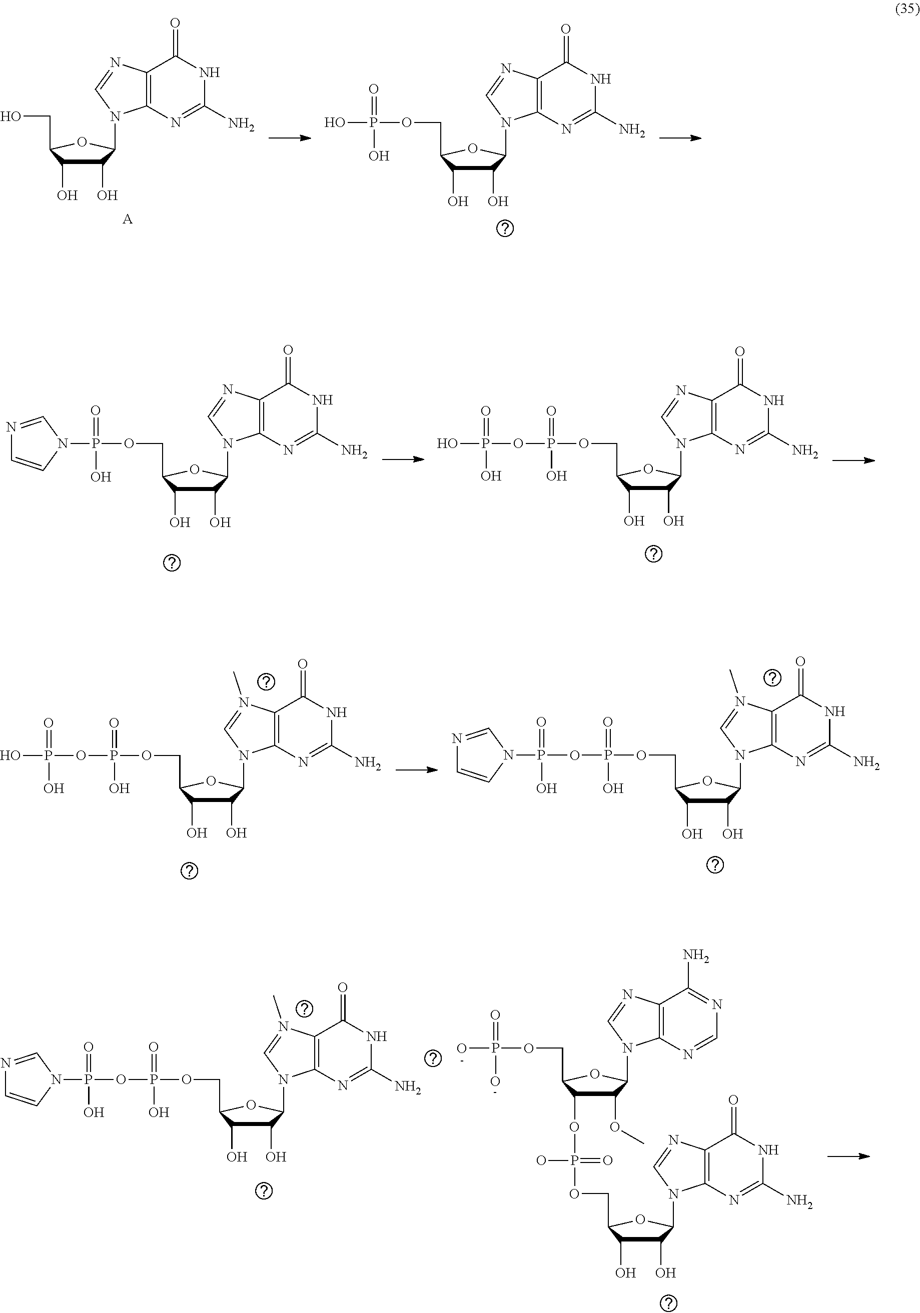

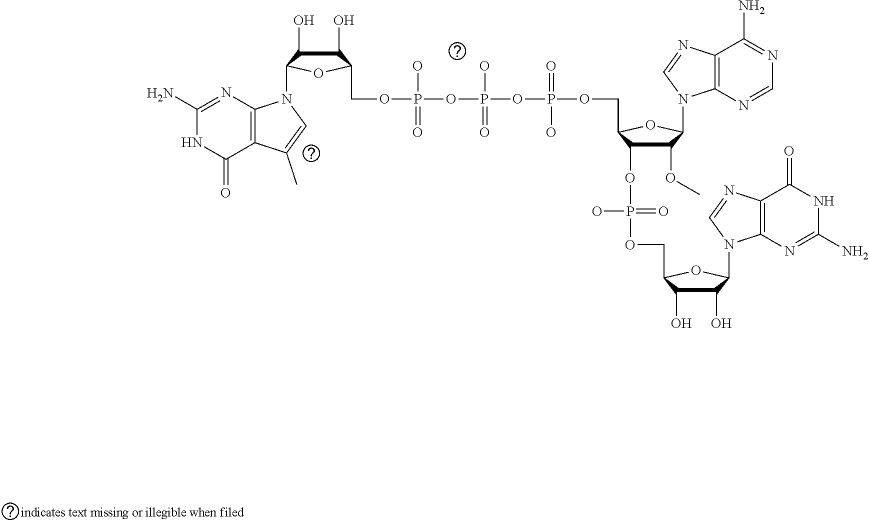

Comparative Example 2: The synthesis method of a capping analog in which only Rb has an acyclic structure refers to the synthesis method of the above examples. The reaction route is shown in the following equation (36):

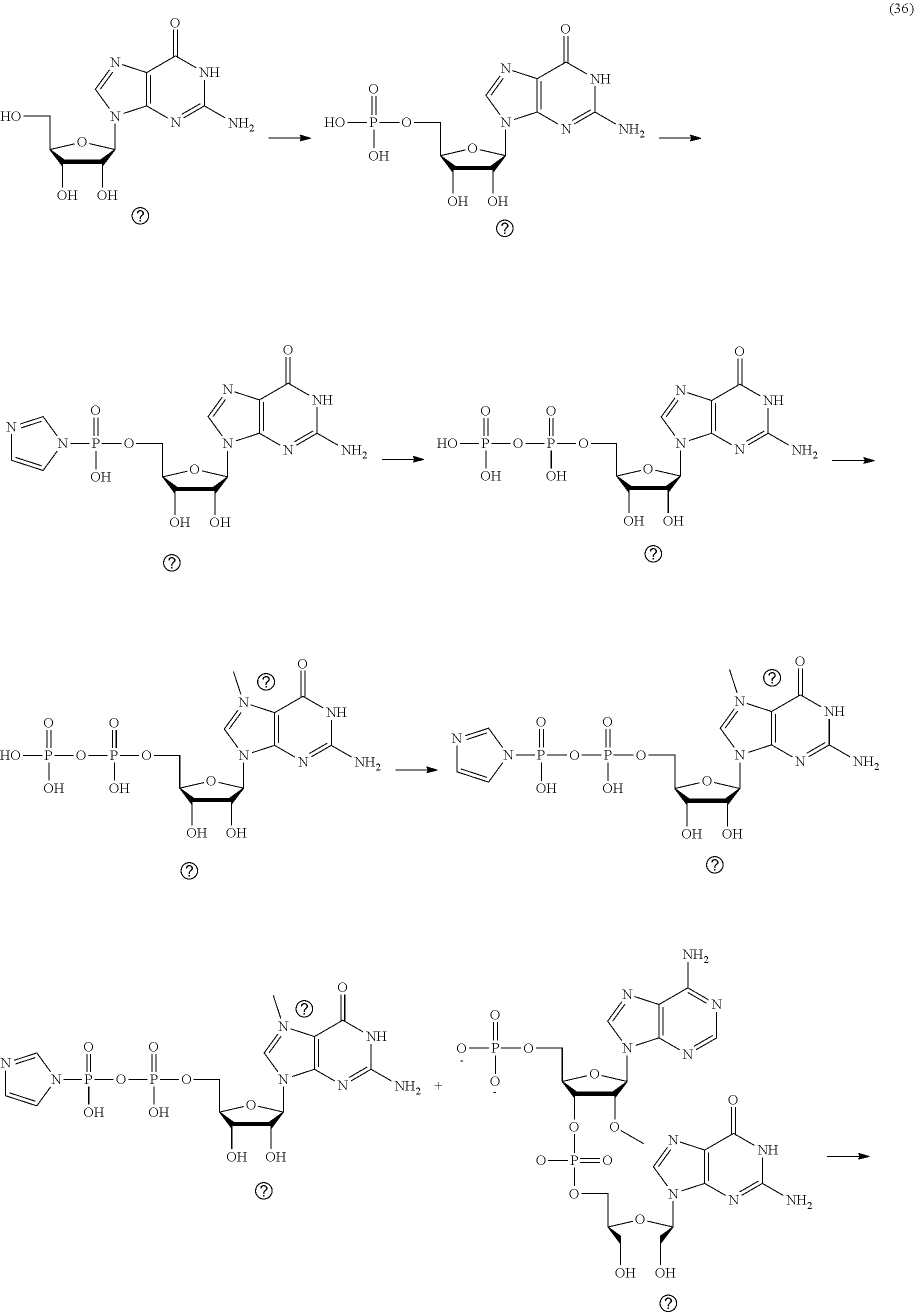

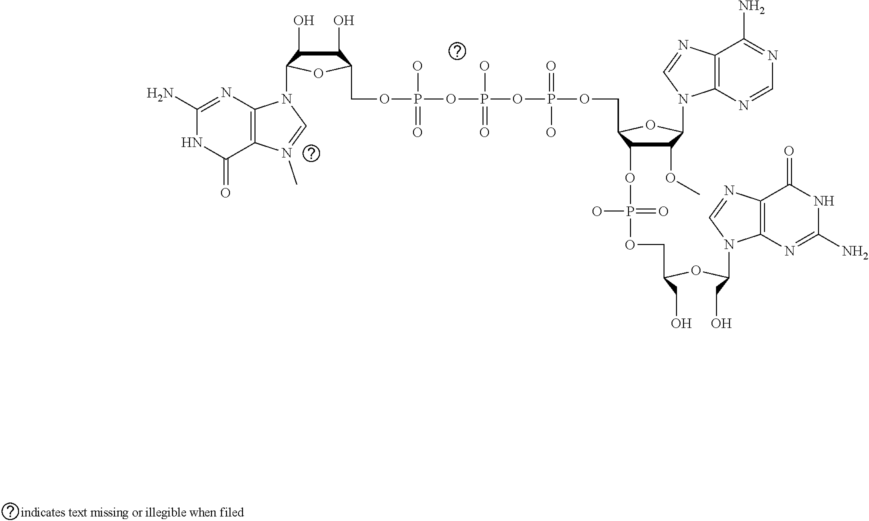

The structures of the oligonucleotide primers with an acyclic nucleoside structure for initial capping obtained in the examples and the capping analogs obtained in the comparative examples are shown in Table 2 below.

TABLE 2

| Number | Structure of capping analog |
|---|---|
| Example 1 | 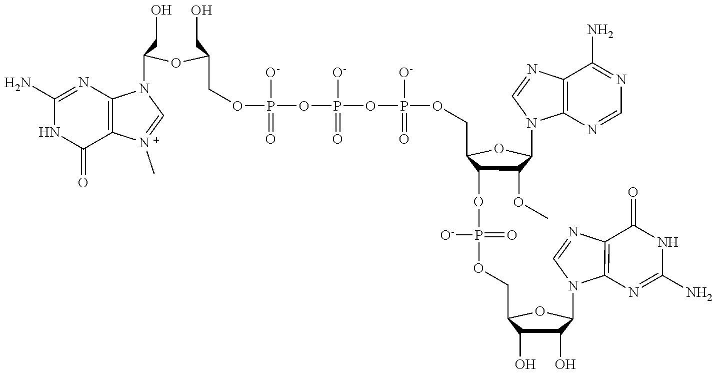 |
| Example 2 | 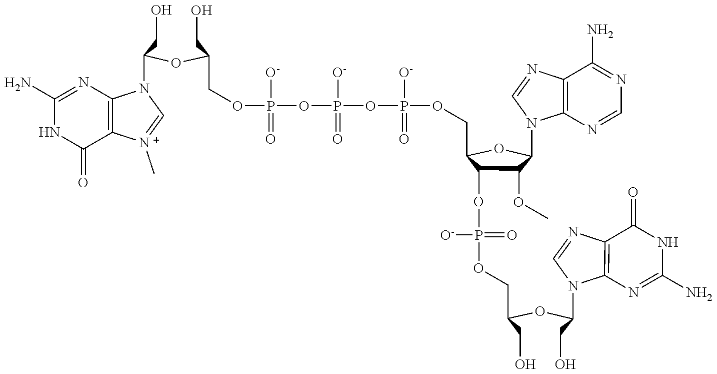 |
| Example 3 | 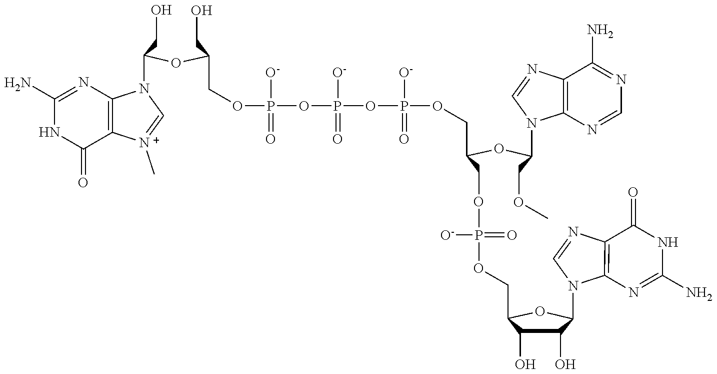 |
| Example 4 | 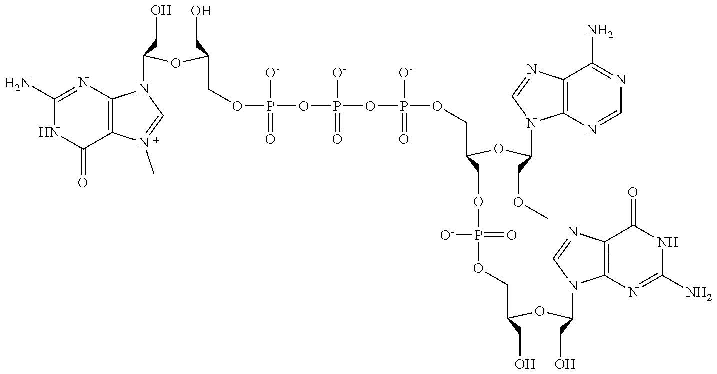 [SMILES: COC[C@@H](O[C@@H](COP(=O)([O-])OC[C@H](CO)O[C@H](CO)n1cnc2c(=O)[nH]c(N)nc21)COP(=O)([O-])OP(=O)([O-])OP(=O)([O-])OC[C@H](CO)O[C@H](CO)n1c[n+](C)c2c(=O)[nH]c(N)nc21)n1cnc2c(N)ncnc21] |
| Example 5 | 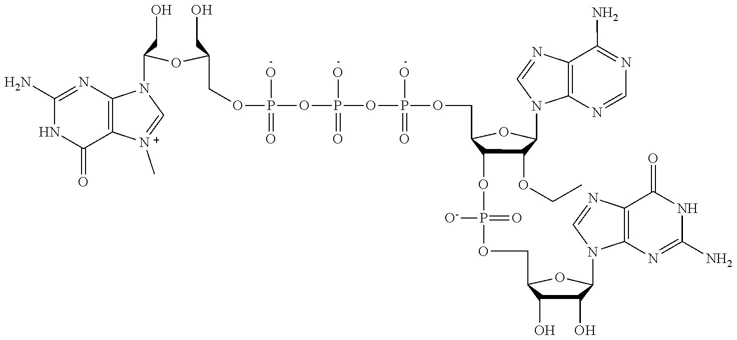 |
| Example 6 | 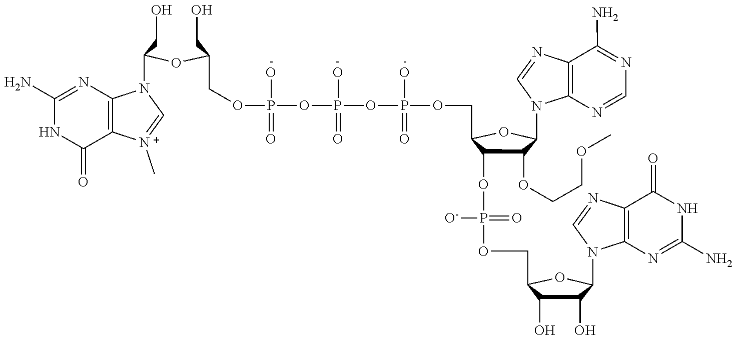 |
| Example 7 | 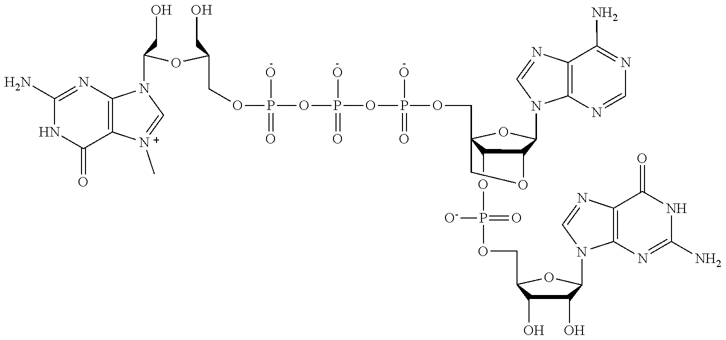 |
| Example 8 | 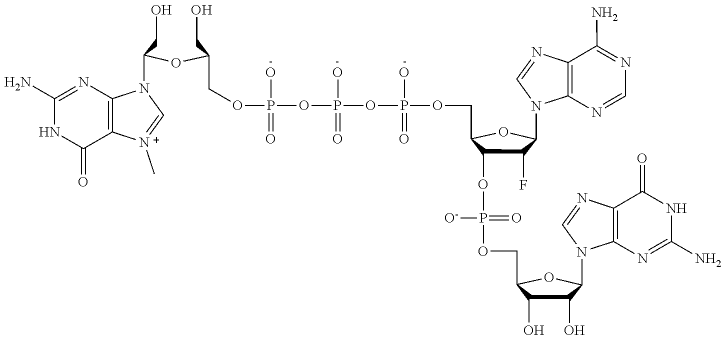 |
| Example 9 | 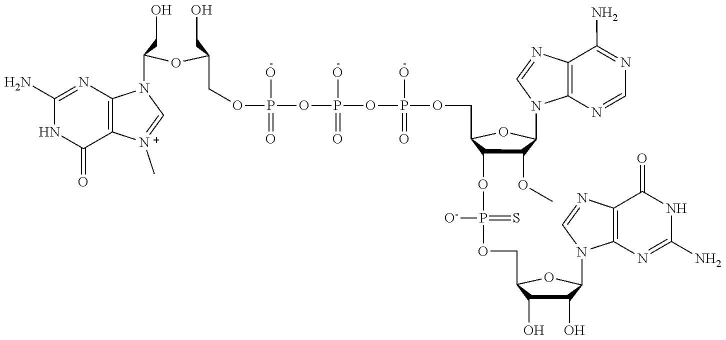 |
| Example 10 | 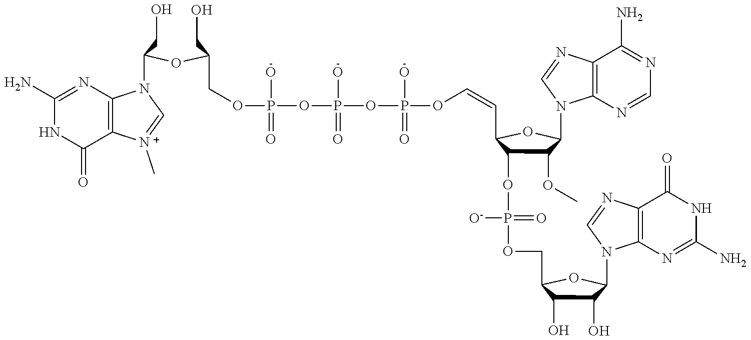 |
| Example 11 | 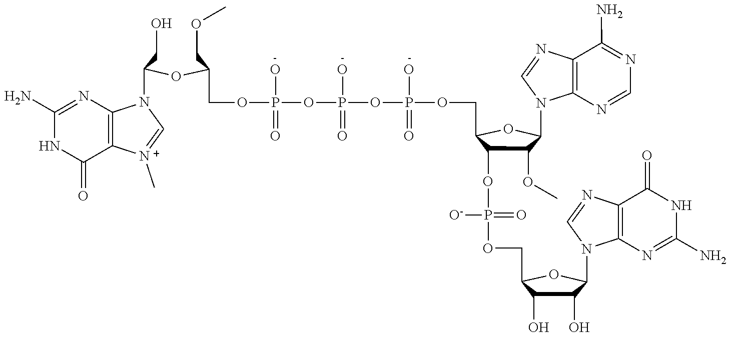 |
| Example 12 | 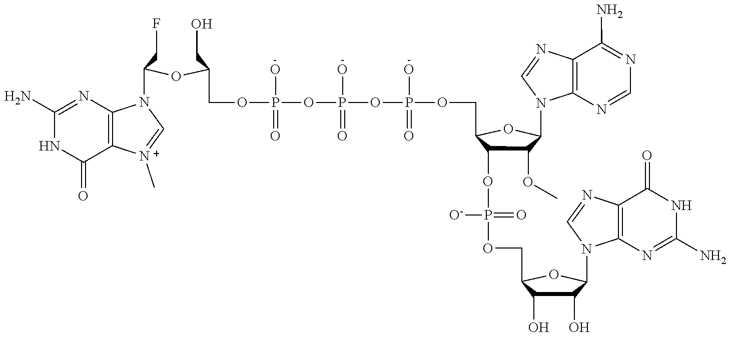 |
| Example 13 | 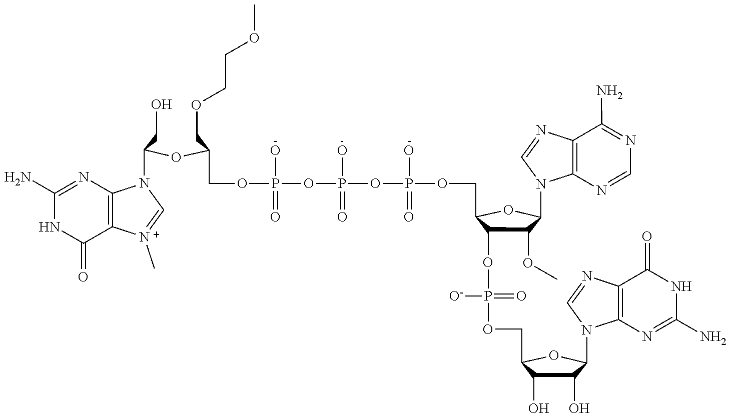 |
| Example 14 | 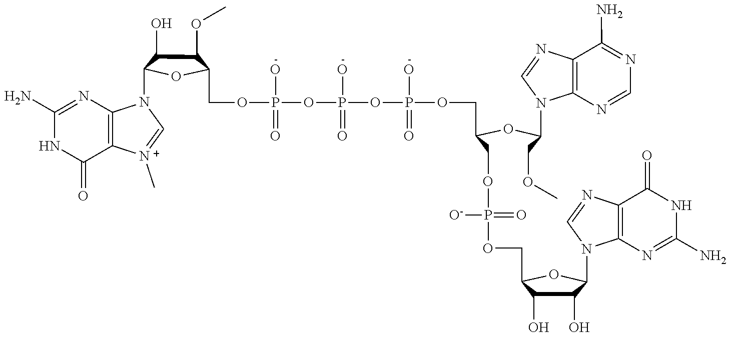 |
| Example 15 | 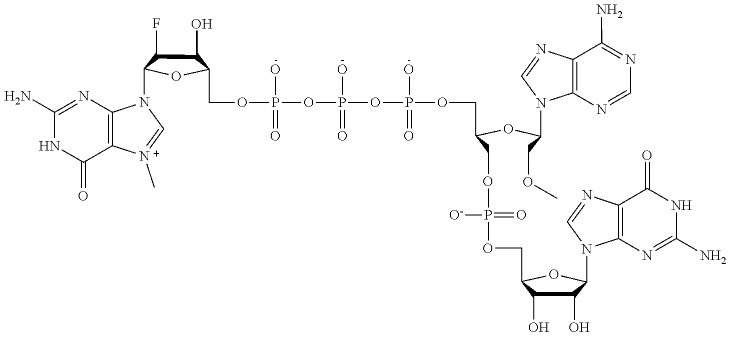 |
| Example 16 | 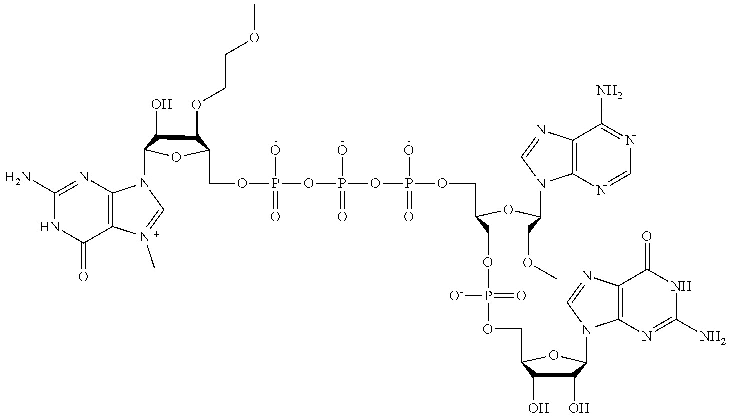 |
| Example 17 | 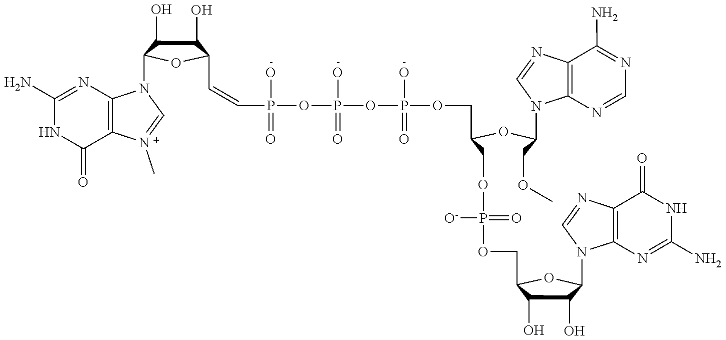 |
| Example 18 | 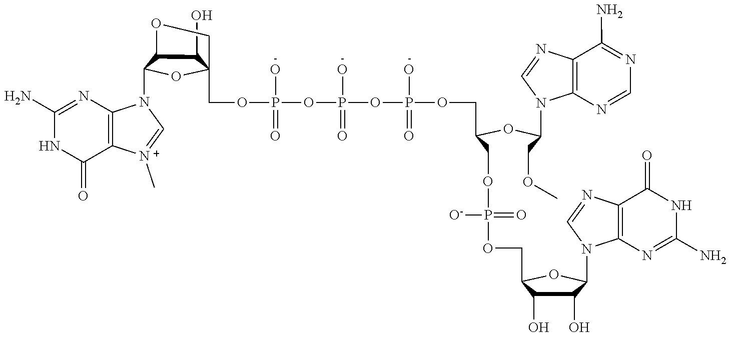 |
| Example 19 | 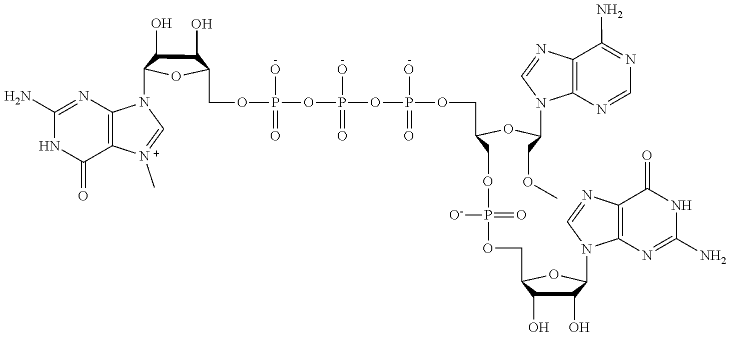 |
| Comparative Example 1 | 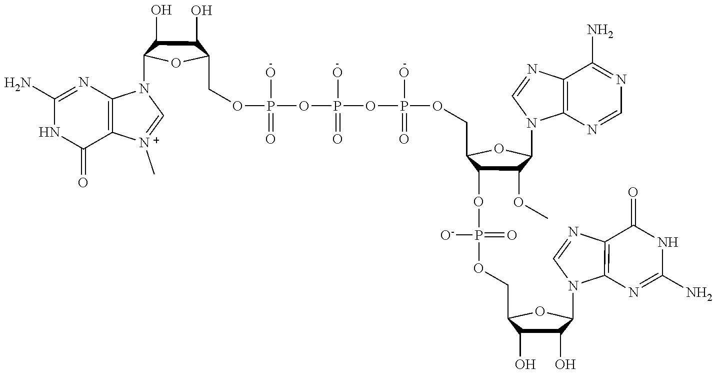 |
| Comparative Example 2 | 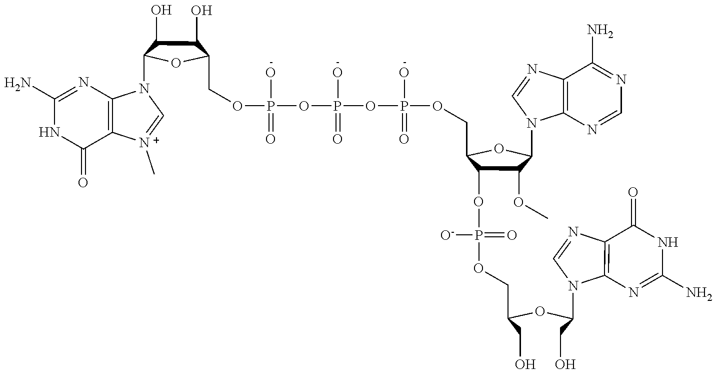 |

Test Example 1: Determination of in vitro transcription yield and capping efficiency of mRNA

In vitro synthesis of mRNA was performed using the oligonucleotide primer with an acyclic nucleoside structure for initial capping by the co-transcriptional capping method. First, the plasmid was digested with NotI overnight at 4° C. DNA template was extracted to synthesize mRNA by in vitro transcription. The oligonucleotide primers with an acyclic nucleoside structure for initial capping in Examples 1-4, 6, 7, and 11 and the capping analogs in Comparative Examples 1-2 were used as cap structures respectively.

The reaction system is shown in Table 3:

TABLE 3

| System | Amount | |
|---|---|---|
| T7 RNA polymerase | 50 | U |
| 10X buffer | 2 | μl |
| 100 mM ATP | 1 | μl |
| 100 mM GTP | 1 | μl |
| 100 mM CTP | 1 | μl |
| 100 mM N1-Me-pUTP | 1 | μl |
| 100 mM cap analog | 1 | μl |
| Inorganic pyrophosphatase | 0.05 | U |
| Nuclease inhibitor | 20 | U |
| Sterile enzyme-free water | Up to 20 μl | |
| Template | 1 | μg |

Note: During the experiment, the volume of materials required for the system was first calculated, and then the sample was added. The system was firstly added with sterile enzyme-free water, then added with 10X buffer, NTPs, and cap structure subsequently, mixed, centrifuged gently, then added with nuclease inhibitor, inorganic pyrophosphatase, T7 RNA polymerase, and linearized DNA template, mixed thoroughly, centrifuged gently, and incubated at 37° C. After 2 h, the system was added with 1U of DNase I and incubated at 37° C. for another 30 min to remove the DNA template. Then RNA was purified, usually by the magnetic bead purification method. The purified mRNA was dissolved in sterile enzyme-free water, and then quantitatively measured using Nanodrop One.

Liquid chromatography mass spectrometry (LC-MS) was used to determine the IVT capping rate of mRNA with different initial cap analogs. First, it is necessary to design a labeled DNA probe that matches the starting base of the transcript product mRNA, and usually the label is biotin. The streptavidin-labeled magnetic beads were washed, and incubated with the synthesized DNA probe, mRNA and 10×RNase H reaction buffer at room temperature for 30 min. The mixture was mixed slowly while incubating, then added with 20 μl of RNase. H (5U/μl), incubated at 37° C. for 3 h, and mixed thoroughly every half hour. After the incubation was completed, the magnetic beads were washed and added with 100 μL of 75% methanol heated to 80° C. The mixture was heated to 80° C. on a hot plate and kept for 3 min, and then placed on a magnetic stand. The supernatant was collected and dried at room temperature for 45 min to 10 μl using an evaporative centrifuge. The sample was then resuspended in 50 μl of 100 μM EDTA/1% MeOH, which was ready for LC-MS analysis to determine RNA capping during the transcription reaction. Since there was a significant difference in molecular weight between capped and uncapped bases, the capping rate of mRNA transcription initiated by different cap analogs can be determined through the difference in molecular mass. The specific results are shown in Tables 4-7.

TABLE 4

| | | | Increased | | Increased |
|---|---|---|---|---|---|
| | | | yield | | capping rate |
| | | | relative to | Cap- | relative to |
| | | | Comparative | ping | Comparative |
| | | Yield | Example 1 | rate | Example 1 |
| Number | Structural formula | (μg) | (%) | (%) | (%) |
| Example 1 | 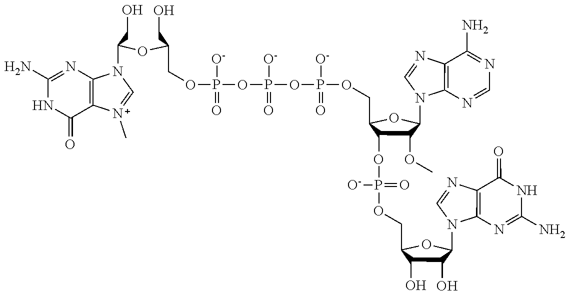 | 72 | 44 | 99.3 | 5.8 |
| Example 3 | 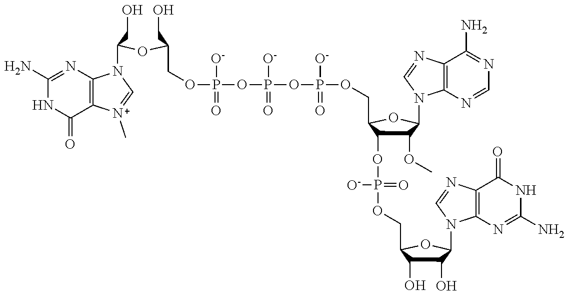 | 70 | 40 | 98.9 | 5.4 |
| Example 7 | 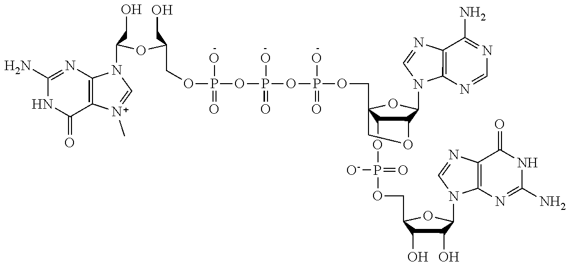 | 68 | 36 | 97.6 | 4.1 |
| Example 11 | 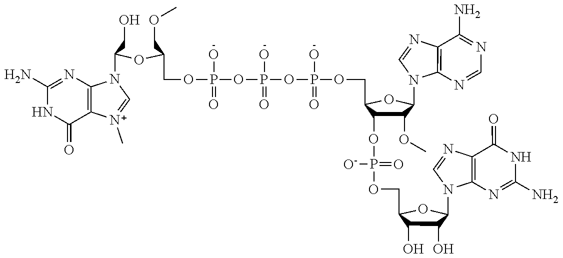 | 71 | 42 | 96.8 | 3.3 |
| Compara- tive Example 1 | 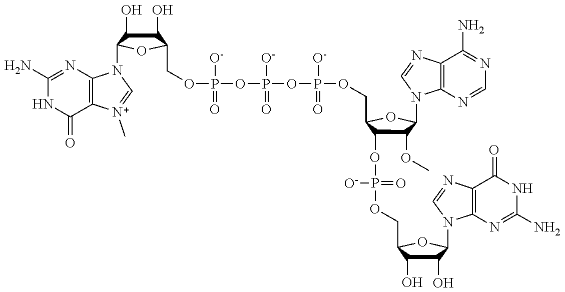 | 50 | — | 93.5 | — |

The data in Table 4 above show the results of in vitro transcription yield and capping efficiency of mRNA in Examples 1, 3, 7, 11 and Comparative Example 1 under the same experimental conditions. From Table 4, it is found that compared with the yield of 50 μg of Comparative Example 1, the yield of Example 1 increased by 44%, the yield of Example 3 increased by 40%, the yield of Example 7 increased by 36%, and the yield of Example 11 increased by 42%. Besides, compared with the capping rate of 93.5% of Comparative Example 1, the capping rate of Example 1 increased by 5.8%, the capping rate of Example 3 increased by 5.4%, the capping rate of Example 7 increased by 4.1%, and the capping rate of Example 11 increased by 3.3%. Compared with Comparative Example 1, it can be seen that the acyclic structure connected to R₁ and R₂ in the compounds of the present disclosure has a significant impact on the increase in yield and capping rate, which leads to significantly higher mRNA yield and capping rate than those of the Comparative Example 1 in which R₁ and R₂ are connected to a ring-closed structure.

TABLE 5

| | | | Increased capping |
|---|---|---|---|
| | | Capping | rate relative to |
| | | rate | Comparative |
| Number | Structural formula | (%) | Example 1 (%) |
| Example 1 | 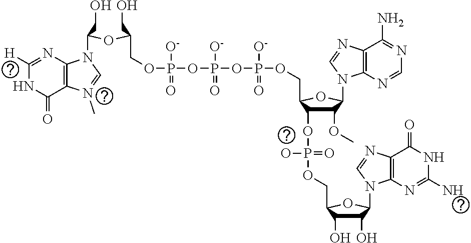 | 99.3 | 5.8 |
| Example 2 | 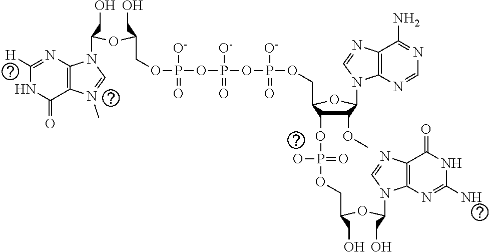 | 96.5 | 3 |
| Comparative Example 1 | 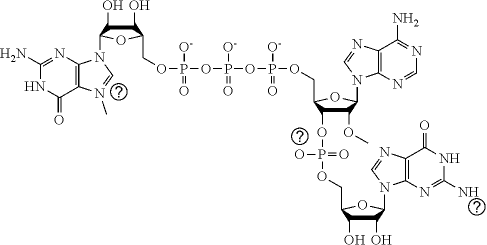 | 93.5 | — |

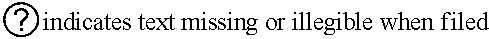

Furthermore, it can be seen from Table 5 that when R_{b} in the compounds of the present disclosure is a ring-closed structure (Example 1), it also has a certain impact on the improvement of the capping rate.

In addition, the summary results of the in vitro transcription yield and capping efficiency of mRNA in Examples 1-4, 6, 7, 11 and Comparative Examples 1-2 under the same experimental conditions are shown in Tables 6-7.

TABLE 6

| Number | Yield (μg) |
|---|---|
| Example 1 | 72 |
| Example 2 | 58 |
| Example 3 | 70 |
| Example 4 | 45 |
| Example 6 | 44 |
| Example 7 | 68 |
| Example 11 | 71 |
| Comparative Example 1 | 50 |
| Comparative Example 2 | 41 |

TABLE 7

| Number | Capping rate (%) |
|---|---|
| Example 1 | 99.3 |
| Example 2 | 96.5 |
| Example 3 | 98.9 |
| Example 4 | 93.3 |
| Example 6 | 95.2 |
| Example 7 | 97.6 |
| Example 11 | 96.8 |
| Comparative Example 1 | 93.5 |
| Comparative Example 2 | 90.1 |

Therefore, it can be seen from the experimental results that the oligonucleotide primers with an acyclic nucleoside structure for initial capping of the present application have higher in vitro transcription yield and capping efficiency of mRNA. These results show that the cap structures provided by the present disclosure have better substrate adaptability and higher reaction efficiency under the catalysis of enzyme, so that they can produce higher in vitro transcription yield and capping efficiency of RNA.

Test Example 2: Determination of the binding ability of mRNA to RIG I

RIG-I mainly includes two repeated caspase activation and recruitment domains (CARD) at the N-terminus, a helicase structure in the middle and a RNA domain at the C-terminus. The overexpression of the N-terminal CARD domain of RIG-I can promote cells to secrete type I interferon (IFN) even in the absence of viral infection. Therefore, this domain is mainly responsible for transmitting signals downstream. RIG-I can recognize exogenous RNA, mediate the production of cytokines and chemokines, and recruit innate immune cells to trigger immune responses. mRNA with high immunogenicity is easily recognized and combined by RIG. Thus, mRNA with strong binding ability to RIG-1 has high immunogenicity.

In this study, 293T cells were transfected with the eGFP mRNAs prepared by in vitro transcription initiated by the oligonucleotide primers with an acyclic nucleoside structure for initial capping in Examples 1-4, 6, 7, and 11 and the cap analogs in Comparative Examples 1-2, and collected 24 hours later. The intracellular protein RIG I and RNA bound thereto were co-immunoprecipitated by the RNA co-immunoprecipitation method. Finally, reverse transcription and real-time quantitative PCR were performed on these mRNAs.

The specific cell culture conditions were the same as above. Cells were collected 24 hours after transfection. First, a fixing solution was added for incubation. After 10 min, glycine solution of an appropriate concentration was added to terminate the reaction, and cells were collected. The collected cells were lysed using the lysis solution, centrifuged at 12,000 rpm and 4° C. for 10 min. The supernatant was collected and incubated with an appropriate amount of RIG-I or IgG antibody on a shaker at 4° C. overnight. Then 20 μl of Protein A/G magnetic beads were added, incubated at 4° C. for 4 h, and washed on a magnetic stand. After washing was completed, RNA was extracted and used for subsequent RT-qPCR to determine the expression results. The relative binding ability results of different cap analog nucleotide mRNAs to RIG-1 are shown in the following Table 8:

TABLE 8

| Different cap analogs | Relative binding ability to RIG-I |
|---|---|
| Example 1 | 1 ± 0.12 |
| Example 2 | 2.4 ± 0.34 |
| Example 3 | 1.1 ± 0.21 |
| Example 4 | 2.8 ± 0.62 |
| Example 6 | 1.4 ± 0.32 |
| Example 7 | 2.1 ± 0.21 |
| Example 11 | 1.5 ± 0.25 |
| Comparative Example 1 | 3.1 ± 0.51 |
| Comparative Example 2 | 4.0 ± 0.31 |

It can be seen from the experimental data in Table 8 that compared with the relative binding ability to RIG-I of 3.1±0.51 in Comparative Example 1, Example 1 was 1±0.12, Example 3 was 1.1±0.21, Example 6 was 1.4±0.32, and Example 11 was 1.5±0.25, showing that the relative binding ability to RIG-I was significantly reduced, indicating that mRNAs synthesized with the capping analogs containing an acyclic nucleoside structure of the present disclosure had a significantly lower cell immunogenicity than that of Cleancap.

Test example 3: Cell protein expression test

The eGFP coding sequence was used as a DNA template, and the cap analogs of Examples 1-4 and Comparative Examples 1-2 were used to initiate in vitro transcription. The different mRNA products obtained were then transfected into 293T cells.

293T cells were plated at (0.5-1)×10⁵ cells (24-well plate). It is recommended to use cells within 50 generations for transfection experiments. The cells were required to be passaged again 24 h before transfection. The addition of antibiotics to the culture medium had no effect on the transfection effect. During transfection, the cell density was generally 60-80%. Each well was transfected with 2 μg of mRNA. Lipofectamine MessengerMAX Transfection Reagent (Invitrogen) was used as the transfection reagent and operation was carried out according to its usage instructions. The transfected cells were placed in a 37° C., CO₂ incubator and replaced with fresh complete culture medium 4-6 h after transfection. After incubation for 24 h in a 37° C., CO₂ incubator, the fluorescence intensity of GFP was observed under a fluorescence microscope. The results are shown in FIGS. 7 and 8. From the results, it can be clearly seen that the expression efficiency of the mRNA of the present disclosure was significantly higher than that of the comparative examples, and neither of them caused obvious cell death. This result shows that the oligonucleotide primers with an acyclic nucleoside structure for initial capping of the present application have higher expression efficiency, that is, when the oligonucleotide primer with an acyclic nucleoside structure for initial capping of the present disclosure is used in mRNA synthesis, the effective protein translation efficiency is significantly higher than that of the Cleancap (Comparative Example 1) cap structure.

Although the embodiments of the present disclosure have been shown and described, those of ordinary skill in the art will understand that various changes, modifications, substitutions and variations may be made to these embodiments without departing from the principles and spirit of the present disclosure. The scope of the present disclosure is defined by the appended claims and their equivalents.

1. An oligonucleotide primer with an acyclic nucleoside structure for initial capping, having a structure of 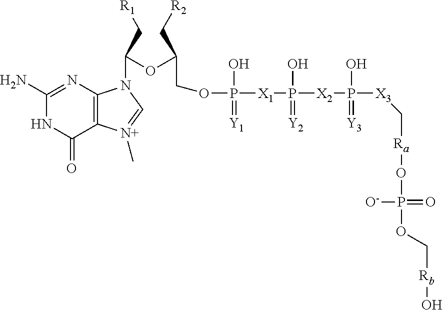
wherein, R₁ and R₂ are independently selected from the group consisting of H, OH, alkyl, O-alkyl, and halogen;
X₁, X₂ and X₃ are independently selected from the group consisting of O, CH₂ and NH;
Y₁, Y₂ and Y₃ are independently selected from the group consisting of O, S, Se and BH₃;
Rₐ and R_{b} are independently 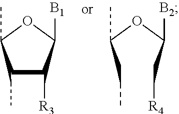
R₃ and R₄ are independently selected from the group consisting of hydrogen, hydroxyl, halogen, substituted or unsubstituted O-alkyl, substituted or unsubstituted S-alkyl, substituted or unsubstituted NH-alkyl, substituted or unsubstituted N-dialkyl, substituted or unsubstituted O-aryl, substituted or unsubstituted S-aryl, substituted or unsubstituted NH-aryl, substituted or unsubstituted O-aralkyl, substituted or unsubstituted S-aralkyl, and substituted or unsubstituted NH-aralkyl; and
B₁ and B₂ are independently natural, modified, or non-natural nucleobases.

2. The oligonucleotide primer according to claim 1, wherein X₁, X₂ and X₃ are independently O; and
Y₁, Y₂ and Y₃ are independently O.

3. The oligonucleotide primer according to claim 1, wherein R_{b} is

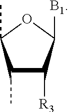

4. The oligonucleotide primer according to claim 1, wherein Rₐ and R_{b} are independently

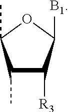

5. The oligonucleotide primer according to claim 1, wherein R₁ and R₂ are independently selected from the group consisting of H, OH, halogen, alkyl, and substituted or unsubstituted O-alkyl.

6. The oligonucleotide primer according to claim 1, wherein R₃ and R₄ are independently selected from the group consisting of hydrogen, hydroxyl, halogen, and substituted or unsubstituted O-alkyl.

7. The oligonucleotide primer according to claim 1, wherein R₁ and R₂ are independently selected from the group consisting of H, OH, F, methyl, ethyl, methoxy, ethoxy, —O-methylene-O-methyl, —O-ethylene-O-methyl, —O-methylene-O-ethyl, and —O-ethylene-O-ethyl.

8. The oligonucleotide primer according to claim 1, wherein R₃ and R₄ are independently selected from the group consisting of H, OH, F, methoxy, ethoxy, —O-methylene-O-methyl, —O-ethylene-O-methyl, —O-methylene-O-ethyl, and —O-ethylene-O-ethyl.

9. The oligonucleotide primer according to claim 1, having a structure selected from the group consisting of:

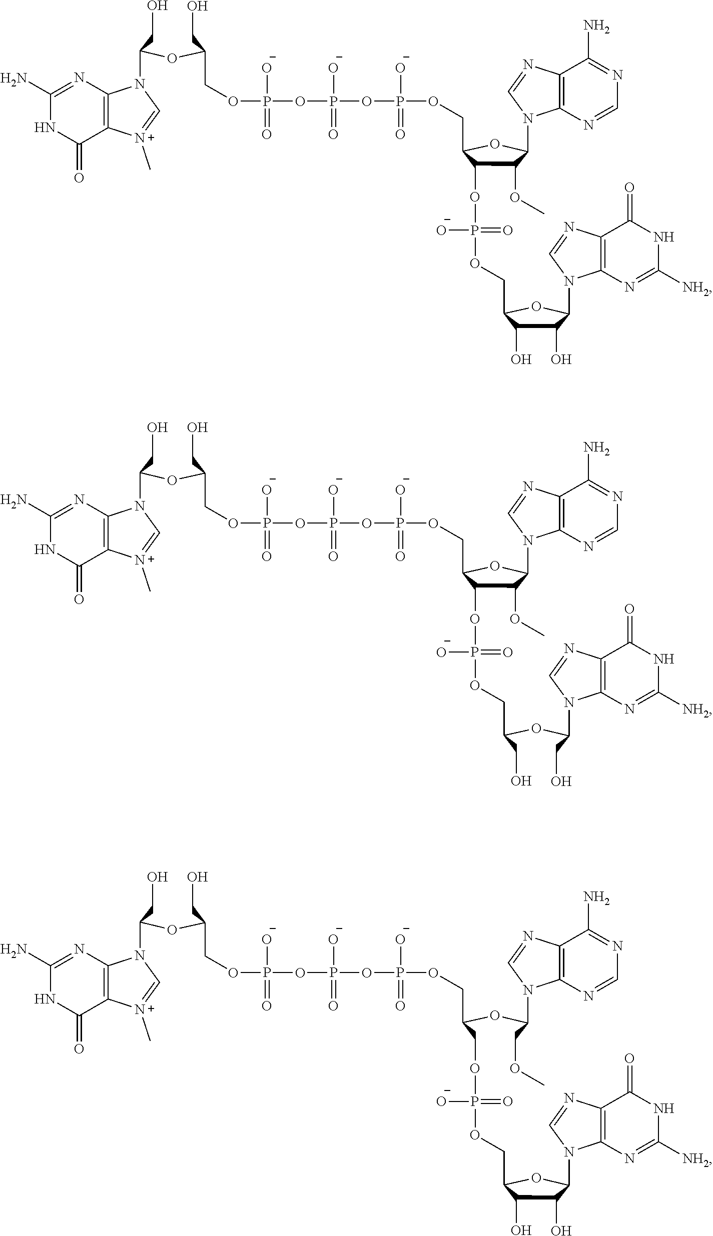

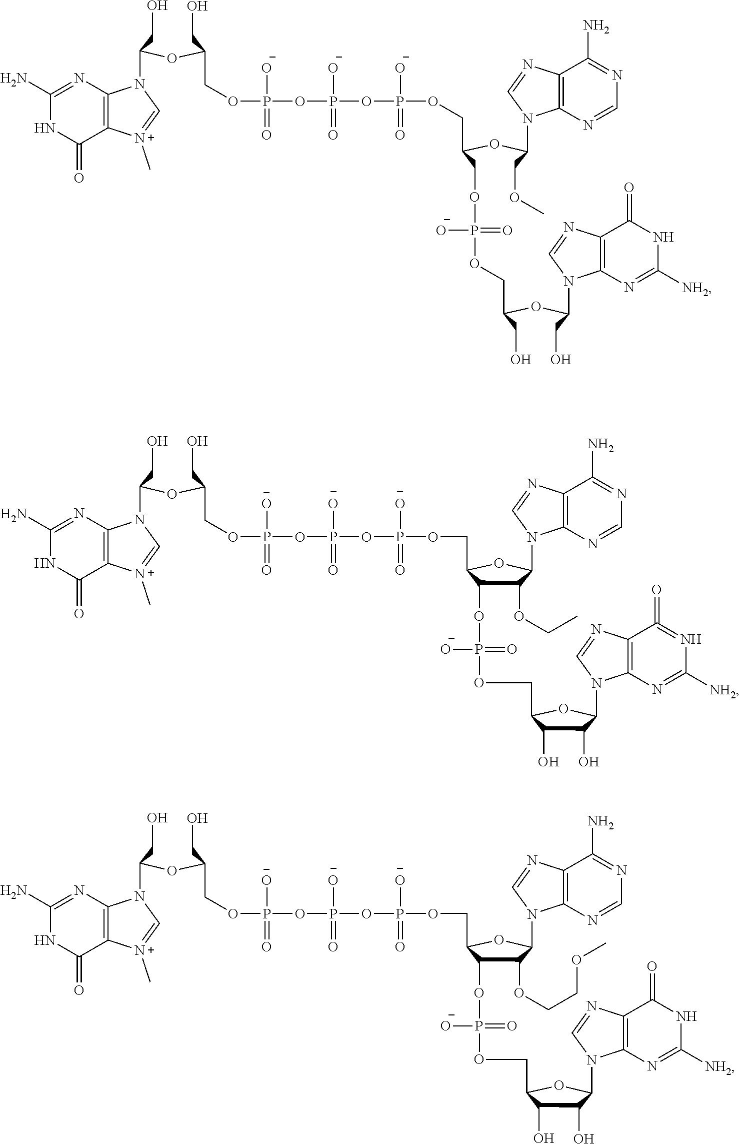

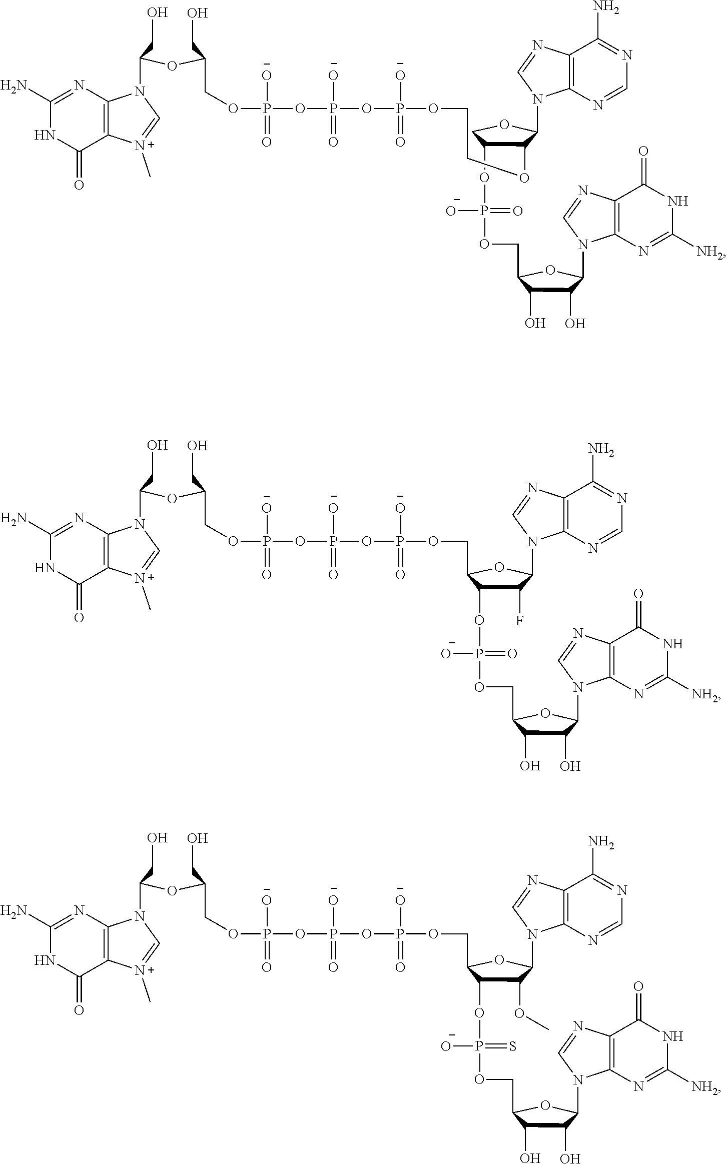

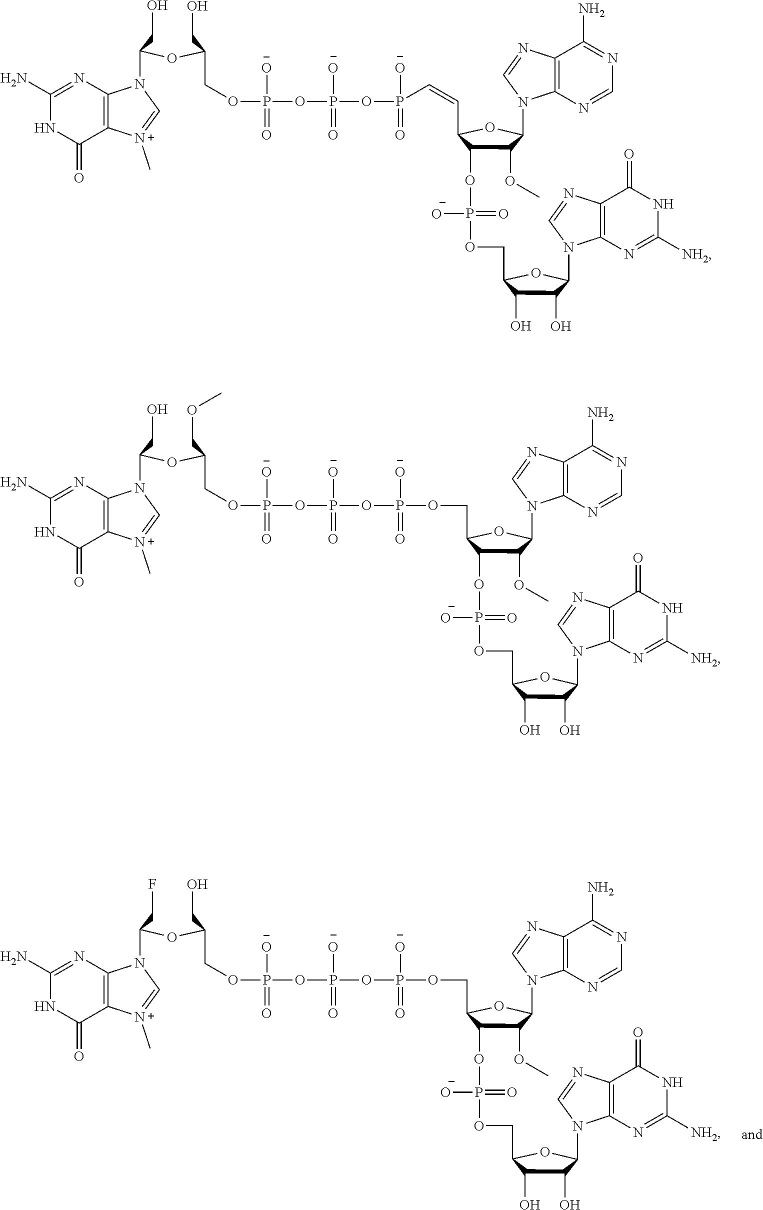

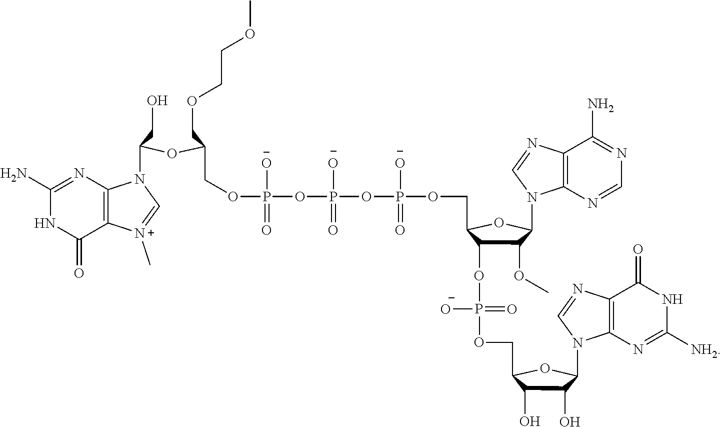

10. The oligonucleotide primer according to claim 1, having a structure selected from the group consisting of:

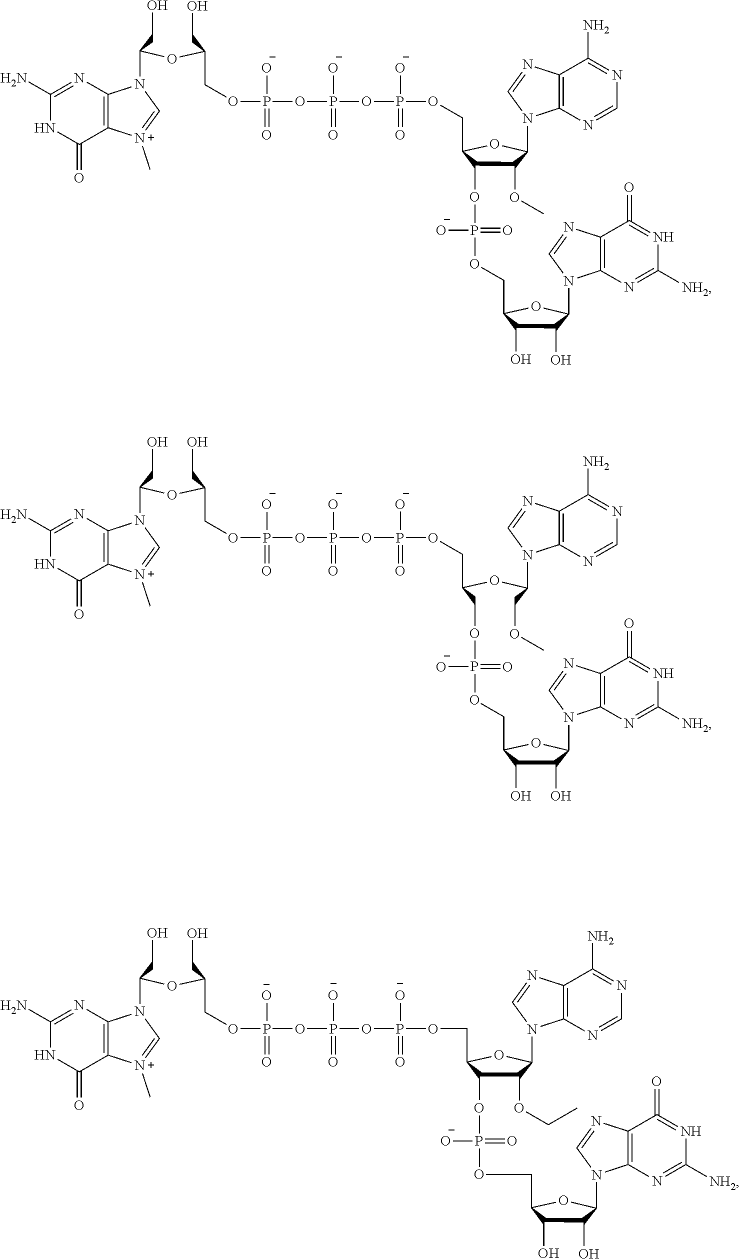

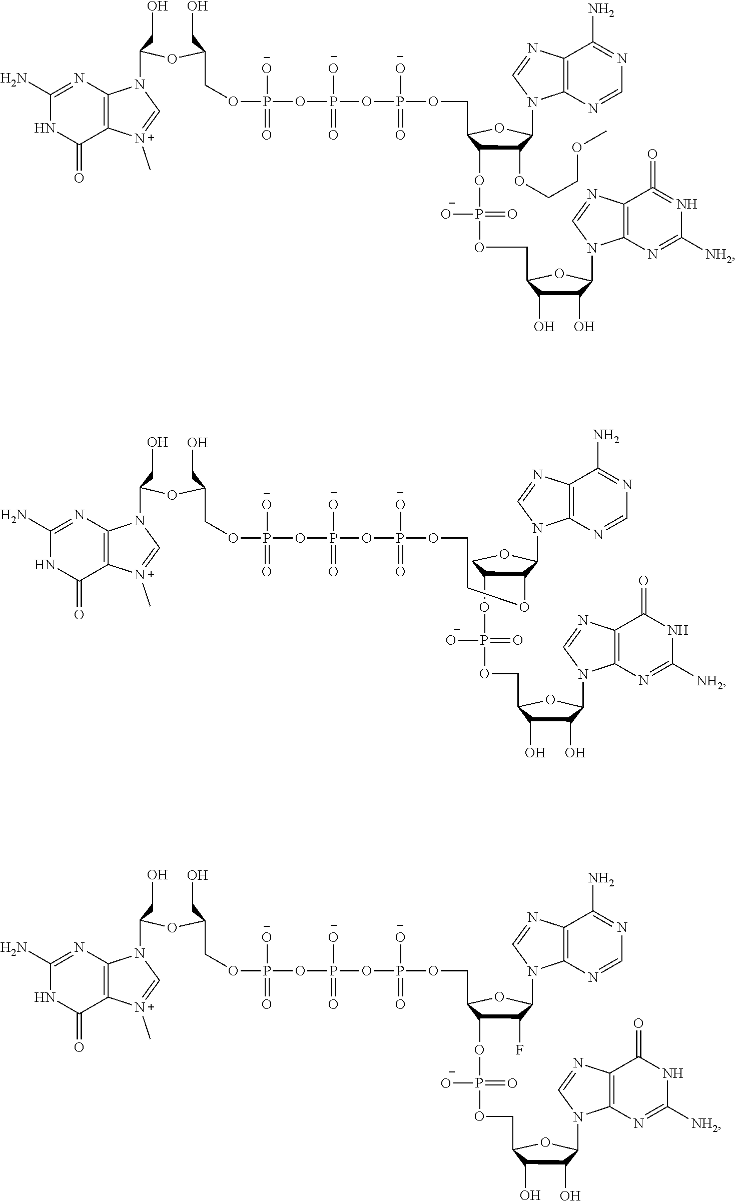

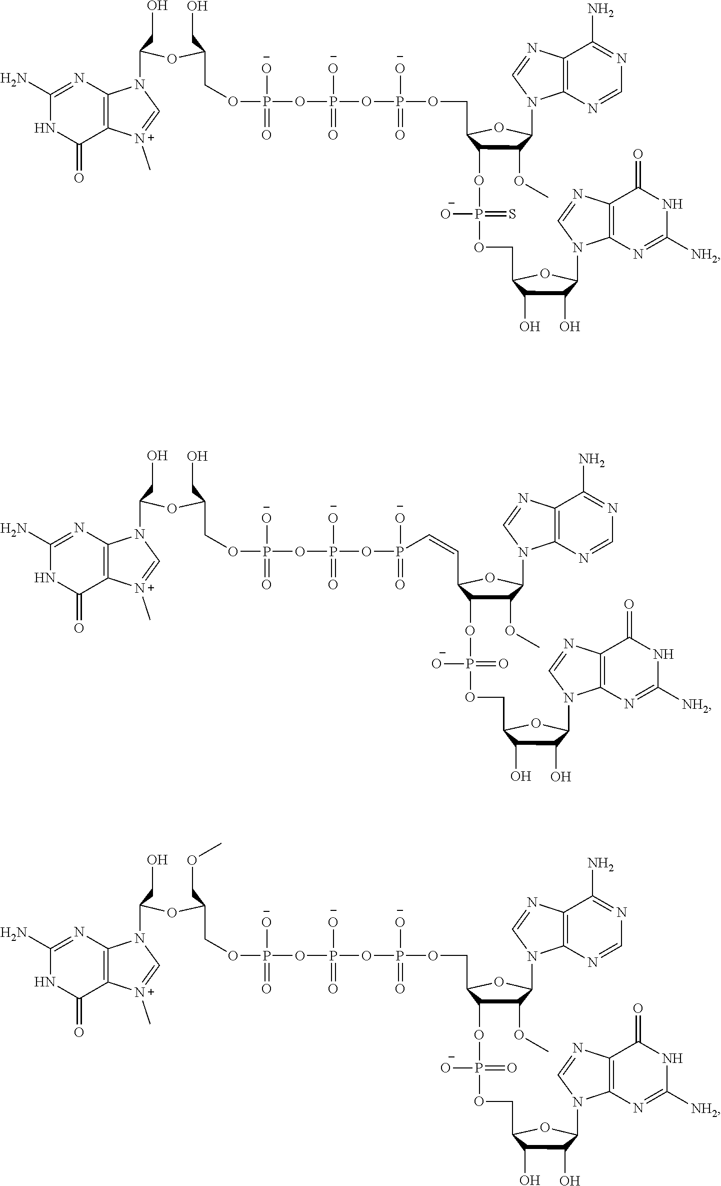

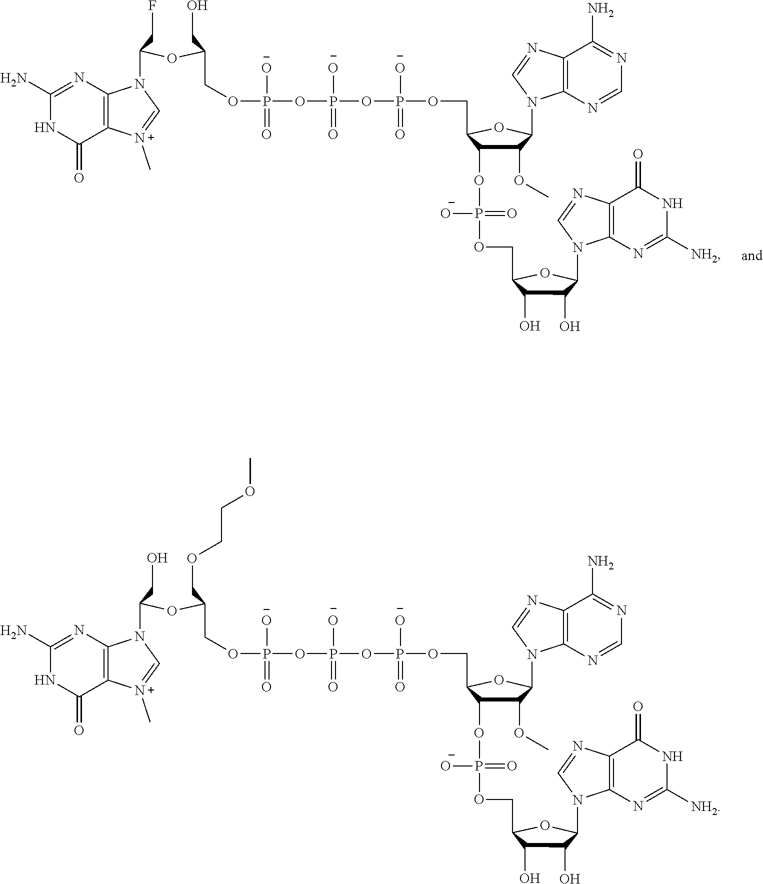

11. The oligonucleotide primer according to claim 1, having a structure selected from the group consisting of:

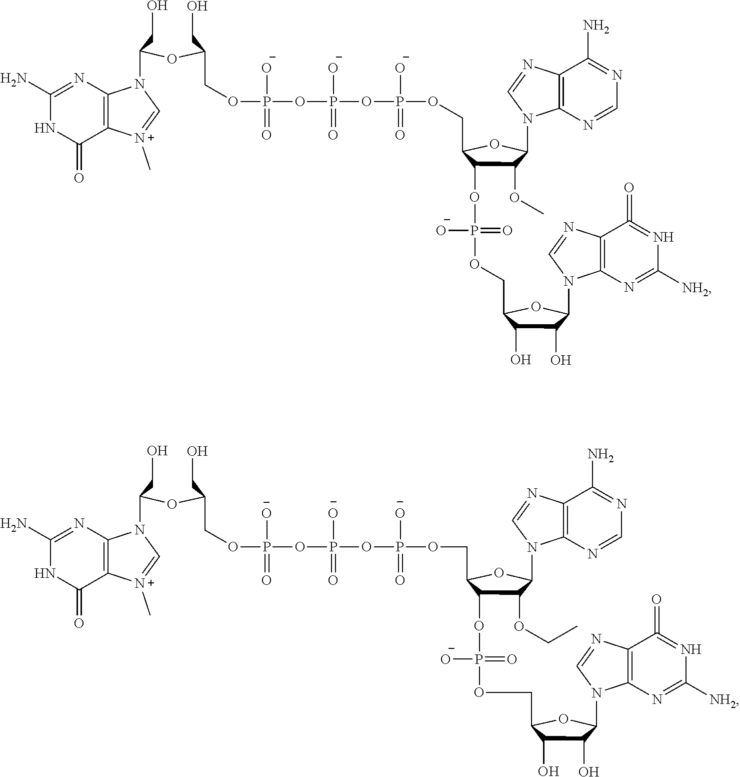

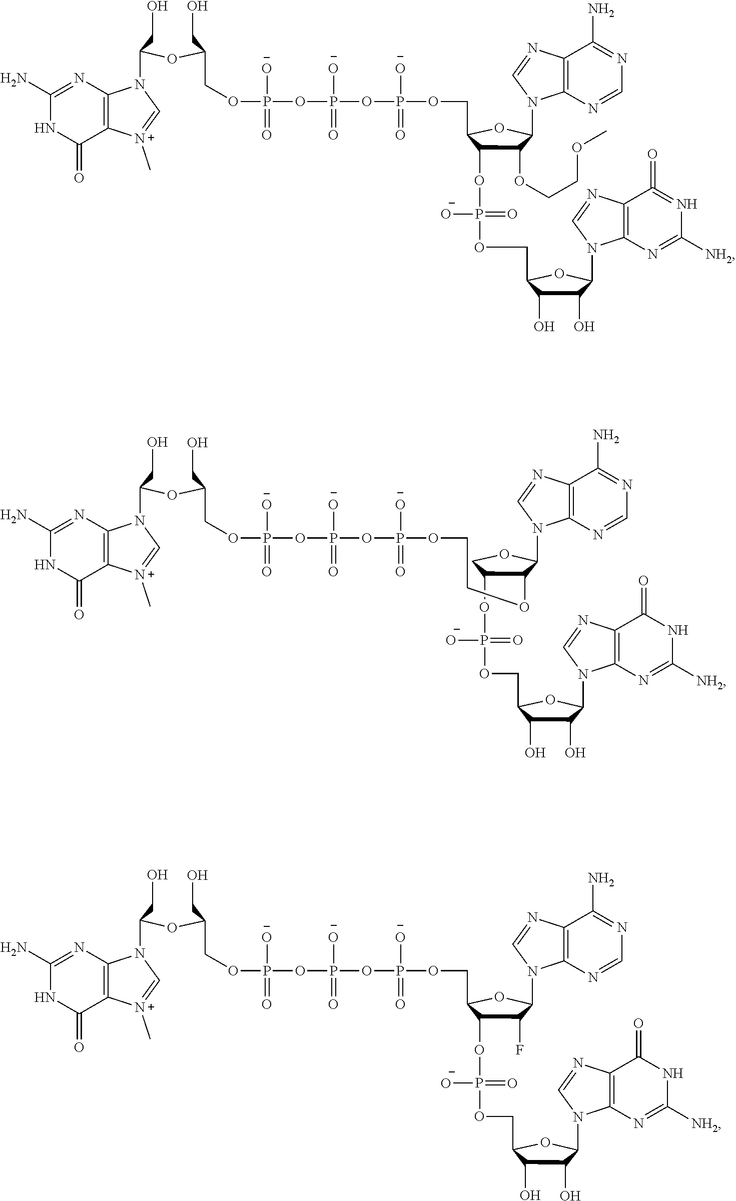

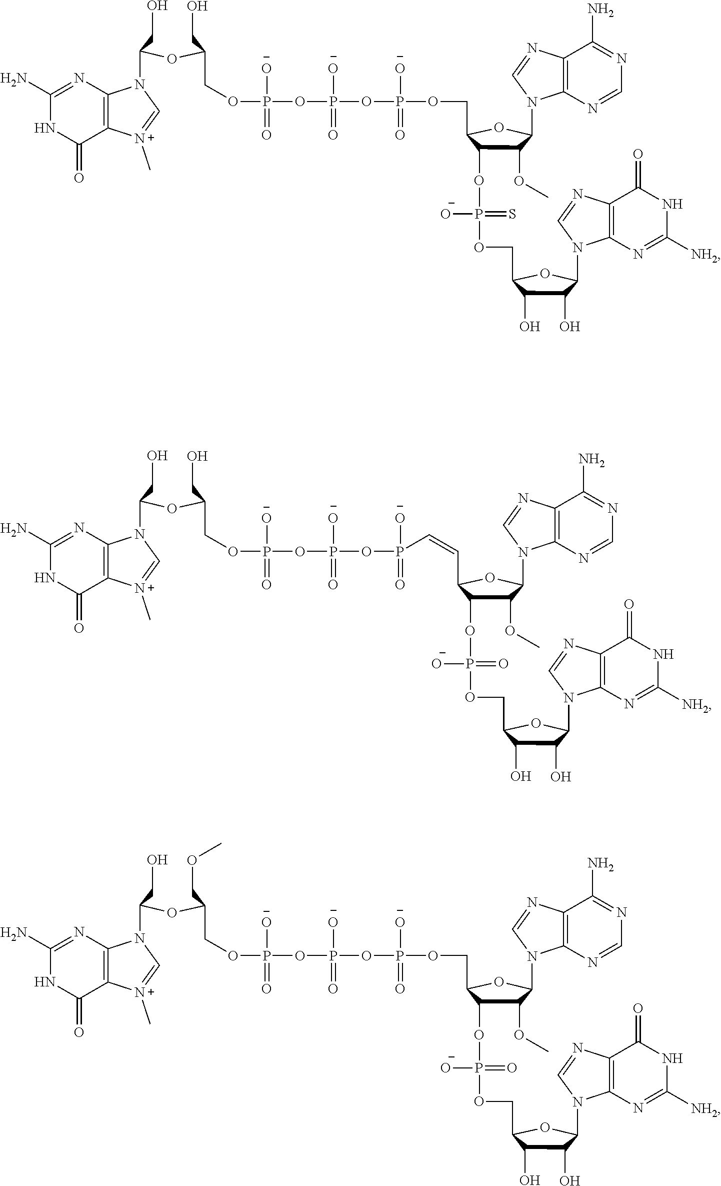

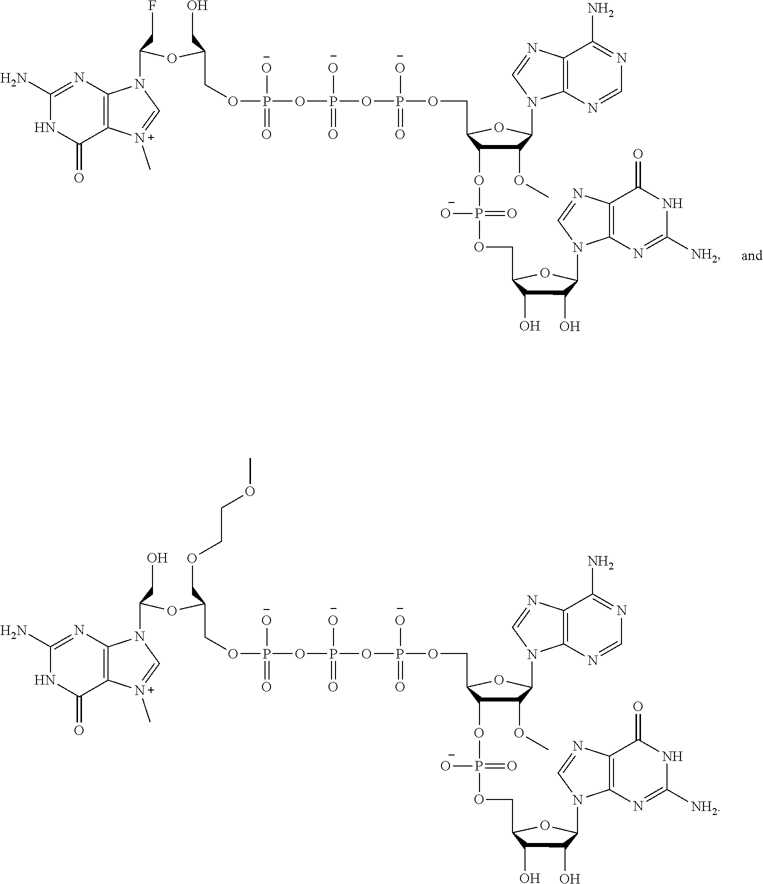

12. The oligonucleotide primer according to claim 1, having a structure selected from the group consisting of:

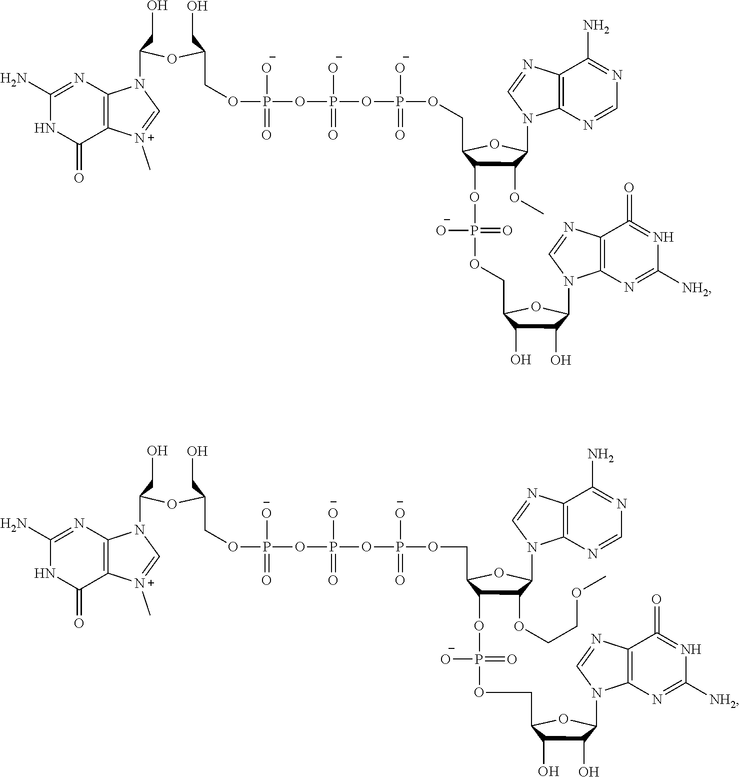

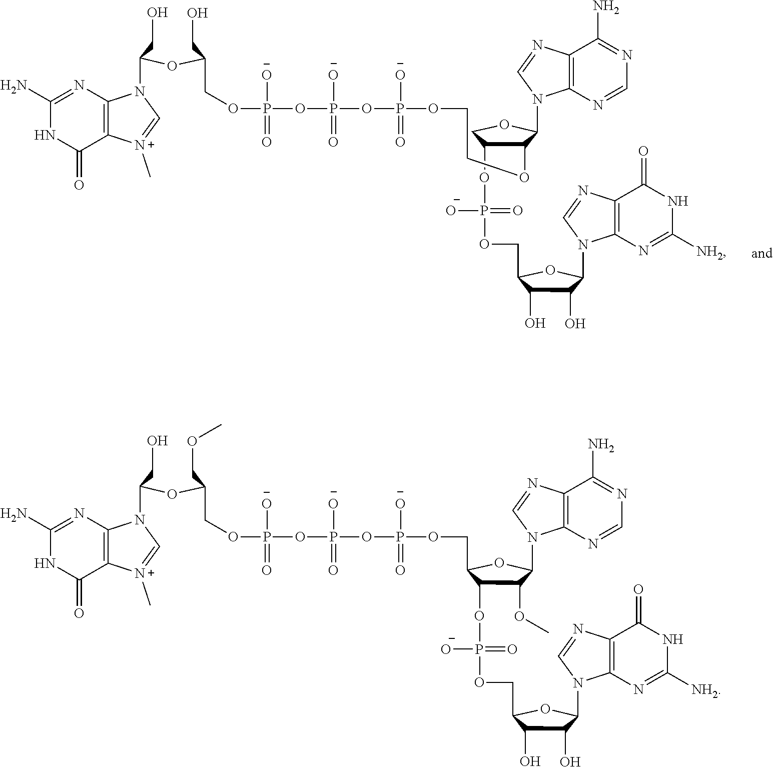

13. The oligonucleotide primer according to claim 1, having a structure of:

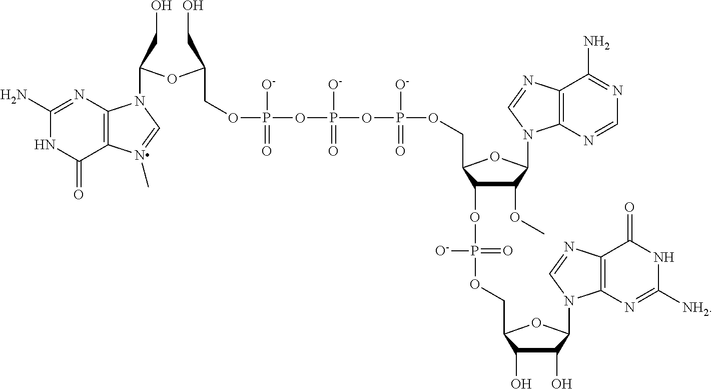

14. A method for preparing the oligonucleotide primer according to claim 1, comprising steps of: (1) synthesizing m7UrGDP-Im: synthesizing sugar ring-opening acyclic nucleoside from guanosine, and subjecting the acyclic nucleoside to diphosphorylation, N7 methylation, and polyphosphate imidazolation sequentially to synthesize m7UrGDP-Im; (2) preparing a phosphate bond-linked dinucleotide: coupling acyclic or non-acyclic phosphoramidite monomer with acyclic or non-acyclic disubstituted nucleoside monomer under the action of tetrazole to form a first phosphate bond, removing a protecting group by acid, then introducing a second phosphate, and finally performing hydrolyzation to obtain a phosphate bond-linked dinucleotide; and (3) synthesizing an oligonucleotide primer with an acyclic nucleoside structure for initial capping: reacting m7UrGDP-Im with the phosphate bond-linked dinucleotide to obtain the oligonucleotide primer with an acyclic nucleoside structure for initial capping;
wherein, the phosphoramidite monomer has a structure of 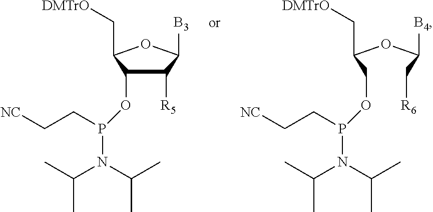
wherein, R₅ and R₆ are independently selected from the group consisting of H, OH, alkyl, O-alkyl, and halogen; and B₃ and B₄ are independently natural, modified, or non-natural nucleobases.

15. A method for in vitro co-transcriptional capping for RNA, comprising steps of: (1) providing the oligonucleotide primer according to claim 1;
(2) providing a DNA template; and
(3) performing in vitro transcription of mRNA.

16. A kit comprising the oligonucleotide primer according to claim 1.

17. A complex comprising the oligonucleotide primer according to claim 1 and a DNA template, wherein the DNA template comprises a promoter region containing a transcription start site, the transcription start site has a first nucleotide at nucleotide position +1 and a second nucleotide at nucleotide position +2, the base in Rₐ of the oligonucleotide primer is complementary to the nucleobase at position +1 of the DNA template, and the base in R_{b} of the oligonucleotide primer is complementary to the nucleobase at position +2 of the DNA template.

18. An mRNA molecule comprising the oligonucleotide primer according to claim 1.
